(19) United States
(12) Patent Application Publication
(10) Pub. No.: US 2019/0151604 A1
(43) Pub. Date: May 23, 2019

(54) DEVICE, SYSTEM AND METHOD FOR MECHANICAL CUTANEOUS NERVE STIMULATION FOR PAIN, STROKE, MOOD, BREATHING, MOVEMENT, SLEEP, AND VASCULAR ACTION

(71) Applicant: THE REGENTS OF THE UNIVERSITY OF CALIFORNIA, Oakland, CA (US)

(72) Inventors: Ronald M. HARPER, Los Angeles, CA (US); Eberhardt K. SAUERLAND, Reno, NV (US)

(21) Appl. No.: 16/300,211

(22) PCT Filed: May 11, 2017
(86) PCT No.: PCT/US17/32214

§ 371 Date: Nov. 9, 2018

Related U.S. Application Data
(60) Provisional application No. 62/334,799, filed on May 11, 2016.

(51) Int. Cl. A61M 21/02; A61H 39/00

(52) U.S. Cl. CPC A61M 21/02; A61H 39/007; A61H 2201/0107; A61H 2201/5007; A61N 2007/0026; A61H 2205/04; A61H 2205/022; A61H 2201/5097; A61H 2205/062

(57) ABSTRACT

The invention describes a non-electrically stimulation system, devices, and methods for non-invasive vibration stimulation procedures for treating, reducing and alleviating symptoms associated with pain, e.g., headache pain, migraine pain, and joint pain, stroke, mood, vascular action, breathing disorders during sleep and waking, limb, head and swallowing movement disorders, and sleep induction. The invention also describes a system, devices, and methods for non-invasive vibration stimulation procedures for retraining breathing.

### CROSS-REFERENCE TO RELATED APPLICATIONS

This application is a national stage entry of PCT Application No. PCT/US17/32214, filed May 11, 2017, which claims priority to U.S. provisional application No. 62/334,799 filed on May 11, 2016, both of which are incorporated herein by reference in their entirety.

### BACKGROUND OF THE INVENTION

The need for simple, non-invasive interventions for pain, sleep induction, and breathing and movement disorders is substantial. Chronic regional pain is common, and migraine pain has a one-year prevalence of approximately 12% of the United States population. The pain is often debilitating, with annual costs from migraine alone in excess of 36 billion dollars in the United States (Huet al., 1999). Some forms of migraine show unique characteristics and have different nomenclature. One form is trigeminal neuralgia, characterized by sudden catastrophic pain, triggered by mild stimulation, typically to the upper lip. Another is the so-called “burning mouth” syndrome, characterized by long-lasting, severe pain in the tongue or oral cavity, i.e., stomatodynia and glossodynia. Other forms appear to result from hormonal or other processes which constrict or alter diameter of the cerebral vasculature. The variation in symptoms emphasizes the complexity of migraine and associated pain syndromes, and the difficulty in suitable interventions. Cranial nerve 5, the trigeminal nerve, mediates many of the sources of pain (Akerman et al., 2013). As with migraine pain, trigeminal neuropathy pain is often accompanied by ancillary autonomic or other issues; diminished salivation is one such frequent concern, and reduced fluid secretion with sinus pain is another; both issues are addressed with the efforts here.

Migraine and pain from trigeminal origins comprise a large proportion of pain sources, but cervical, shoulder, lower body, and limb pain are also major sources of concern. Pain originating from the knees, ankles or other portions of the limb is common.

Classical approaches to pain intervention include a range of pharmacologic agents (Olesen and Ashina, 2011), including serotonin agonists and nitric oxide antagonists, electrical stimulation of the forehead, deep brain structures, or nerves within the brain or deep to the surface (Meng et al., 2013; Mosqueira et al., 2013, Schoenen et al., 2013), including transcranial electrical stimulation (DaSilva et al., 2012), and Botox application (Silberstein et al., 2000) to reduce innervation from tension in scalp muscles. Those interventions vary in effectiveness. Pharmaceutical agents often have severe side effects, with even aspirin exerting significant bleeding risks, electrical stimulation poses other risks for cutaneous injury, and muscle paralysis (Botox) procedures are often transient. Interventions for trigeminal neuralgia include anti-epileptic or other medications, vascular decompression surgery (Janetta, 1980), or lesioning of areas of the 5th cranial nerve; none of these options are ideal. Medications often exert serious cognitive, cardiovascular, or mood side effects, or are not tolerated by subjects, lesions often result in other sensory loss, such as touch or temperature, in addition to effects on pain, and the pain is often untouched. Surgery for such lesions is invasive, with associated infection risks. The stimulation procedures requiring deep brain stimulation or access to nerves involve major surgery with substantial anesthetic and infection risks and uncertain outcomes (Pedersen et al., 2013).

Accordingly, there is a need in the art for alternative, non-invasive, devices and procedures that are easy to use for treating, reducing and alleviating symptoms associated with pain, e.g., headache and migraine pain, pain from trigeminal neuropathy, regional pain of the neck, shoulders and other body parts, and a need to intervene for disordered breathing during sleep, mood, cardiovascular and movement disorders, and neural injury accompanying stroke. The present invention satisfies those needs.

### SUMMARY OF THE INVENTION

In one aspect, the invention relates to a system for stimulating one or more sensory fibers of a nerve, comprising: a device comprising a vibration source and a first magnet in contact with the vibration source, such that vibrational energy is capable of transferring to the first magnet; and a second magnet; wherein the first magnet and the second magnet are releasably engageable via a magnetic field. In one embodiment, the second magnet is further attached to an adhesive patch. In one embodiment, the vibration source is a vibration motor. In one embodiment, the vibration source is connected to and powered by a control unit. In another embodiment, the control unit is controlled by a computing device. In another embodiment, the computing device controls the control unit wirelessly.

In another aspect, the invention relates to a system for stimulating one or more sensory fibers of a nerve, comprising: a device comprising a vibration source and a first mechanical connector in contact with the vibration source, such that vibrational energy is capable of transferring to the mechanical connector; and a second mechanical connector attached to an adhesive patch; wherein the first mechanical connector and the second mechanical connector are releasably engageable via friction or other type of mechanical retention. In one embodiment, the vibration source is a vibration motor. In one embodiment, the first mechanical connector is a snap connector. In another embodiment, the snap connector is a female snap connector. In one embodiment, the vibration source is connected to and powered by a control unit. In another embodiment, the control unit is controlled by a computing device. In another embodiment, the computing device controls the control unit wirelessly.

In one aspect, the invention relates to a method of stimulating one or more sensory fibers of a nerve in a subject, wherein the nerve is selected from the group consisting of ophthalmic nerve V1, maxillary nerve V2, mandibular nerve V3, cranial nerve 5, cranial nerve 7, cranial nerve 9, cranial nerve 10, spinal nerve C1, spinal nerve C2, spinal nerve C3, and spinal nerve C4, as well as spinal nerves from C5 through sacral 5 nerves. The method comprising placing a vibration source device on or proximal to the skin of the subject. In one embodiment, the vibration source device further comprises a first magnet, the method comprising placing the first magnet of the device on a portion of the skin of the subject and providing a second magnet, wherein the first magnet and the second magnet are releasably attracted to each other via a magnetic field through at least a portion of the skin of the subject. In another embodiment, the vibration source device further comprises a first magnet, the method comprising providing an adhesive patch fitted with a second magnet, and placing the adhesive patch on to the skin of the subject, wherein the two magnets are releasably attached to each other via a magnetic field. In another embodiment, the vibration source device further comprises a first mechanical connector, the method comprising providing an adhesive patch fitted with a second mechanical connector, placing the adhesive patch on to the skin of the subject, wherein the two mechanical connectors are releasably attached to each other by friction or other type of mechanical retention. In another embodiment, the vibration source device comprises a first vibration device and magnet which is releasably magnetically coupled to a silicon implant in one nasal cavity through a second magnet on the implant.

In one embodiment, stimulation leads to treating, alleviating, or preventing a condition in a subject. In one embodiment, the condition is selected from the group consisting of pain of the head, pain of the oral cavity, pain of the neck, pain of the shoulder, pain in one or several of the nasal or frontal sinuses, pain of the face, pain of the dura covering the brain, and pain in localized areas of other parts of the limbs or body. In another embodiment, the condition is a cardiovascular condition. In another embodiment, the condition is a cardiovascular condition selected form the group consisting of atrial fibrillation, sinus tachycardia, ventricular tachycardia, long Q-T syndrome, hypertension, hypotension, or extremely variable blood pressure. In another embodiment, the condition is selected from the group consisting of ischemic stroke, a mood disorder, depression, anxiety, post-traumatic stress disorder, epilepsy, movement disorder, including falling, gait disorders, tremor, coughing, swallowing, hiccup, or involuntary tic. In another embodiment, the condition is selected from the group consisting of obstructive pulmonary disease (COPD), asthma, obstructive sleep apnea, central apnea, Cheyne-Stokes breathing, periodic breathing, and hypoventilation. In one embodiment, the stimulation leads to retraining breathing in a subject. In another embodiment, the stimulation leads to inducing sleep. In one embodiment, the stimulation leads to induction of sleep in a subject.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A and 1B show an anatomic chart depicting the corresponding input from skin mechanoreceptors and pain receptors in the head and neck area to the brainstem.

FIG. 2 is an anatomic chart depicting the auricular branch of the vagus, i.e., cranial nerve 10.

FIGS. 3A and 3B show an anatomic chart depicting the corresponding input from auricle mechanoreceptors and pain receptors to the brainstem.

FIGS. 4A and 4B show an anatomic chart depicting two views of the distribution of the auricular branch of the vagus, i.e., cranial nerve 10 over the external ear. FIG. 4A depicts the lateral surface of the external ear and within the corresponding scheme, the auricular branch of vagus nerve (ABVN), the great auricular nerve (GAN), the auriculotemporal nerve (ATN), and the superficial temporal artery (STA). FIG. 4B depicts the medial surface of the external ear and within the corresponding scheme, the auricular branch of vagus nerve (ABVN), the lesser occipital nerve (LON), and various vessels (V).

FIG. 5 depicts a schematic of an exemplary embodiment 100 of a device of the invention while appended to a tissue area of auricle.

FIG. 6 includes a photograph depicting an exemplary embodiment of a device of the invention, including a motor, with attached magnet and power supply cable.

FIG. 7 is a photograph depicting an exemplary embodiment of a stimulation power supply for a coin motor. The controller is designed to control the amplitude, timing, pulse train length and interpulse interval of pulses for the vibration leads. The controller has an On-Off switch (1), two output ports for vibrator leads (2), and two push buttons for a 3-step up-down fine adjustment of amplitude to the coin motors (3, amplitude up, and 4, amplitude down). Pulse characteristics are remotely programmed with an Android device, and transmitted to the controller via Bluetooth signals, where those parameters are stored within the memory of the controller, and optionally, stored on the tablet as well.

FIGS. 8A and 8B include two photographs depicting two modes of appending a device of the invention to the auricle. In FIG. 8A the vibrating coin motor is placed on the lateral surface of the auricle helix tail with a holding magnet on the medial side. In FIG. 8B the placement of the coin motor and holding magnet is reversed, with the motor on the medial surface of auricle and the circular holding magnet on the lateral (external) surface.

FIGS. 9A and 9B include two photographs depicting a mode of appending a device of the invention to the auricle, where the position of the vibrating coin motor is optimized to primarily affect select cranial nerve sensory fibers over others. The vibrating coin motor is placed in the posterior concha (FIG. 9A), and is held in place on the medial side of the auricle with the holding magnet (FIG. 9B).

FIGS. 10A and 10B include two photographs depicting a mode of appending a device of the invention to the auricle in primarily vagal (cranial nerve X) territory, by reversing the order of the vibrating motor and holding magnet in more difficult access circumstances. The coin motor is placed on the auricle medial to the posterior border of the concha (FIG. 10A), with the holding magnet on the lateral (external) surface (FIG. 10B).

FIG. 11 depicts a schematic of an exemplary embodiment 200 of a device of the invention while appended to a tissue area of auricle, including organization of delivery system for sites on external ear. Compared to the exemplary embodiment 100, a handle (such as the plastic head of a thumb tack) is attached to the holding magnet to assist placement. However, the holding magnet typically will be attracted to the magnetic field of the magnet on the opposite side of the tissue, and find its place automatically.

FIG. 12 includes a photograph depicting a mode of fabricating a handle for the free-standing magnet.

FIGS. 13A and 13B include two photographs depicting a mode of appending a device of the invention to the auricle, where the vibrating coin motor is in the back of (medial to) the auricle (FIG. 13A), specifically located on the eminentia conchae for maximal stimulation of the auricular branch of the vagus (CN 10), while the holding magnet is placed with the handle in front of (lateral to) the auricle (FIG. 13 B).

FIGS. 14A and 14B include two photographs depicting a mode of appending a device of the invention to the auricle, where the vibrating coin motor is in front of (medial to) the auricle (FIG. 14A), specifically located in the concha for maximal stimulation of the auricular branch of the vagus (CN 10), while the holding magnet is placed with the handle in back of (lateral to) auricle (FIG. 14B).

FIGS. 15A and 15B include two photographs depicting a mode of appending a device of the invention to the auricle. FIG. 15A shows placement of the device in the scaphoid fossa, a location which is useful in vibrating large areas of the auricular cartilage; stimulation in this location can quickly reduce anxiety and induce sleep. FIG. 15B shows how, if direct contact with the metal surfaces of the magnetic components is undesirable, intervening tissue can be placed with little or no loss of efficacy of the device.

FIGS. 16A and 16B include two anatomic charts depicting the optimal sites for vibratory patches for pain mediated by particular cranial and cervical nerves.

FIG. 17 includes a photograph depicting the composition of a snap connector.

FIGS. 18A, 18B and 18C include a series of three photographs depicting a motor combined with a snap connector for use with an adhesive patch.

FIGS. 19A and 19B include two photographs depicting a motor with snap connector and adhesive patch, for placement on multiple skin sites.

FIG. 20 includes a photograph depicting the gap outside the central snap element between the male and female part of a snap. The gap may or may not limit vibration transfer to an adhesive patch.

FIG. 21 is a schematic depicting a drawing of the vibrator motor, female and male snap connector components, optional vibration-distributing shell, and an adhesive patch. The vibration-distributing shell will assist vibration transfer in the area of the gap shown in FIG. 20.

FIG. 22 is a schematic depicting an exemplary embodiment of a device of the invention, including an optional surrounding plastic shell to further distribute vibration to the adhesive patch, where the secure and effective contact area between the vibration device and the patch is increased by a factor of 3 with the addition of the surrounding shell.

FIGS. 23A, 23B, 23C, 23D, 23E, and 23F include a series of photographs depicting construction details of a 12 mm diameter coin motor and snap connector within a circular plastic shell, and then snapped to an adhesive patch.

FIGS. 24A and 24B include an anatomic chart and a photograph depicting a vibrating patch placement over the intersection of V1 and V2 divisions of the trigeminal nerve. This placement is especially useful for nasal sinus pain and activation of parasympathetic fibers to enhance fluid expression.

FIGS. 25A and 25B include an anatomic chart and a photograph depicting an unusual representation of device placement to mediate activity of a nerve of the neck normally associated with motor, not sensory function (cervical nerve 1), which is easily accessed with a technique and device of the invention.

FIG. 26 is a chart depicting regional pain declines on a 10-point scale (10 most severe pain) in ratings for seven subjects. The Y axes indicate pain levels before and after intervention with the device, with pain levels defined under a 1-10 Pain Scale.

FIG. 27 is an anatomic chart depicting the corresponding sensory input from receptors of V1 and V2 regions to the brainstem.

FIGS. 28A and 28B includes two anatomic charts depicting the optimal sites for vibratory skin patches for pain behind the eye and lateral forehead pain.

FIG. 29 includes a photograph depicting the optimal placement of a vibration unit for attenuating pain in the lower neck and shoulder.

FIGS. 30A and 30B include two anatomic charts depicting cervical nerves C3 and C4 which form a plexus emerging just behind the sternocleidomastoid muscle on the lateral surface of the neck, which determines the optimal placement of a vibration unit for attenuating pain in the lower neck and shoulder. This figure should be viewed in the context of FIG. 29, which shows the vibrator placement.

FIG. 31 includes a photograph depicting the optimal placement of a vibration unit for pain sites in the upper areas of the neck, external ear, and posterior areas of the head.

FIGS. 32A and 32B include two anatomic charts depicting cervical nerves C2 and C3 which determines the optimal placement of a vibration unit for pain sites in higher regions of the neck, external ear, and posterior areas of the head.

FIG. 33 includes a photograph depicting placement over three sites, one overlapping V1 and V2 of the trigeminal nerve, just lateral to the left nostril, the second placed over the exit of the mandibular division of V3, just below the lower lip, and the third over the auriculotemporal nerve (near the anterior portion of the ear) of V3. These placements would be optimal for pain in the oral cavity, forehead pain, and impaired salivation in the oral cavity. The combined stimulation would also be optimal for preventing injury in ischemic stroke if vibration is applied within 3 hrs of stroke onset.

FIGS. 34A and 34B include two anatomic charts depicting trigeminal divisions V1, V2, and V3, which determines the optimal placement of vibration units for oral pain, salivation in oral regions, and stroke.

FIGS. 35A and 35B are an anatomic chart depicting vagal (cranial nerve 10) receptors on the auricle, relevant for migraine pain, pain in posterior oral cavity and upper pharynx, parasympathetic (cardiovascular, e.g., hypertension, atrial fibrillation, sleep, anxiety and visceral action); place device on the auricle near posterior concha.

FIGS. 36A and 36B show a small coupling device consisting of a flat magnet attached to a snap connector which will mate to an adhesive patch, providing a very small, and easily-detachable means to link a vibrator fitted to a flat magnet at the end of a cable (see FIG. 6), with an adhesive patch.

FIG. 37 is a chart depicting a decrease in systolic pressure in hypertensive subjects upon use of a device of the invention.

FIG. 38A is a side view of a nasal device and FIG. 38B is a magnified side view of a nasal device. FIG. 38C is a perspective view of the silicon component of a nasal device.

FIGS. 39A-39E are side views of target nerves for the nasal device.

FIG. 40 is a flow of participants through each stage of a study of effects of a vibratory device on proprioceptor fibers in premature infant breathing: enrolment, assignment, allocation, intervention exposure and analysis.

FIG. 41 is a table of demographic and neonatal characteristics.

FIGS. 42A and 42B show Respiratory traces (60 sec), from thoracoabdominal pressure sensors, in a 28 wks gestational age premature male infant (24 days old) (42A) at baseline, i.e., without vibratory proprioceptive stimulation and (42B) with proprioceptive stimulation. Fewer episodes of respiratory pauses, indicated by 4 arrows, occurred during the intervention, relative to baseline.

FIGS. 43A-43D are graphs of the sequence of events following a breathing pause in a 20-day-old premature infant (27 5/7 wks gestational age) showing (43A) breathing trace, (43B) oxygen saturation, (43C) electrocardiogram—ECG, and (43D) heart rate in beats per minute (bpm). In this premature infant, a 13 sec breathing pause (A) was followed by slowing of heart rate (43C), leading to bradycardia to <80 bpm (43D) and a desaturation episode (43B) to <90% lasting approximately 25 sec.

FIGS. 44A and 44B are graphs showing the effects of vibratory proprioceptive stimulation on a total number and duration of breathing pauses. As shown in FIG. 44A, proprioceptive stimulation significantly reduced the total number of long breathing pauses. As shown in FIG. 44B, proprioceptive stimulation significantly reduced the total duration of long breathing pauses. Mean and standard error from pre-transformed t-tests are presented for ease of interpretation. Measures are similarly presented for all of the comparisons below. * indicates p<0.05.

FIGS. 45A and 45B are graphs showing the effects of vibratory proprioceptive stimulation on a total number and duration of desaturations. As shown in FIG. 45A, during proprioceptive stimulation, premature infants experienced significantly fewer desaturation episodes, compared to no stimulation. As shown in FIG. 45B, proprioceptive stimulation significantly reduced the total duration of IH episodes as well. * indicates p<0.05.

FIGS. 46A and 46B are graphs showing the effects of proprioceptive stimulation on bradycardias. As shown in FIG. 46A, both mild (<110 bpm) and moderate (<100 bpm) bradycardia episodes were reduced by 3-fold during the stimulation period, compared to no-stimulation periods. As shown in FIG. 46B, a 3-fold reduction in the total duration of both mild and moderate bradycardia episodes also appeared with stimulation. * indicates p<0.05.

FIGS. 47A and 47B are nursing reports of apneas, bradycardias and desaturation cardiorespiratory events in their total (FIG. 47A) and duration (FIG. 47B).

FIGS. 48A and 48B are graphs of Respiratory and Mean arterial pressure (MAP) traces during a (FIG. 48A) 4 min baseline (no vibration) and (FIG. 48B) 2 min stimulation (vibration) period in a 28 weeks gestational age infant. FIGS. 48C and 48D are graphs showing fluctuations in systolic BP following apneic events (FIG. 48C) in a control subject not receiving vibrations and (FIG. 48D) in a treatment subject receiving vibrations.

FIGS. 49A and 49B are graphs showing diurnal trends of systolic BP (SBP) and diastolic BP (DBP) in (FIG. 49A) control subjects and (FIG. 49B) treatment subjects.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical devices, systems and methods for reducing headache and trigeminal neuropathy (oral-facial) pain. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### DESCRIPTION

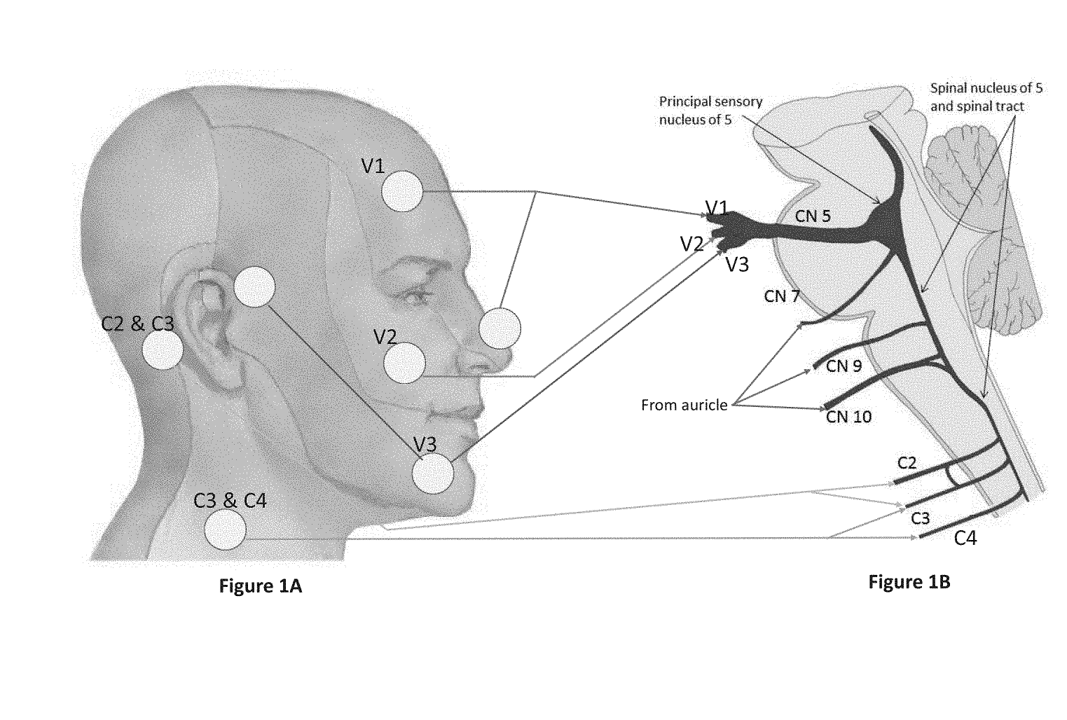

The devices, systems and methods of the invention make use of the potential to disrupt central nervous system processes that mediate pain by interrupting activity of pain neurons in a common brain area, the descending nucleus of the trigeminal nerve, as well as other nerves that carry other sensory information that can be used to “mask” pain signals. The sensory nerves which carry pain information from different areas of the head and neck send those signals to this common descending nucleus (FIG. 1). Overwhelming synchronous stimulation of fibers sensitive to pressure, touch, as well as pain, carry those signals to the descending nucleus, and to ventral posterior regions of the thalamus, from which integration of pain signals to the anterior cingulate cortex, insula, sensorimotor cortex, and the cerebellum take place. The input from vibratory signals would have the effect of disrupting the thalamocortical circuitry between the thalamic sites and cortical areas, thus diminishing pain (Henderson et al., 2013). In addition, particular cortical areas, especially the insular cortex, receives both pain and other somatic sensation, including information sensitive to vibration, from the descending nucleus of cranial nerve V and other brain afferent sites, and integrates these signals to reduce pain (Henderson et. al., 2007).

A focus of the current invention is pain of the head and surrounding areas, including the oral cavity, neck and shoulders, although other body areas may benefit. Other functions affected by vibratory stimulation may also be affected; these functions include movement control, including control of breathing musculature. The insular cortex serves substantial roles in depression and other mood disorders, and its activation by vibratory stimulation; half of the insula is devoted to somatosensory integration, and other portions to pain and blood pressure control.

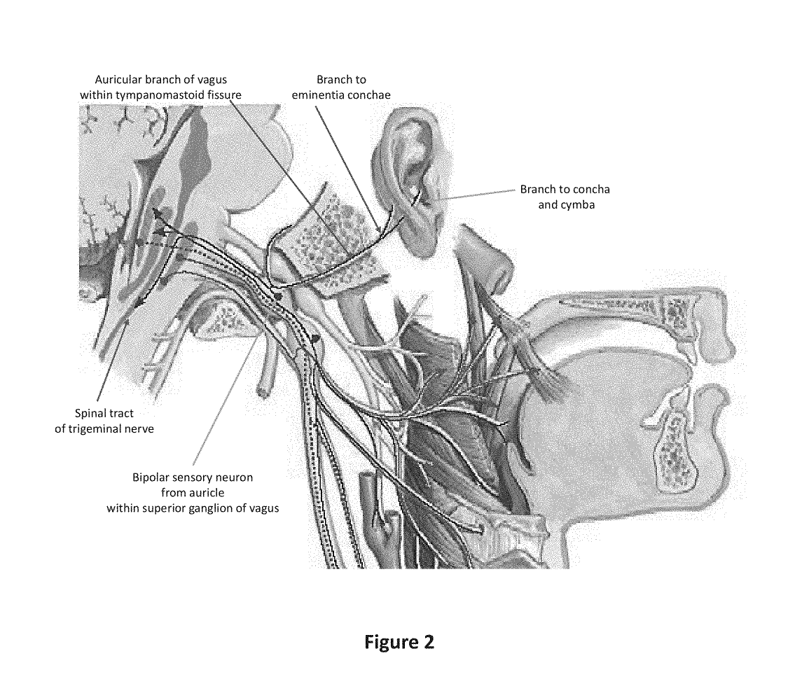

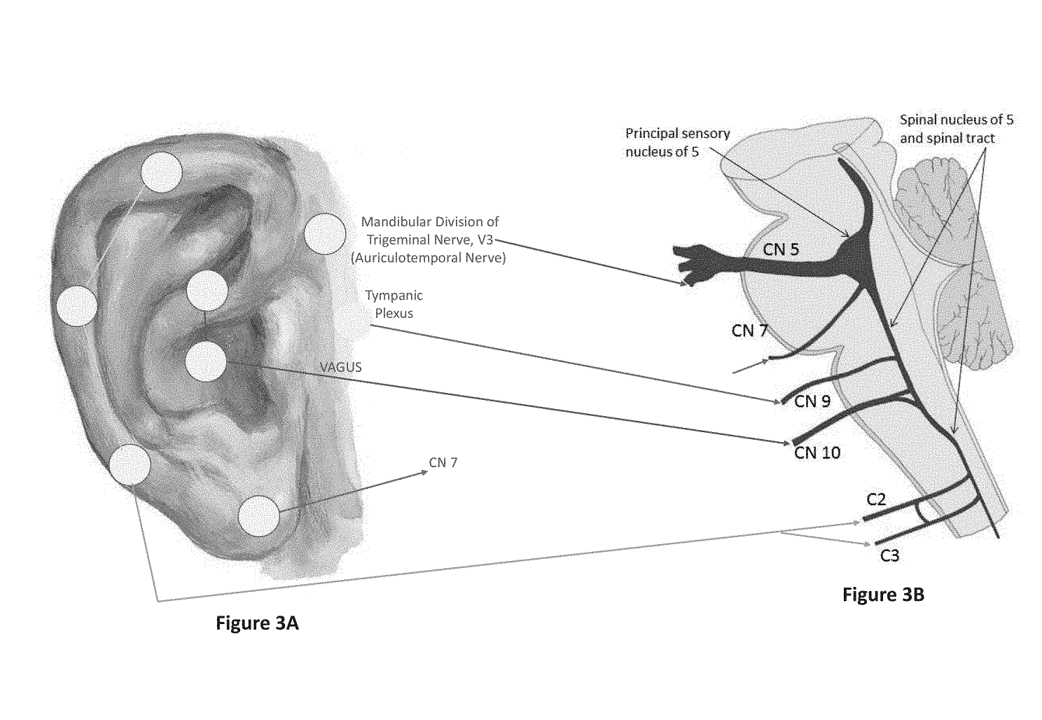

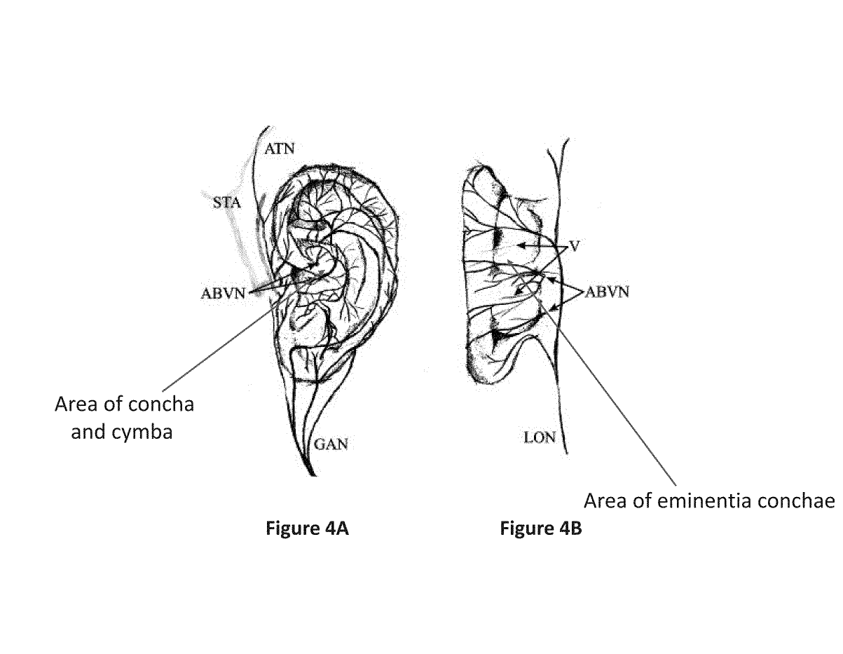

The procedures are unlike masking pain in the spinal cord which has been outlined earlier (Melzack and Wall, 1965), and which has been useful for intervention by electrical stimulation of spinal nerves. Much of cranial and oral pain is mediated by the 5th cranial (trigeminal, V) nerve, which serves the face, head, and dura covering the brain through three divisions (V₁, V₂, and V₃; FIG. 1), and integrates pain initially through the descending spinal nucleus of V. That nucleus also mediates pain from other cranial nerves, i.e., 7, 9, and 10, as well as from two cranial nerves of the posterior scalp, C2 and C3, and in a subset of patients, C1. It is important to note that a branch of the vagus nerve (cranial nerve 10), emerges through the skull as an auricular branch (FIG. 2), and serves areas of the external ear as well as the auditory meatus (FIGS. 3 and 4). The distribution of the vagus nerve to the external ear offers an opportunity to non-invasively stimulate that 10th cranial nerve, which has a very large distribution to the upper airway, heart, lungs, and viscera, and is considered a major influence on the parasympathetic component of the autonomic nervous system. Stimulation of the auricular branch of the vagus thus offers the potential to affect a large number of other physiological processes, including cardiovascular action, mediated by the vagus, as well as bronchioles of the lung, areas of the oral cavity and pharynx, and other viscera that may be subject to autonomic action or pain.

When placed over the sensory fields of the cranial and cervical nerves for the head, neck and shoulders, a vibratory device of the invention has the potential to disrupt on-going activity for multiple cranial and spinal nerves by interfering with transmission within the common pain projection, by masking pain signaling by overwhelming, faster-conducting mechanoreceptor nerve signaling, and, in the case of chronic pain, by interfering with thalamocortical circuitry known to be involved in long-term pain (the thalamus receives pain signals from the descending nucleus of V, and signals cortical areas which interpret the signals as painful). Similarly, when local vibration is applied to peripheral sites, such as adjacent to painful joint receptors, the vibratory stimuli to both pain fibers and to surrounding pressure and other mechanoreceptors can interfere with pain transmission pathways in the dorsal horn of the spinal cord. In addition, vibratory stimuli to lumbar and sacral representation on the periphery will interfere with sensory pain signals and disrupt activity of spinal and brain representation in pain integration areas. The device provides a compact, inexpensive means to provide non-invasive mechanical vibratory stimuli to nerves within the skin surface, and can be used for multiple conditions where activation of these primarily sensory nerves can alleviate conditions mediated by the brain. These conditions include migraine pain, regional pain in the head, neck, shoulders, limbs, or other areas of the body, or localized pain in joints of the leg or the feet.

A principal application of the invention is for pain relief, followed by interventions for correcting disturbed breathing, cardiovascular disorders and movement dysruptions. Regional pain from the nasal sinus regions or face, oral cavity, neck and shoulders, or from dura covering the brain or other areas involved in migraine is often debilitating. Localized pain in the lower back, limbs, especially in joints, often limits mobility. Devices have been developed to provide local electrical stimulation for relief of such pain, and are in common use (transcutaneous electrical stimulators, or TENS). Such electrical stimulation poses a risk for tissue injury at the site of electrical contact over long time use, and is a particular concern for sites on the face for obvious esthetic concerns for such injury. Moreover, electrical stimulation for such activation has the potential to elicit a number of different responses to the multiple types of cutaneous nerves, including sympathetic and other motor fibers, some of which are irritating to the subject. Finally, the Food and Drug Administration has proposed a ban on electrical stimulation devices used to treat self-injurious or aggressive behavior, since those devices present risks of a number of psychological injuries, including depression, anxiety, fear, and worsening of underlying symptoms, together with physical risks of pain, skin burns, and tissue damage. Such devices typically use higher currents than those employed for pain relief, but the ruling points out the risk of injury for electrical stimulation procedures.

The devices of the invention bring substantial relief of pain to a wide range of pain syndromes, and does so non-invasively and rapidly with minimal medical intervention after initial instruction. The intervention reduces pain within minutes of administration, typically 10-20 minutes, without use of pharmaceutical agents that may have deleterious cognitive, arousal, mood or motoric side effects. The device avoids use of paralytic muscle agents, such as Botox, or invasive surgery, e.g., lesions to cranial nerve nuclei to eliminate pain, or vascular decompression surgery to relieve blood vessel pressure from excitable nerves causing pain, all approaches currently used for trigeminal or migraine pain. Devices using electrical stimulation are currently used for pain emanating from spinal nerves as well as pain from the head. However, the use of electrical stimulation poses a risk of long-term application injuring the skin. The vibratory system of the invention poses no such risk. The device may also be used to “train” brain activity to reduce the incidence of epochs of headache pain, or to minimize the debilitating character of those headache episodes.

Vibratory stimuli as provided by the devices, systems and methods of the invention can also be used to activate cortical and other brain structures, thus inducing perfusion to the activated areas through reflexive vascular mechanisms, and reducing potential brain tissue injury resulting from stroke or other interference with the normal vascular supply. While invasive activation of cranial sensory nerves has been shown to improve mood conditions of depression and anxiety, as well as the incidence of epileptic discharge, and reduction in neural injury resulting from stroke, non-invasive cutaneous stimulation has the potential to provide similar relief without the potential for injury posed by invasive procedures.

Sleep-disordered breathing, including obstructive sleep apnea, central apnea, and periodic breathing affects 12% of the US population. Premature infants show substantial periodic breathing and apnea of prematurity, with very young premature infants universally showing such breathing disorders. A principal deficit in obstructive sleep apnea underlying muscle collapse in the condition is reduced sensory input from trigeminal (V), and other upper airway sensory nerves. We demonstrated the use of these vibratory devices on nerves of peripheral limbs to reduce apnea in premature infants, adolescent spinal cord patients (who hypoventilate during sleep), and adults. In addition, we provide a mechanism here through vibration of components of the nasal cavity a means to activate nerves of the oral cavity which will enhance sensory stimulation to upper airway muscles, overcoming airway obstruction in obstructive sleep apnea, and assist in timing of respiratory muscle action to prevent periodic breathing and central apnea.

Trigeminal nerve activation exerts powerful effects on the cardiovascular system, principally of a parasympathetic nature, which will normalize hypertension and hypotension, and reduce cardiac arrhythmias. The trigeminal effects are well-known from outcomes of cooling the forehead or pressure on the eyes; the vibratory effects of one embodiment of the procedure here, which stimulates nerves within the nasal cavity, provides a means to directly activate the trigeminal nerves and their accompanying parasympathetic fibers. The parasympathetic action will have a side benefit of providing relief from dry mouth syndrome, a typical accompaniment of oral pain.

### Device and System

The system and devices of the invention achieve cutaneous nerve activation without electrical stimulation by using mechanical vibration, which principally activates mechanical sensory receptors in the skin. The vibration is induced by a miniature coin motor, and conveyed to the skin either with a standard disposable patch electrode used for routine electrocardiographic (ECG) recording, or through a magnetically-coupled arrangement with the coin motor on one side of an appendage, such as the external ear, held in place magnetically with another magnet on the other side of tissue, such as the external ear. The devices of the invention apply vibration very near other sensory areas innervated by the cranial nerves which exit the skull, or spinal nerves exiting the spinal cord, to supply an area, and is not limited to cranial nerves supplying the ear canal. The devices address pain in portions of the head, such as near the nasal sinus, or in areas of the neck, shoulder, or limbs which may or may not be well-addressed by nerves of the auditory meatus. Delivery to the skin surface can be accomplished with slightly different variations in device form. The device and system of the present invention may be further described in light of and in reference to the accompanying Figures.

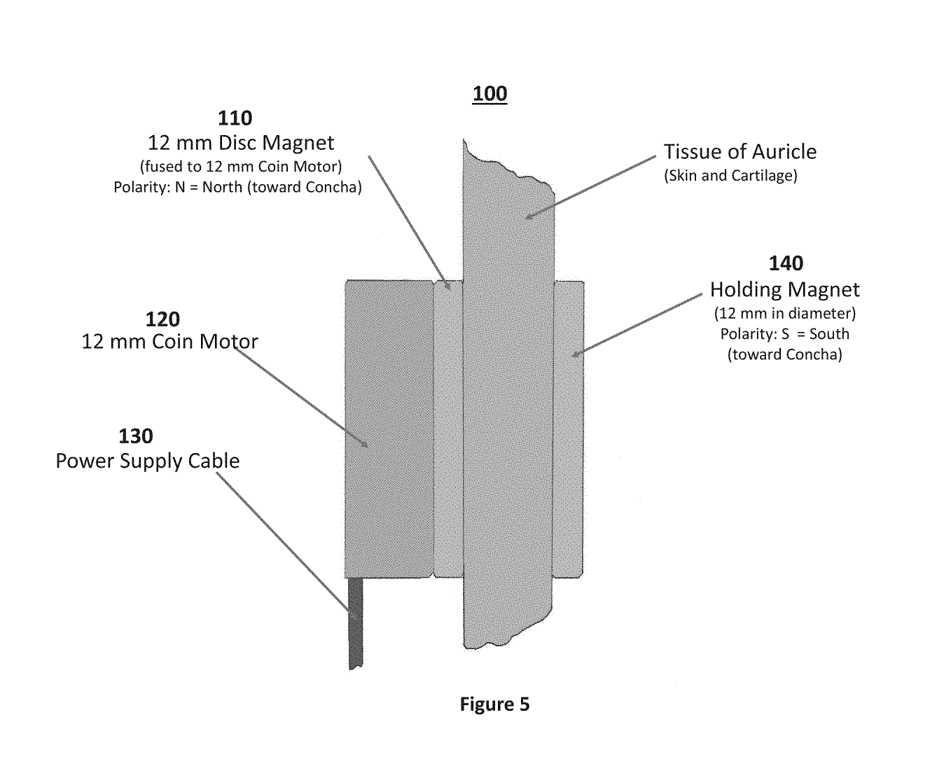

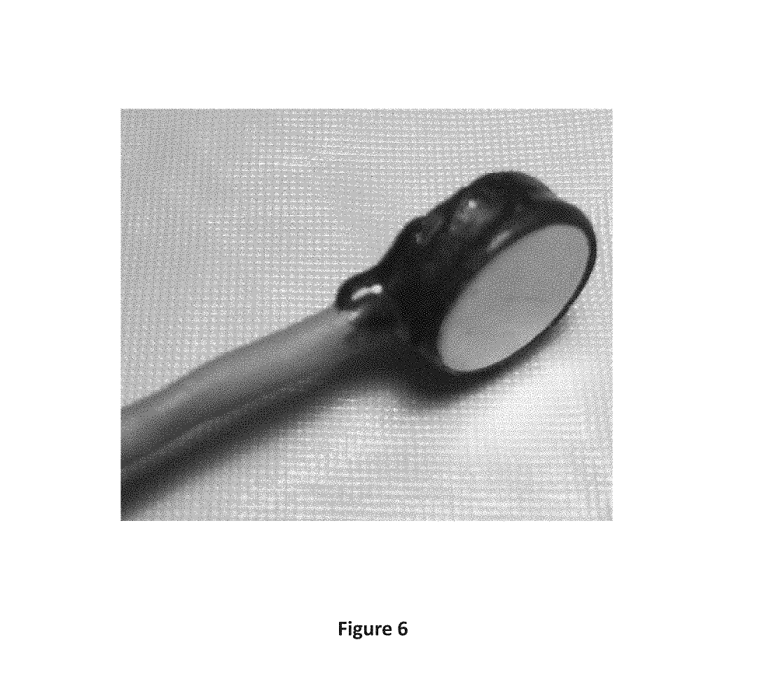

In one form, a coin motor, supplied by external battery power, is cemented to a disc magnet, and the combined unit placed over the surface of an appendage, such as the external ear (pinna). Referring now to FIG. 5, an exemplary embodiment of the system and device of the invention is device 100. The device includes a vibration source 120, which in one embodiment is a vibration motor. The device further includes a magnet 110, which is appended by cementing, gluing, or otherwise attaching it to the vibration source 120. The vibration source can be powered through a power supply cable 130, or can alternatively be powered by an onboard disposable or rechargeable battery. The vibration motor is held in place by another separate magnet 140. The holding magnet can be placed opposite to the magnet attached to the vibration source, such as for example on opposite sides of a tissue portion of a subject. In one embodiment the tissue can be part of the auricle of a subject, such as the pinna. The magnet attached to the vibration source and the separate holding magnet are releasably engaged and attracted to each other through magnetic field and are hold in place as a result. It should be appreciated that there are no limitations to the actual shape and/or dimensions of the vibration motor and the magnets.

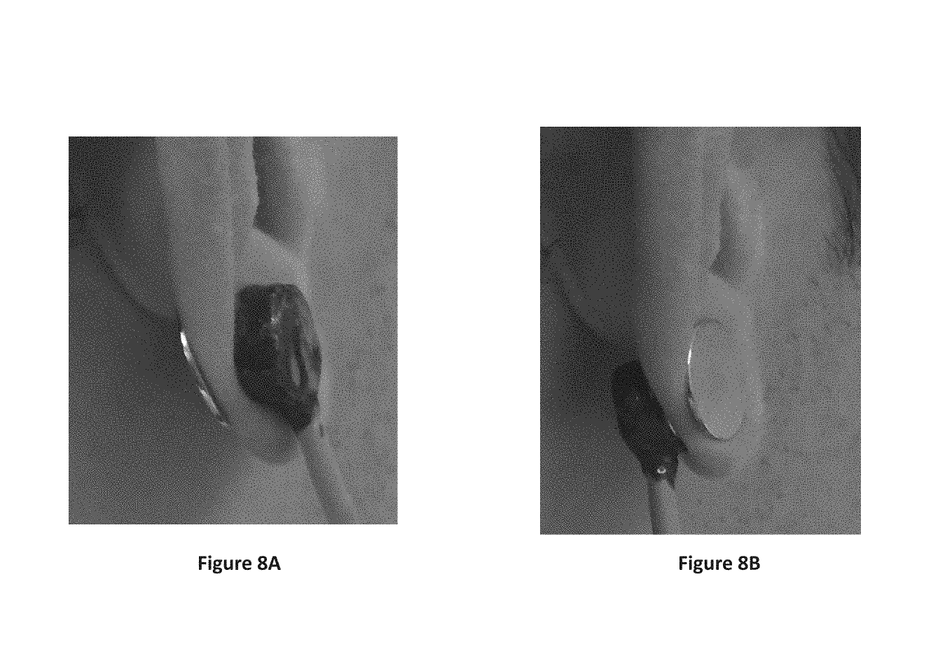

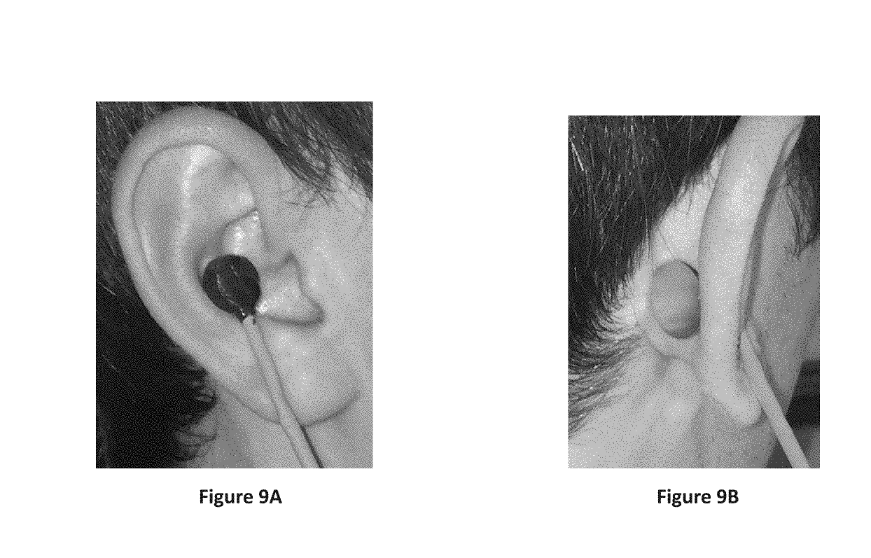

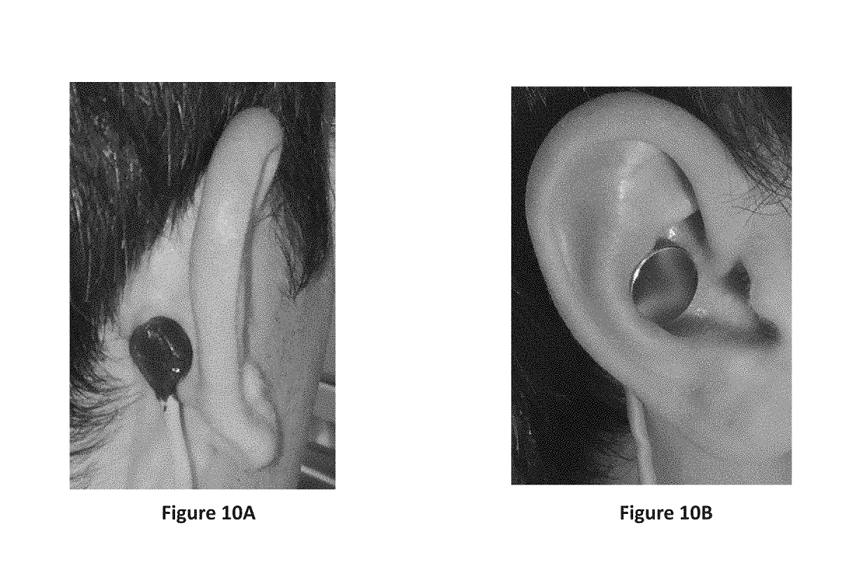

In other embodiments, the motor with fused magnet is placed on either the medial or lateral side of the pinna, and on the opposite side of the pinna is placed another matching magnet with polarity oriented to attract the magnet of the motor/magnet assembly on the other side of the pinna (FIGS. 8, 9, and 10). Vibratory stimulation of the pinna to affect sensory fibers of several cranial nerves, including cranial nerves 5 and 10, as well as cervical nerves 2 and 3, is useful for migraine pain, blood pressure regulation, mood remediation, induction of sleep, and epilepsy seizure reduction. Referring now to FIG. 8A, the vibrating coin motor is placed on the lateral surface of the auricle helix tail with a holding magnet on the medial side. The placement of the coin motor and holding magnet is reversed as shown in FIG. 8B, with the motor on the medial surface of auricle and the circular holding magnet on the lateral (external) surface. In other embodiments, the position of the vibrating coin motor is optimized to primarily affect select cranial nerve sensory fibers over others (FIG. 9), for example, by placing the vibrating device in the posterior concha, and holding it in place on the medial side of the auricle with the holding magnet. As shown in FIG. 10, the order of the vibrating motor and holding magnet can be reversed in more difficult access circumstances. For example, the coin motor can be placed on the auricle medial to the posterior border of the concha (FIG. 10A), with the holding magnet on the lateral (external) surface (FIG. 10B).

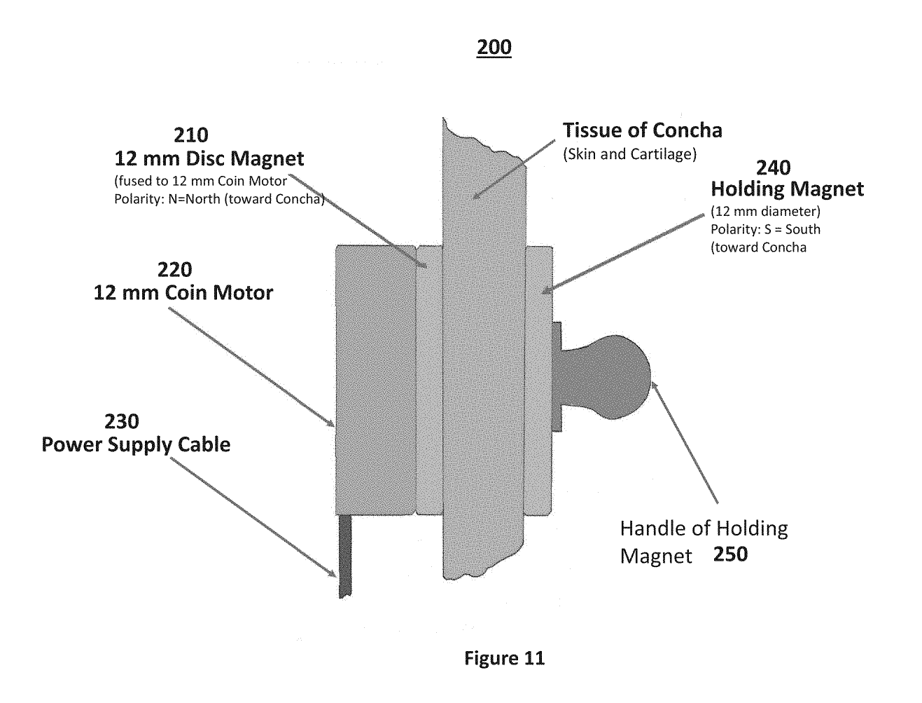

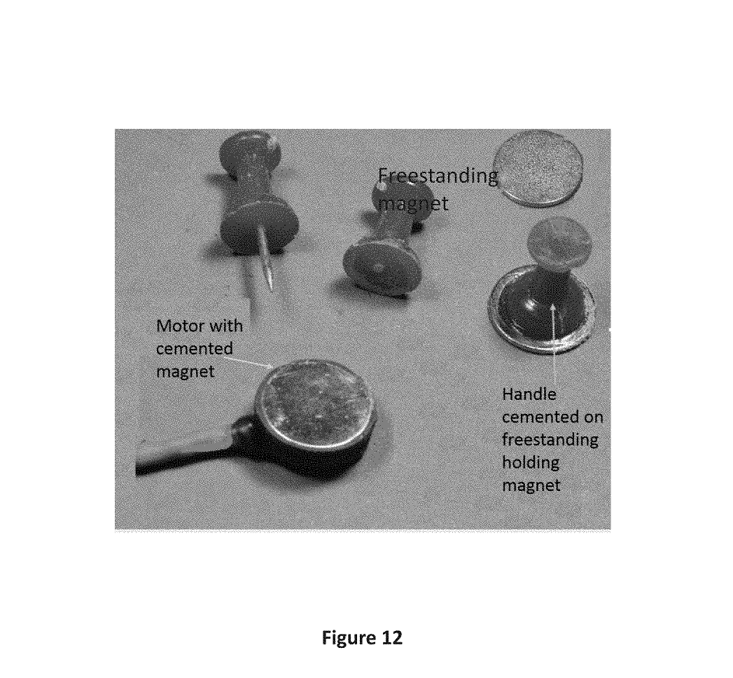

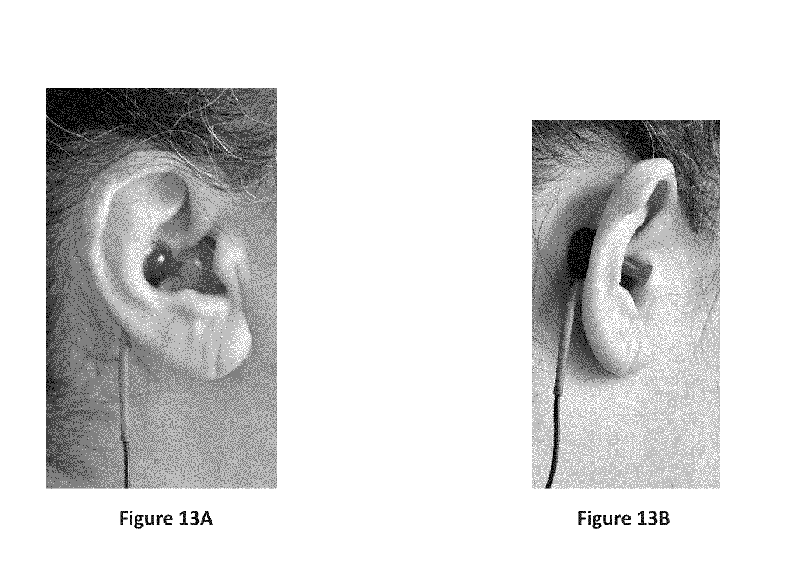

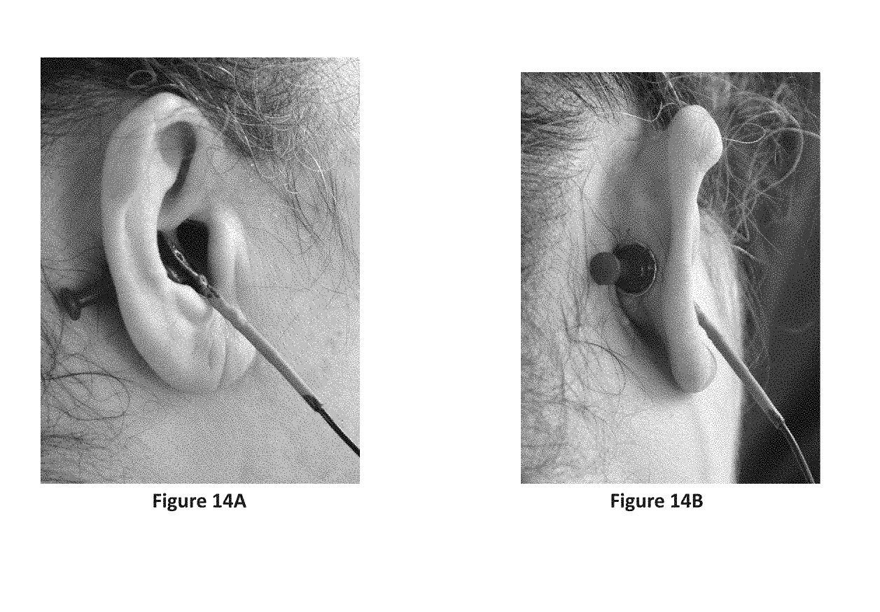

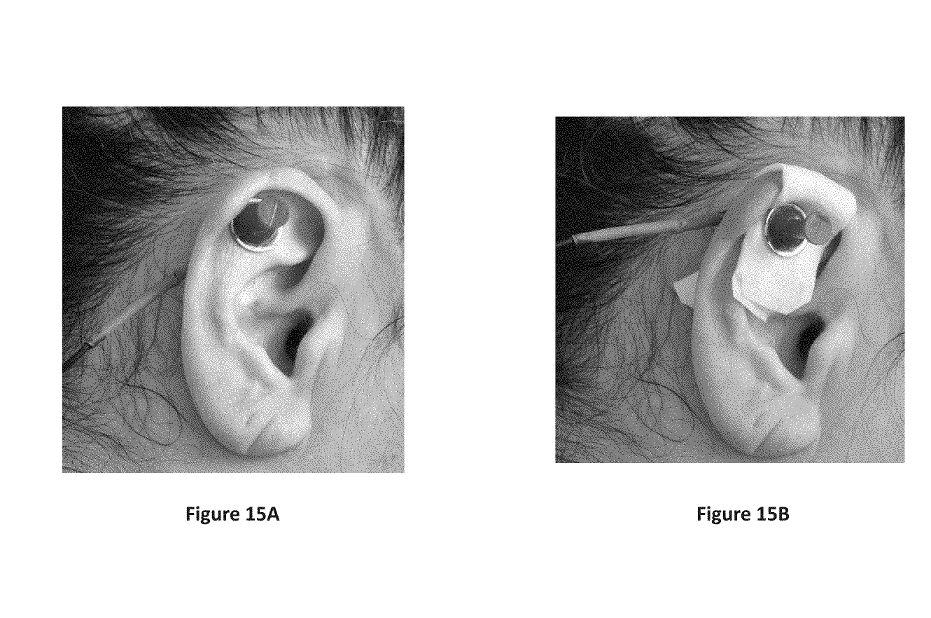

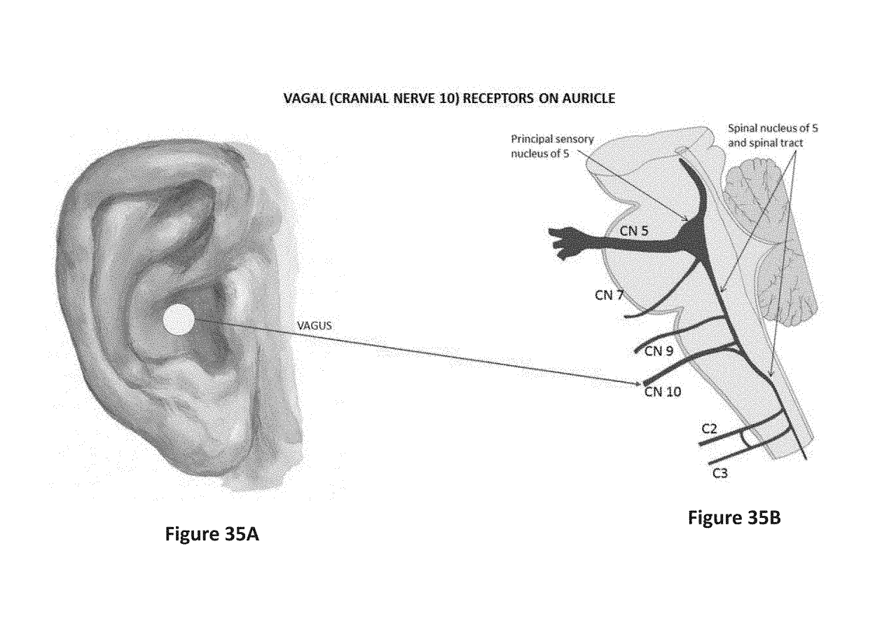

Referring now to FIG. 11, in one embodiment of the invention, i.e., the device 200, the separate holding magnet 240 can have a handle 250 to assist with placement (FIGS. 11-15). The handle can be made of a plastic material, or any suitable material (FIG. 12). It should be appreciated that there are no limitations to the actual shape and/or dimensions of the holding magnet and handle. In one embodiment of the device where the holding magnet has a handle, the device is placed on the back of the auricle, specifically located on the eminentia conchae for maximal stimulation of the auricular branch of the vagus (CN 10), while the holding magnet is placed with the handle in front of (lateral to) auricle (FIG. 13). The placement can be reversed, for example by placing the vibrating coin motor in front of (medial to) the auricle (FIG. 14A), specifically located in the concha for maximal stimulation of the auricular branch of the vagus (CN 10), while the holding magnet is placed with the handle in back of (lateral to) auricle (FIG. 14B). This placement is particularly beneficial for stimulating the vagus, i.e., cranial nerve 10, relevant for migraine pain, pain in posterior oral cavity and upper pharynx, parasympathetic stimulation (cardiovascular, sleep, anxiety and visceral action; FIG. 35). As shown in FIG. 15A, the device can be placed in the scaphoid fossa of the pinna, a location which is useful in vibrating large areas of the auricular cartilage; stimulation in this location can quickly reduce anxiety and induce sleep. FIG. 15B shows how, if direct contact with the metal surfaces of the magnetic components is undesirable, intervening tissue or any other suitable material can be placed with little or no loss of efficacy of the device.

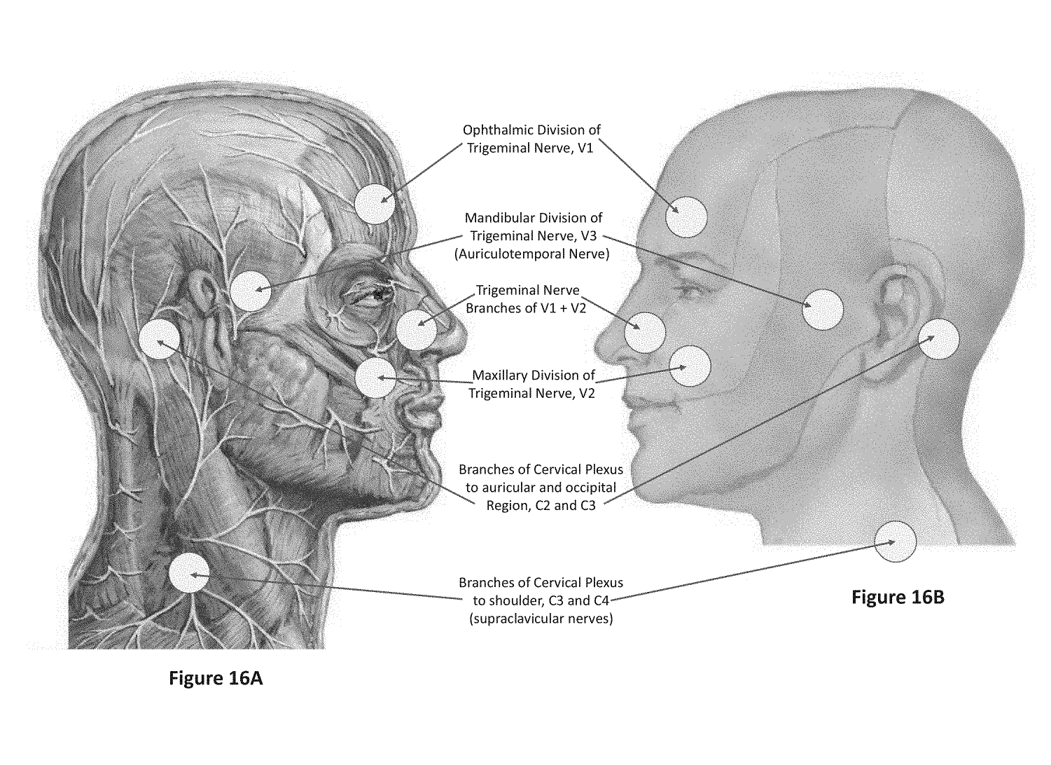

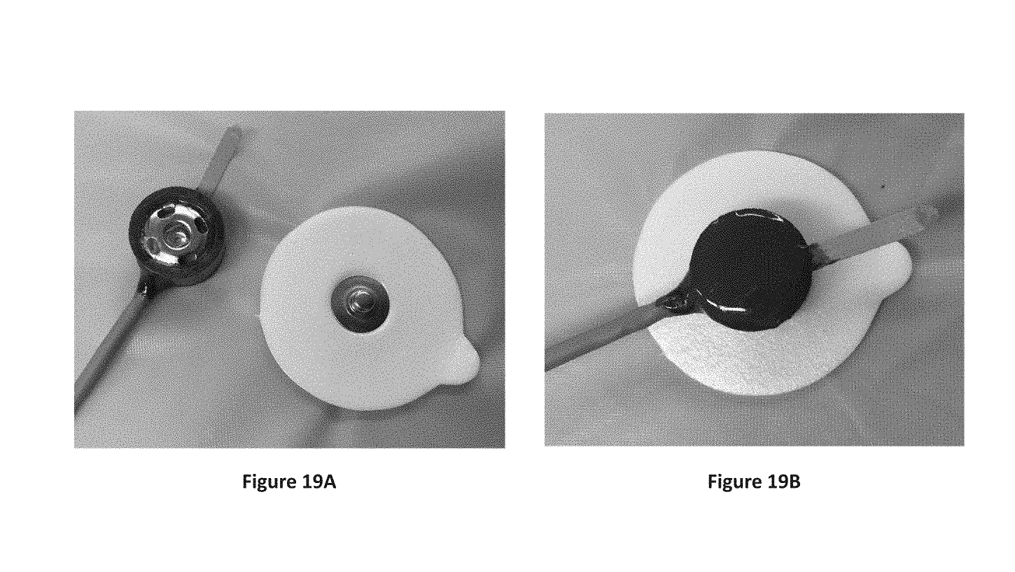

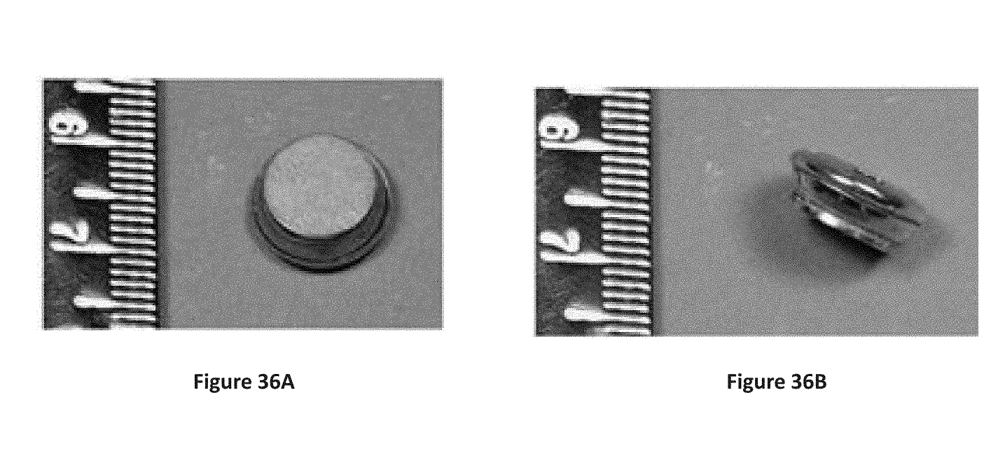

In another embodiment, a variation in device configuration allows the vibrating motor to be applied to local areas anywhere on the face or skull, as well as on the neck, shoulder, ankle or other localized regions of the body where the use of a separate holding magnet would be difficult or not feasible (FIG. 16), because the thickness of the intervening tissue is larger than the typical thickness of the auricle. In one embodiment, the device can use an adhesive patch having a magnet attached to it. The vibration motor and the magnet attached to it can be magnetically and releasably attached to the adhesive patch having the separate magnet, while the adhesive patch is placed on the skin, in the desired position. In certain embodiments, the magnet of the device and the magnet of the adhesive patch have opposite polarity. For example, in one embodiment, the magnet of the device has a default magnetic polarity of South while the magnet attached to the adhesive patch has a default magnetic polarity of North. When the two components are in close proximity, the two magnets automatically latch onto each other and establish a precise and firm, but releasable, connection between the device and the adhesive patch. Such an embodiment, illustrated in FIGS. 36A and 36B, results in an exceptionally small device, smaller than illustrated in FIGS. 19A and 19B, and has the advantage that the vibration device and the cable can be readily removed from the snap connector and patch by simply sliding the vibrating device with cable off the magnetically-coupled attachment to the snap connector on the electrode patch. It should be appreciated that there are no limitations to the actual shape and/or dimensions of the vibration motor, the magnets, and the adhesive patch.

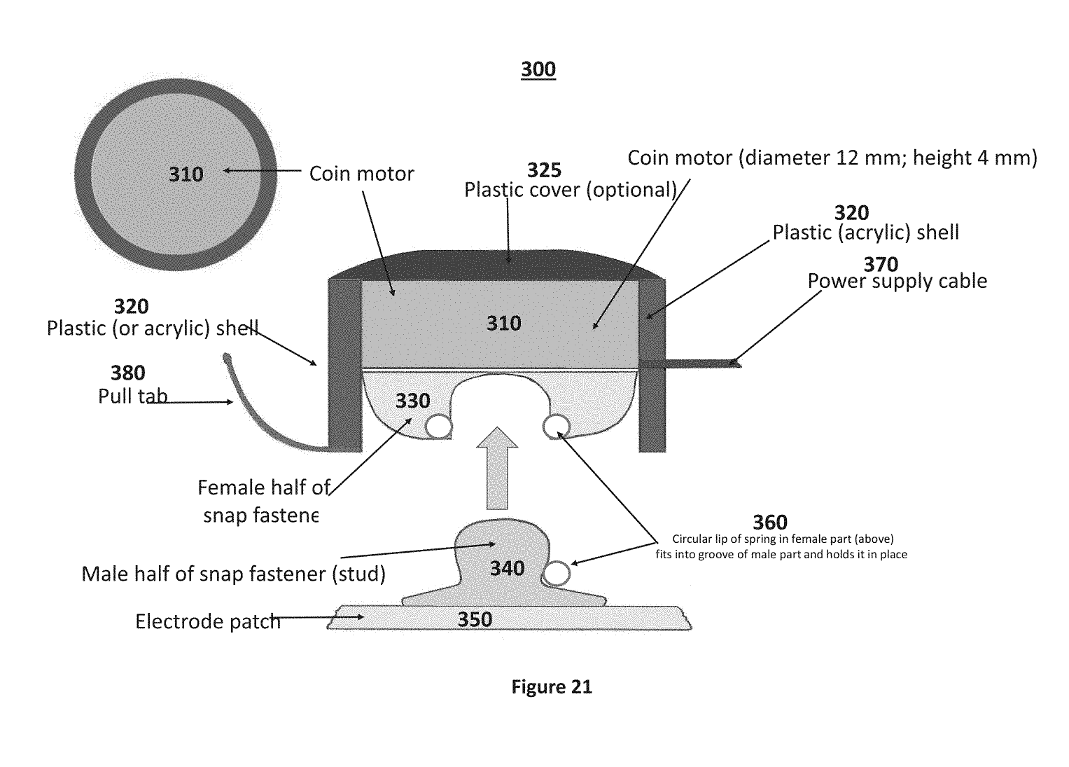

In another embodiment, the vibrating coin motor can be cemented for example to a female snap connector which connects to a conventional disposable adhesive patch, for example an electrocardiogram (ECG) patch electrode, having the male snap connector attached to it. Referring now to FIG. 21 depicting an exemplary embodiment device 300, the vibration source 310, for example a vibration motor, typically shaftless, can be attached to a mechanical connector 330. In this configuration, the device would be further releasably connected to an additional mechanical connector 340 which is attached to an adhesive patch 350. Mechanical connectors 330 and 340 have matching features allowing for attachment and detachment. Connector 330 for example can have a recess, while connector 340 can have an indentation, wherein the recess and the indentation have generally the same shape. The indentation can be placed in the recess and retained in place by a spring-loaded feature 360. The placement of the mechanical connectors can be reversed between the vibration motor and the adhesive patch, i.e., the vibration motor can be attached to a mechanical connector having an indentation, and the adhesive patch to a mechanical connector having a recess. The vibration motor can optionally be surrounded by a shell 320, for example a plastic shell. In one embodiment, the shell 320 achieves a better vibration transfer between the vibration motor and the adhesive patch 350, which results in better vibration transfer to the skin of the subject, and ultimately to the targeted nerve or portion of nerve. The device can be powered by an onboard battery, or through power supply cable 370. An optional pull tab 380 affords easier removal of the device. It should be appreciated that there are no limitations to the actual shape and/or dimensions of the vibration motor, the optional shell, the mechanical connectors, and the adhesive patch.

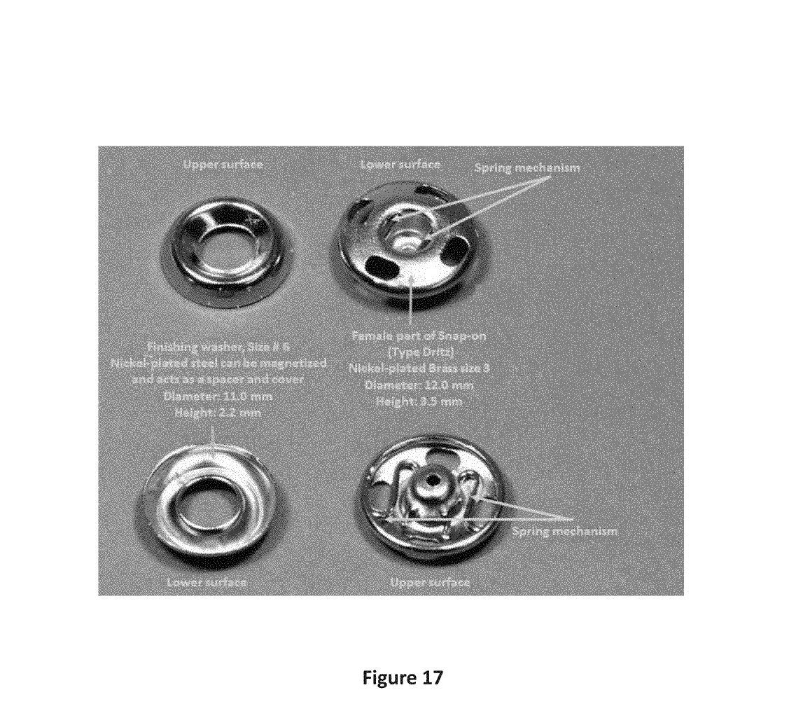

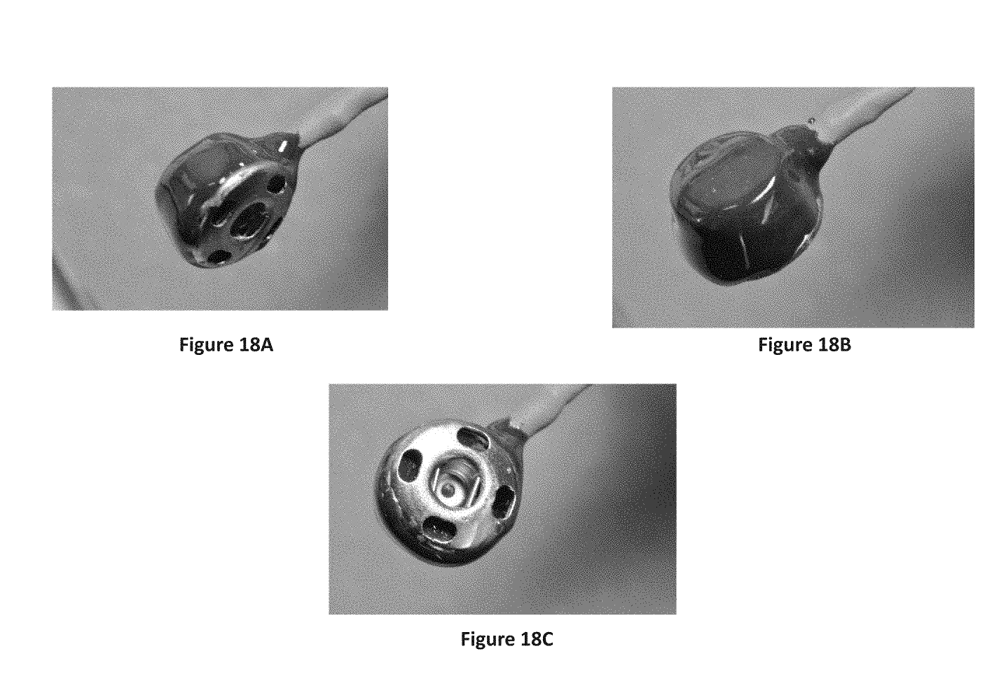

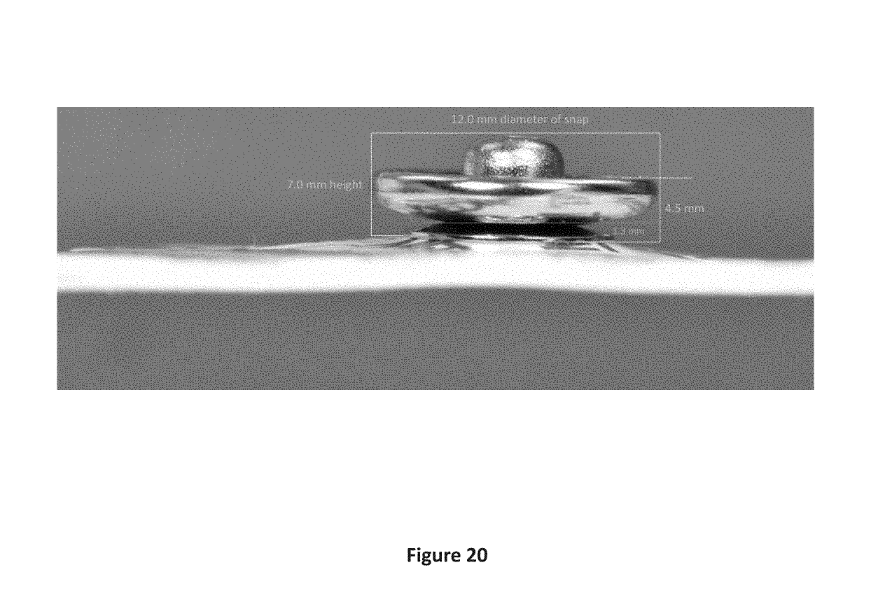

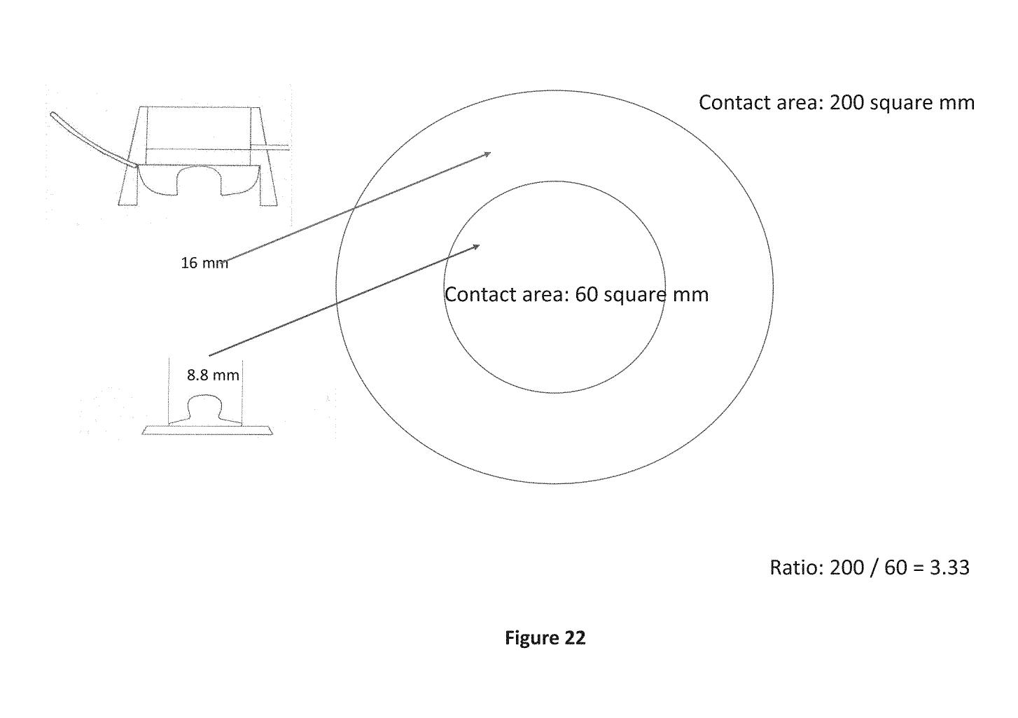

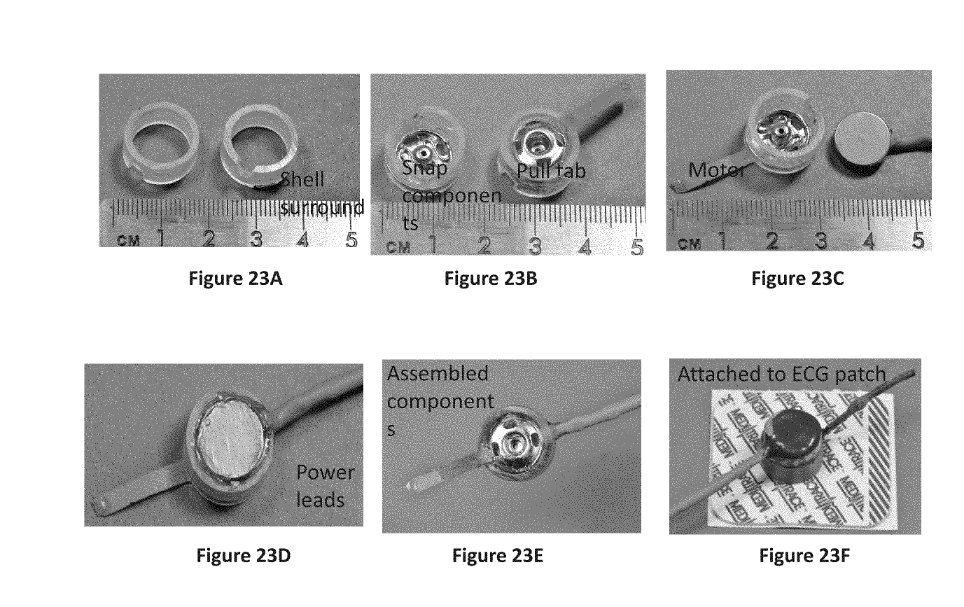

Exemplary components of the snap connector are shown in FIG. 17, the snap connectors being constructed from conventional millinery sources. A coin motor is cemented to a female snap connector (FIG. 18), which can then releasably be connected to a conventional male snap connector of an ECG patch (FIG. 19). The adhesive ECG patch is readily attached to most areas of the skin, is readily removed, and provides attachment with well-evaluated safety properties for skin application. A concern with the sole snap connector contact is that transmission of vibrations to the ECG patch is somewhat restricted with the small contact area of the female and male snap connectors (FIG. 20). For that reason, a variation of the device adds a plastic shell around the coin motor and female snap connector (FIGS. 19, 21, 22, and 23) to more effectively convey vibrations to the underlying male snap connector and patch.

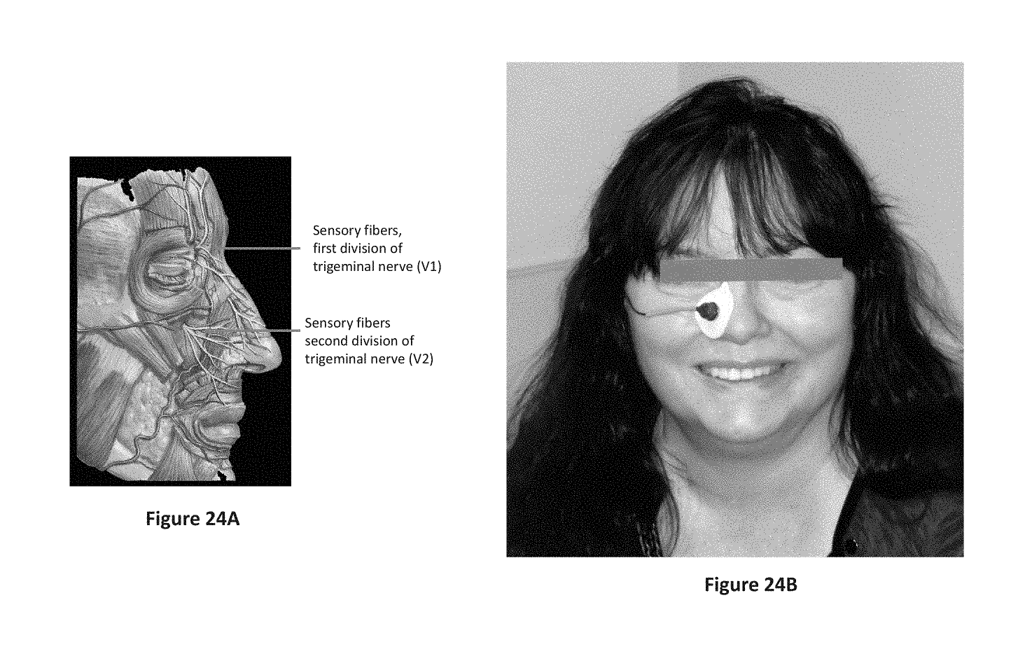

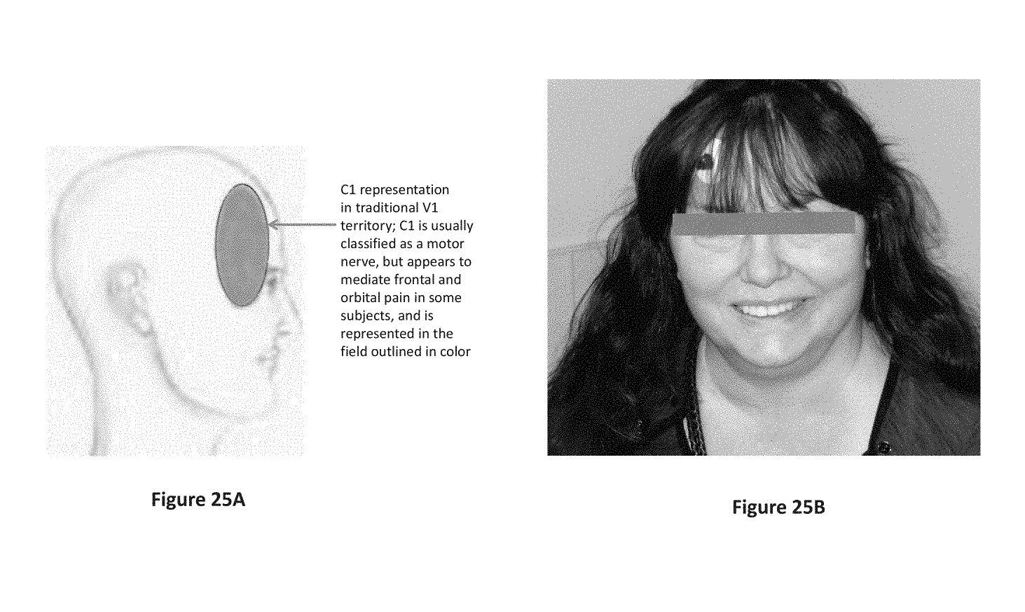

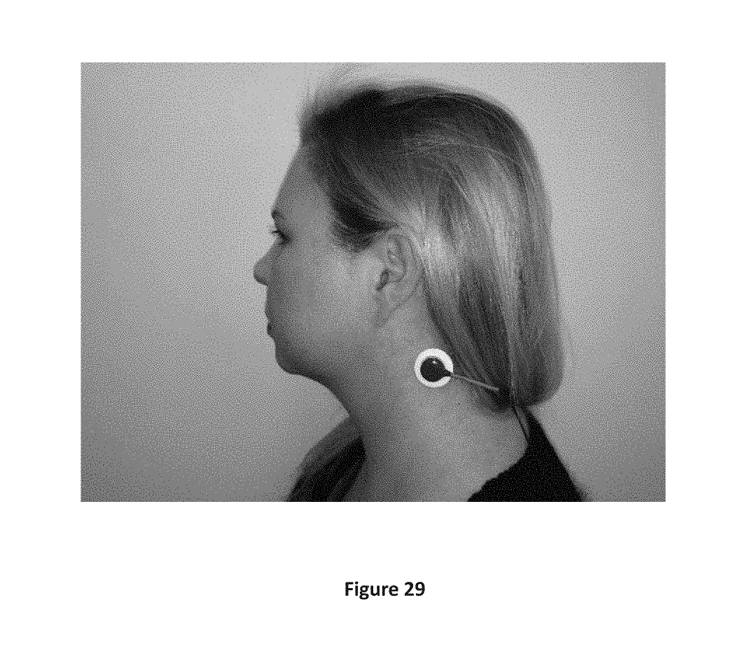

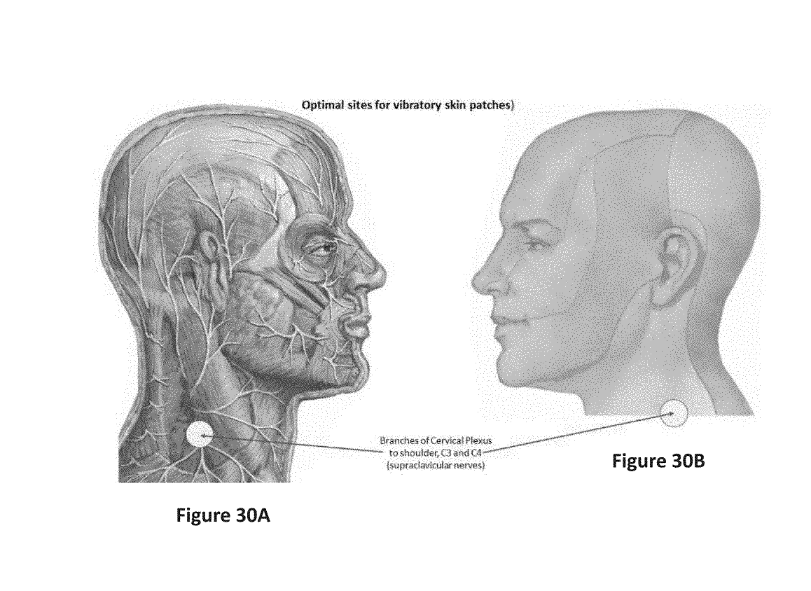

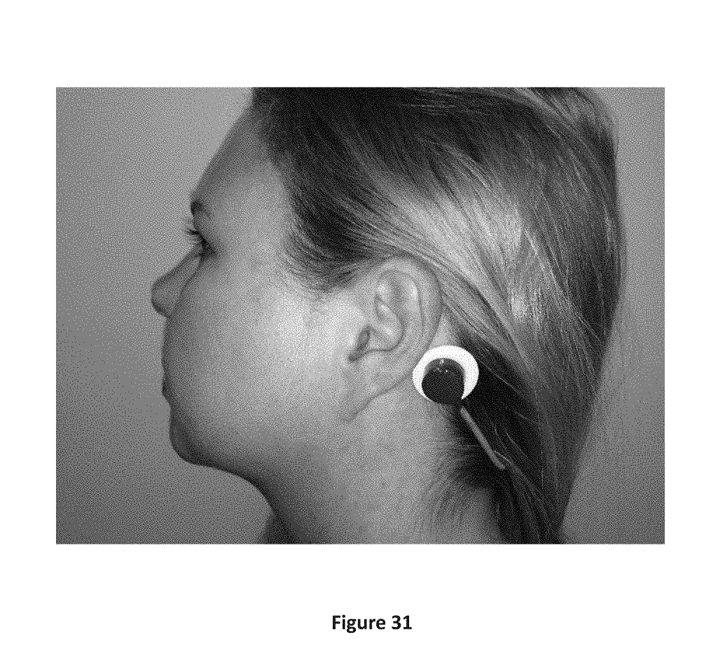

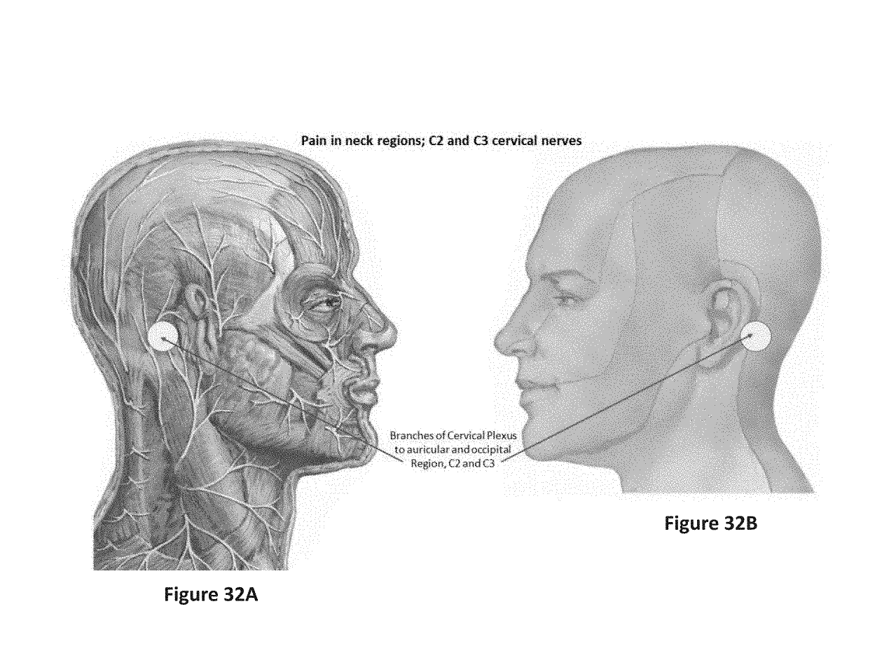

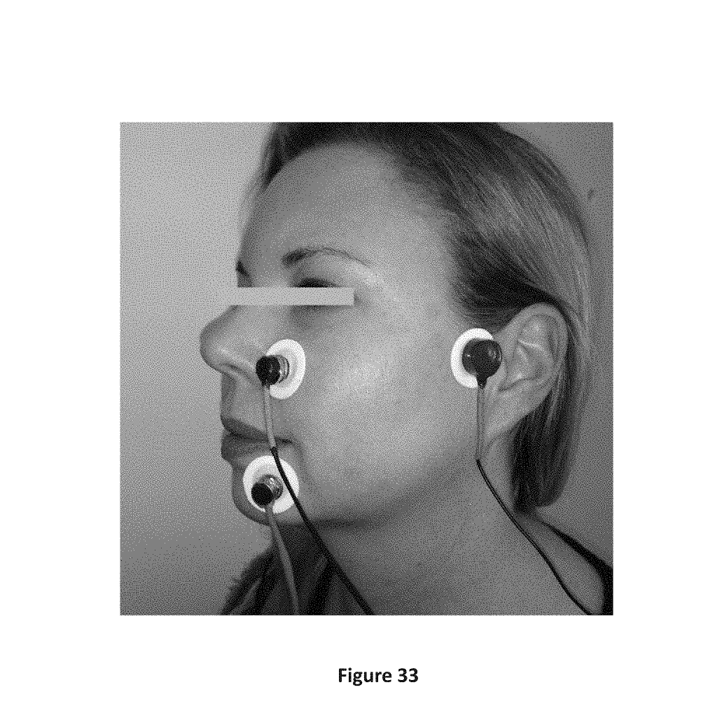

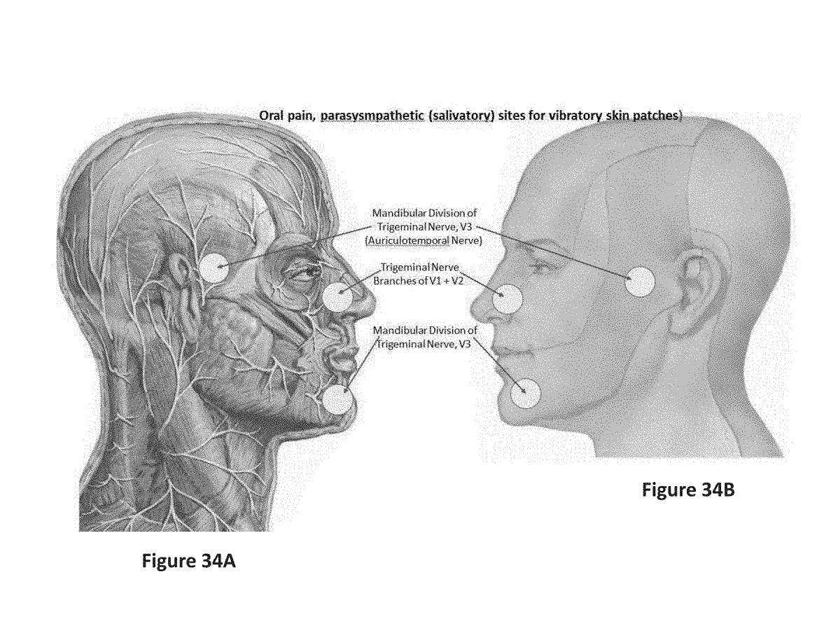

The patch, with attached vibration unit “snapped on,” is attached to the skin by the adhesive ECG patch close to the source of the cranial nerve mediating the pain or condition. In one embodiment, the patch is attached below the eye and lateral to the nasal opening near exit of infraorbital trigeminal cranial nerve V2, as well as components of V1, for sinus pain (FIG. 24). Both V1 and V2 divisions also carry parasympathetic motor fibers which supply glands and mucous tissue responsible for fluid release. Stimulation of the skin areas served by those divisions will elicit parasympathetic outflow, activating the glands and mucous tissue to secrete fluid, allowing the sinus to drain and relieve pressure. The fluid secretion also assists in patients with lack of salivation (dry mouth), a major concern in many oral pain syndromes. In another embodiment, the patch is placed immediately forward of the sternocleidomastoid muscle (C3 and C4), and behind the pinna of the ear (C2, C3) for shoulder, cervical (neck), and occipital pain, respectively (FIG. 16), or on the lateral forehead for pain mediated by the first division of the trigeminal (V1) and the first cervical (C1) nerve. The C1 nerve is often considered only a motor nerve, but has been recently demonstrated to have sensory components, with pain representation in unexpected regions (Johnston et al., 2013), including an area in the lateral forehead, normally considered to be innervated only by the first division of the trigeminal (FIG. 25). In another embodiment, the patch is placed just behind the sternocleidomastoid muscle on the lateral surface of the neck, where cervical nerves C3 and C4 form a plexus (FIGS. 29, 30). This placement will attenuate pain in the lower neck and shoulder. In another embodiment, the patch will be placed on the skin behind the ear, which will activate cervical nerves C2 and C3 for pain sites in the neck (FIGS. 31, 32). In another embodiment, patches can be placed on one or more sites, for example over three sites, one overlapping V1 and V2 of the trigeminal, just lateral to the opening of the nose, the second over the exit of the mandibular division of V3, just below the lower lip, and the third over the auriculotemporal nerve (near the anterior portion of the ear) of V3, placements which will attenuate oral pain, salivation in oral regions, and pain related to trigeminal divisions V1, V2, and V3 (FIGS. 33, 34).

In another embodiment, the patch device can be placed over regions of limbs that are sources of pain, such as the knee or ankle, or lower back.

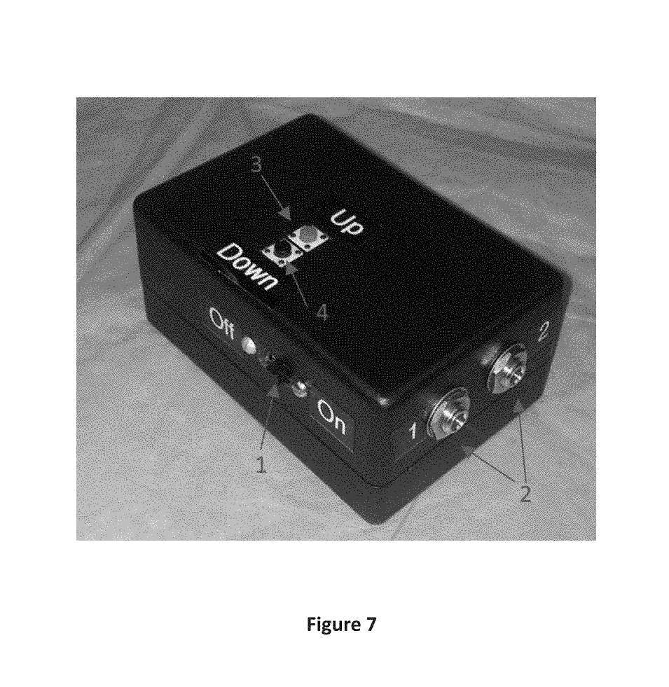

The vibration source, for example the vibration motor, can be powered through a cable from a stimulation box, or controller box, programmed through a computing device (FIG. 7). The controller can be designed and programmed to control the amplitude, timing, pulse train length, and interpulse interval of pulses for the vibration leads. In one embodiment, the controller has an On-Off switch (1), two output ports for vibrator leads (2), and two push buttons for fine adjustment of amplitude to the coin motors (3, amplitude up, and 4, amplitude down). Pulse characteristics are remotely programmed with a computing device, for example an Android device, and transmitted to the controller via wired or Bluetooth signals, where those parameters are stored within the memory of the controller, and optionally, stored on the computing device, for example a tablet, as well.

In one embodiment, the present invention provides a device and system comprising a computing device in communication with one or more of the control units, and/or vibration motors described elsewhere herein. For example, in one embodiment, one or more of the control units are programmed by a computing device, such as a remote desktop, laptop, smartphone, tablet, wearable computing device, and the like, which is in wired or in wireless communication with the control unit. The computing device may comprise software which may establish the amplitude, pulse rate, pulse burst duration, and interburst interval, and any other parameter of applied vibrational energy, as desired. In one embodiment, the computing device outputs a synchronizing signal to store on a recording device when concurrent physiological monitoring (necessary for those subjects who have concurrent autonomic pathology with pain). In certain embodiments, the computing device may be in direct communication, either via wired or wireless communication, with the vibration motor.

In one embodiment, the present invention may be controlled directly by a wireless computing device, such as tablets, smartphones or other wireless digital/cellular devices that are network enabled and include a software application platform or portal providing a user interface as contemplated herein. The applications platform may be a local or remotely executable software platform, or a hosted internet or network program or portal. The computing devices may include at least one processor, standard input and output devices, as well as all hardware and software typically found on computing devices for storing data and running programs, and for sending and receiving data over a network. The communications network between the computing device and the vibration source or component can be a wide area network and may be any suitable networked system understood by those having ordinary skill in the art, such as, for example, an open, wide area network (e.g., the internet), an electronic network, an optical network, a wireless network, personal area networks such as Bluetooth, a physically secure network or virtual private network, and any combinations thereof. In certain embodiments, the computing device comprises a display suitable for visual representation of system control and status. The communications between the computing device and the control unit and/or vibration motor may be conducted via any wireless based technology, including, but not limited to radio signals, near field communication systems, hypersonic signal, infrared systems, cellular signals, GSM, and the like.

In certain embodiments, the computing device comprises a software application used for the input of stimulation parameters, delivery of stimulation parameters, storage of stimulation protocols, storage of user information, and the like. The software application platform may be a local or remotely executable software platform, or a hosted internet or network program or portal.

The software platform includes a graphical user interface (GUI) for inputting stimulation parameters, modulating function of the control unit and vibration motor, and for displaying information regarding the historical or real-time functionality of the device, as well as historical or real-time pain perception. In certain embodiments, wireless communication for information transfer to and from the computing device may be via a wide area network and may form part of any suitable networked system understood by those having ordinary skill in the art for communication of data to additional computing devices, such as, for example, an open, wide area network (e.g., the internet), an electronic network, an optical network, a wireless network, personal area networks such as Bluetooth, a physically secure network or virtual private network, and any combinations thereof. Such an expanded network may also include any intermediate nodes, such as gateways, routers, bridges, internet service provider networks, public-switched telephone networks, proxy servers, firewalls, and the like, such that the network may be suitable for the transmission of information items and other data throughout the system.

As would be understood by those skilled in the art, the computing device may be wirelessly connected to the expanded network through, for example, a wireless modem, wireless router, wireless bridge, and the like. Additionally, the software platform of the system may utilize any conventional operating platform or combination of platforms (Windows, Mac OS, Unix, Linux, Android, etc.), and may utilize any conventional networking and communications software as would be understood by those skilled in the art.

To protect data, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access. Access or use restrictions may be implemented for users at any level. Such restrictions may include, for example, the assignment of user names and passwords that allow the use of the present invention, or the selection of one or more data types that the subservient user is allowed to view or manipulate.

In certain embodiments the network provides for telemetric data transfer to and from the control unit, vibration motor, and computing device. For example, data transfer can be made via any wireless communication technology, including, but not limited to radio signals, near field communication systems, hypersonic signal, infrared systems, cellular signals, GSM, and the like. In some embodiments, data transfer is conducted without the use of a specific network. Rather, in certain embodiments, data are directly transferred to and from the control unit and computing device via systems described above.

The software may include a software framework or architecture that optimizes ease of use of at least one existing software platform, and that may also extend the capabilities of at least one existing software platform. The software provides applications accessible to one or more users (e.g. patient, clinician, etc.) to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system. Presentation of data through the software may be in any sort and number of selectable formats. For example, a multi-layer format may be used, wherein additional information is available by viewing successively lower layers of presented information. Such layers may be made available by the use of drop down menus, tabbed pseudo manila folder files, or other layering techniques understood by those skilled in the art.

The software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text or phone call, to alert a patient, doctor, nurse, emergency medical technicians, or other health care provider of the particular results.

### Treatment Methods

The methods of the invention include procedures that use mechanical vibrations at particular frequencies optimized for mechanoreception in neurologic testing (128 Hz), and not electrical stimulation, to activate the underlying cutaneous sensory fibers, including those from cranial nerves, and surrounding mechanoreceptor nerves which can mask pain perception. The vibratory stimuli are non-invasive, and patient-controllable in intensity, frequency, and pulse pattern, with the patient adjusting the stimuli when pain appears. The patient may “condition” central nervous system (CNS) processes to suppress pain development, i.e., apply vibration to “train” the brain to suppress brain activity that might lead to later pain onset, wherein appropriate and effective mechanical vibration sites substantially reduce pain in multiple sites of the head, neck, shoulders and leg. Vibration can be initiated by the patient, and amplitude and pulse rate stimulation self-varied to minimize pain and maximize comfort. In patients with cervical (neck) and nasal sinus pain, resolution of pain once vibration is applied can be rapidly achieved (1-4 min). No additional pharmaceutical agents are used, and no electrical signals are applied to the body. The amplitude of vibration is in the range typically experienced by users of battery-powered toothbrushes, and do not pose any risk.

Placement on the skin of the subject, for example on the pinna of the ear, varies, depending on the particular portion of the vagus, trigeminal, or other cranial or cervical nerve to be stimulated. The tragus area (tissue just forward of the ear canal) is optimal for stimulation of the 3rd division of the trigeminal nerve (powerful cardiovascular, mandibular and oral pain effects). Upper posterior regions of the pinna are more appropriate for vagal (cranial nerve 10) stimulation. The device can be comfortably worn for long periods of time, and can be readily attached and removed without discomfort or injury to the tissue. Similar application to the cutaneous surface which contains sensory receptors for pain in the lower limb, such as the area lateral to the knee or ankle could be used for joint pain. Studies including subjects undergoing migraine, sinus, cervical, or limb pain show a substantial decline in pain, typically from a 5-7 range on a 10-point pain scale to 0 or 1 within 20 minutes.

Implementation of the intervention for any of the several conditions is normally performed under the direction of a medical professional skilled in diagnosing the source of a condition. For example, the source of head pain could stem from a tumor or skull fracture or some process where use of the patch system would only mask the underlying condition, and would be inappropriate to use. The intervention for ischemic stroke must be very rapid, but normally stroke can be diagnosed with a few simple tests by an emergency medical professional; in these cases the activation provided by the patch system could spare brain tissue that is rapidly lost in the wait for anticoagulant medications.

Implementation for pain relief will vary, depending on the source of pain. For nasal sinus pain, patches immediately lateral to the nares, covering V1 and V2 territory of the trigeminal nerve are appropriate (FIGS. 24A and 24B). For forehead pain, a patch located immediately above the eye, the exit for V₁ of the trigeminal nerve is most effective (FIG. 25). Use of the indwelling nasal device reaches both V₁ and V₂, and may more effective than external patches for V₁ and V₂. Pain in the cervical or shoulder region benefits by patch locations over C3-C4 exits immediately caudal to the sternocleidomastoid muscle (FIGS. 16A and 16B; FIGS. 30A and 30B). Pain within the knee benefits most from patches immediately above cutaneous sensory nerves to the knee. The patches are commercially available universally, and the snap vibrator units with cables, as well as the stimulation boxes, are very compact, and can be carried readily on the subject's person.

The ease of implementation, the innocuous nature, and the rapidity of determining efficacy of the device suggests that it be a first intervention before subjecting patients with regional pain to pharmaceutical agents or more invasive surgical means to block such pain. The device would typically be prescribed by a physician accredited to recognize and manage pain. The most appropriate implementation of the device is to use a portable package containing two units, one which contains the battery power, vibratory stimulation programming electronics, switches to establish settings of stimulus pulse duration, frequency and pattern, and a display screen to indicate appropriate stimulation characteristics. This unit will also contain an output jack for cables to the vibratory units. Once comfortable and effective vibration amplitude and rate levels are established in an initial trial with the patient, the patient will be sent home with the device to use when needed to reduce pain, or, with longer vibration periods, prevent occurrence of epochs of pain.

Ischemic stroke, typically brought about by occlusion of a major brain vessel by clot formation or by a severe vascular spasm, can elicit severe damage to brain tissue in the area normally perfused by that vessel, a consequence of tissue death by loss of the blood supply. However, studies in animal models (Lay and Frostig, 2014) show that if neurons in the region affected by the stroke are activated, the brain finds alternative means, presumably by very small vessels, to serve those activated neurons, a relationship familiar to those in the functional magnetic resonance imaging field. That process of induced perfusion will prevent long-term injury from an ischemic stroke. Animal studies show that, if the stroke occurs in the middle cerebral artery, an artery which serves large sensory and motor areas for the entire body, and vigorous stimulation of sensory fields is supplied by the fifth cranial nerve (trigeminal) within 3 hrs (in the animal models, whisker sensory fields; Lay and Frostig, 2014), the tissue surrounding the stroke is spared from injury. Those basic studies provide a remarkable potential to intervene in human stroke with the proposed vibrator device. The devices can be placed over trigeminal nerve areas on the face or within the nasal cavity which project to neural tissue served by the middle cerebral artery (frequently a casualty in human stroke, because of the unique path of the middle cerebral artery, with significant angular turns in humans, resulting in easy potential to deposit plaque, impeding blood flow). Ischemic stroke in those areas leads to severe motor and sensory deficits. Vibration will activate those brain areas; the trigeminal system serves very large areas of the brain in addition to primary sensory and motor areas; these areas include the insular cortices and basal ganglia, also major targets for stroke and areas very much involved in depression and autonomic control. Activation of sensory fibers for other brain areas should also be of benefit for stroke in those areas; cervical stimulation would be useful for cerebellar stroke.

Intervention for stroke requires rapid application; in animal models, the intervention must occur within the first three hours of stroke onset. The devices are sufficiently compact to fit within standard first aid or emergency kits, and implementation is simple application of the patches to the area of the face served by cranial nerve 5, divisions 1 and 2; the optimal position is immediately lateral to the nares on both sides of the nose (FIG. 24, and FIG. 33). If more time is available (about 10 minutes), a indwelling nasal device can be used. Those areas supply a large part of the dura and brain surface, and the neural tissue responsive to the most common stroke region, areas served by the middle cerebral artery. Other brain areas, such as the cerebellum or brain stem would be most effectively served by patches placed over the mid cervical area, just posterior to the sternocleidomastoid muscle (FIG. 16).

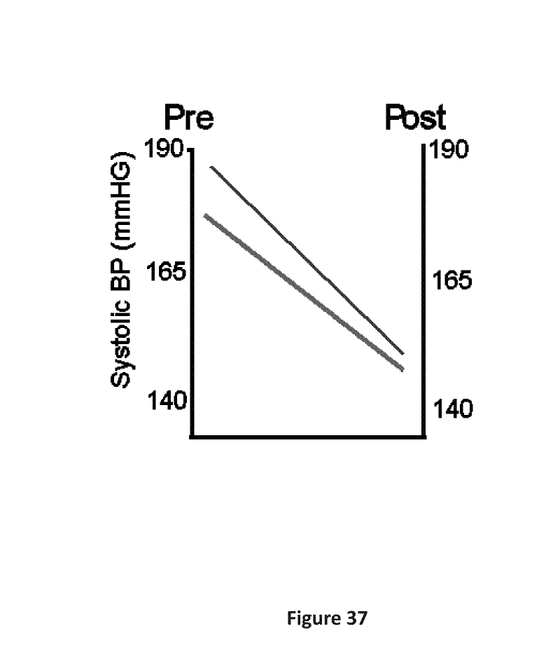

A range of cardiovascular, mood, and epilepsy aspects have been assisted by stimulation of sensory roots of the vagus nerve (cranial nerve 10); similar effects can be obtained through stimulation of trigeminal (V) nerve roots. The beneficial effects include reduction of blood pressure (Petkovich et al., 2015), correction of arrhythmia, especially atrial fibrillation (Stavrakis et al., 2015a), improvement in depression (Nahas et al., 2005), and reduction in frequency of epileptic seizures (Meng et al., 2015; Cukiert, 2015). Those effects, however, typically have been achieved by invasive exposure of the vagus nerve through surgery, placement of stimulation electrodes around the nerve, and leaving an implanted pacemaker unit within the body (leaving a potential source for infection). Despite the focus on invasive means to stimulate the vagus within the neck, that nerve extends to the skin surface as a sensory, cutaneous component, the auricular nerve, which arises through the temporal bone near the mastoid process posterior to the pinna of the ear (FIG. 2), and provides sensory innervation over large areas of the external ear (FIG. 4). That presence allows stimulation of sensory components of the vagus, avoiding deleterious consequences of directly activating motor components of the nerve bundle. Electrical vagal stimulation has been used to reduce atrial fibrillation (Yu et al., 2013; Stravrakis et al., 2015b), and to lower blood pressure (White et al., 2016). The cutaneous vagal components are readily accessible with the non-electrical, vibratory patch devices proposed, and lower systolic pressure in hypertensive subjects substantially (FIG. 37). Stimulation of the trigeminal nerve can achieve similar effects; the trigeminal system is much more accessible, and exerts powerful cardiac slowing and anti-arrhythmia actions.

The devices and methods of the invention can be used in cases of atrial fibrillation and abnormal blood pressure, for example hypertension or hypotension. Procedures to activate vagal fibers of the auricle by electrical stimulation for both reducing atrial fibrillation and lowering sympathetic nervous system outflow (the latter leading to reduced blood pressure have been described (Stavrakis et al., 2015; Clancy et al., 2014). Trigeminal stimulation will have similar effects. Evidence that electrical auricular nerve stimulation can alter activity in the principal nucleus mediating blood pressure control in the brain stem, the nucleus of the solitary tract, has been shown by magnetic resonance imaging techniques (Frangos et al., 2015). The vibratory device removes the substantial concerns for electrical stimulation to injure cutaneous tissue, since such stimulation may have to be carried out for long time periods. Once the presence of atrial fibrillation or hypertension is detected by an electrocardiogram or blood pressure reading by a cardiologist or physician, an intervention could be attempted immediately within the physician's office to determine effectiveness of the device. The patches would be placed over the exit of the trigeminal nerve over the forehead (V1), or branch exits on either side of the nose (V2), or within the nasal cavity (V1 and V2). Vagal cutaneous auricular branches which emerge through a tiny fissure near the mastoid process immediately posterior to the external ear on both sides of the head (FIG. 2) can also be used. The nerve is readily accessed on the surface of the external ear by placing an electrode over the antihelix (FIG. 3), and turned on for a period of time, for example, 30 minutes. A second electrocardiogram and blood pressure reading could then establish the presence of absence of atrial fibrillation after any of these applications. The patient could then be directed to use the device routinely at a schedule to be determined by the cardiologist.

Selective activation of sensory components has the potential to relieve bronchial constriction in chronic obstructive pulmonary disease (COPD) and asthma by reflexly activating sympathetic efferents, an approach taken by others with invasive techniques (Hoffmann et al., 2009), and local electrical stimulation of external fibers (Miner et al., 2012).

The accessibility of cutaneous sensory components of the upper airway, including the 10th, 9th, 7th, and 5th nerves to the vibratory device offers the potential to maintain upper airway muscle tone. Those nerves all provides sensory innervation to portions of the oral cavity and upper airway. Activation of those nerves is necessary to maintain muscle tone to upper airway muscles. Those muscles, especially muscles of the tongue (Sauerland and Harper, 1976), dilate the airway with each breath, and if flaccid, collapse during attempted breathing efforts of obstructive sleep apnea, blocking the airway so that no air exchange ensues despite continued movement of the diaphragm. The muscle tone collapse, or flaccidity, principally develops from a loss of sufficient stimulation from airflow and other sensory receptors, such as mechanoreceptors, to maintain motor output to the principal muscles dilating the airway for air exchange. The sensory stimulation that is lost includes information from principally the 5th (trigeminal) nerve, easily accessed with the nasal and facial devices here, but also includes receptors from the posterior oral airway encompassing territory of the 9th and 10th cranial nerves. Adequate stimulation to the oral airway can be provided by the external cutaneous vibratory devices, since the central nervous system is poor in discriminating the precise location of sensory information (e.g., pain from a myocardial infarction is often interpreted as pain in the arm), and thus the central nervous system will use that external cutaneous stimulation to maintain background airway muscle tone, thus preventing airway collapse. Another form of sleep disordered breathing is periodic, or Cheyne-Stokes breathing, repetitive breathing movements followed by a period, often lasting 30 sec or more, of cessation of breathing, which results in dangerous successive epochs of intermittent low oxygen. That breathing pattern is typically caused by a mismatch of timing in sensing of CO₂ in the carotid bodies of the neck vascular with central chemoreceptors in the brain. That breathing patterning can be overcome by vibratory stimulation to the 9th and 10th nerves, and thus can be managed by appropriate vibratory stimulation using the patch device.

The devices and methods of the invention can be used in sleep disordered breathing. The device will be of use for both obstructive sleep apnea and central apnea, where central apnea includes both sustained suppression of all respiratory muscles during sleep, and periodic, or Cheyne-Stokes breathing. In these cases, after disordered breathing has been determined by a physician who has overseen an all-night polysomnographic recording, devices will be placed in a nasal cavity or over the facial exit of V1, V2, and V3. Alternatively, devices in the external ear, just posterior to the concha for the auricular nerve, a branch of cranial nerve 10, V₁, V₂, and V₃ regions of the trigeminal, and C2-C3 regions of the neck, with very low level vibration applied to establish tone to the upper airway muscle motor pools. A second polysomnographic recording will be required to establish validity of the outcome.

The ability to activate cranial nerves 5, 7, and 10, and spinal nerves C1, C2, C3, and C4 provides a potential to significantly impact breathing pathologies, including three common sources of respiratory deficiencies, obstructive sleep apnea, periodic breathing, and hypoventilation. That potential stems from the capability to modify activity in nerves that can markedly modify breathing. Motor components of spinal nerve C3 and C4 form part of the phrenic nerve output, the principal nerve to the diaphragm, and can thus enhance hypoventilation, a major concern in spinal cord injury during sleep, in some genetic syndromes, such as congenital central hypoventilation, which shows a cessation of breathing during sleep, and a range of other breathing pathologies during sleep. Activation of cranial nerve 5, 7, and 10 can simulate airflow, and that afferent activity can reflexly activate upper airway motor drive to break upper airway obstruction in obstructive sleep apnea. Cranial nerve 10 provides sensory signals to cardiac motor and afferent receptors and pulmonary receptors, all critical in regulating timing of blood pressure and thoracic pressure regulation, and essential components in control of mechanisms underlying obstructive sleep apnea and central apnea. Other breathing patterns do not meet the usual definition of obstructed breathing, periodic breathing, or hypoventilation. A patient with Multiple Systems Atrophy, for example, in addition to obstructive sleep apnea and hypoventilation, also shows stridor, a characteristic resulting from failed action of the posterior cricoarytenoid (PCA) vocal cord dilators (or hyperactivity of the opposing laryngeal closure muscles). Such failures requires programming of cranial nerve 10 motor fibers to those muscles.

Other breathing conditions amenable to peripheral vagal stimulation is chronic obstructive pulmonary disease (COPD) and asthma, both of which are associated with constriction of the bronchioles of the lung. That constriction is relieved by the sympathetic nervous system (thus use of sympathetic agonists as inhalers for asthma), not parasympathetic fibers, such as cranial nerve 10. However, a reflexive action exists with stimulation of the afferent (sensory) fibers of cranial nerve 10, auricular branches of which are represented on the external ear. That reflexive action operates centrally to enhance sympathetic output. Thus, simple vibration to the external ear over regions served by the auricular branch of the vagus could enhance sympathetic drive to the bronchioles, relieving constriction and assisting air exchange. The trigeminal nerve complex also can exert profound parasympathetic influences that will balance sympathetic tone.

The devices and methods of the invention can be used for “retraining of breathing.” Long term use of the device can “retrain” appropriate breathing patterns by providing appropriate sensory stimuli to cerebellar and brainstem respiratory motor regulatory areas. Repetition of sensory stimulation will cause “relearning” of normal sensorimotor integrative processes to facilitate normal neural function. The goal is thus to provide initial respiratory “training” with the device, with the objective that after initial training, the device can be removed, with perhaps refresher periods after initial use.

The devices and methods of the invention can be used in the treatment and alleviation of various movement pathologies, i.e., tics in head muscle action, inappropriate leg movements in the elderly, excessive falling. Involuntary head movements or tics are a serious accompaniment of Tourette syndrome, a condition characterized by the presence of excessive dopamine in specific brain areas. Dopamine is a neurotransmitter, i.e., a chemical released by neurons to send signals to other neurons or nerve cells. The tics are socially embarrassing, interfere with normal motor behavior, and lead to great psychological stress. The vibration device, by altering feedback to affected musculature, particularly those controlled by the 5th and 7th cranial nerves, have the potential to non-invasively block some forms of tic behavior.

The devices and methods of the invention can be used in the treatment, alleviation and reducing thresholds for epilepsy. A technique useful for reducing the incidence of epilepsy is stimulation of the trigeminal (Vth) or the 10th cranial nerve. Conventional procedures are invasive, requiring a nerve cuff placed over the vagus, and an implanted stimulation device. The stimulus is electrical, with long-term stimulation raising the potential for nerve injury, Stimulation of the cutaneous auricular branch of the vagus would provide a non-invasive means to achieve a similar outcome.

Embodiments of the devices and method described herein can be used to treat potentially harmful breathing problems in babies who were born prematurely. Each year, about 150,000 babies are born after only 23 to 34 weeks of gestation, which puts them at risk for apnea of prematurity, a condition in which breathing stops, often for several seconds, accompanied by severe falls in oxygenation. The condition occurs because infants have systems that are not fully formed, and in-turn the respiratory system ignores or cannot use the body's signals to breathe. Compounding the danger, premature newborns' lungs are not fully developed, and therefore do not have much oxygen in reserve. When breathing stops in these periods of apnea, the level of oxygen in the body goes down, and the heart rate can drop. That combination can damage the lungs and eyes, injure the nerves to the heart, affect the hormonal system (which can lead to diabetes later in life), or injure the brain (which can result in behavioral problems later in life).

Over time, human bodies have developed a system to help the body when running as suddenly more oxygen is needed. In one embodiment, methods of using the devices described herein trick the babies' brain into thinking they are running, which prompts them to breathe. When feet hit the ground running, humans flex muscles and joints that have nerve fibers leading to the brain which signal that the body is running. This message is coupled with another set of fibers to parts of the brain that regulate breathing and sends a signal that those parts need to increase breathing. Fortunately, that coupling exists even in extremely young infants. In certain embodiments, embodiments of the device are placed on the skin over the joints of the feet and hands. Without being bound to any particular theory, it is thought by many that early humans ran on all fours, so nerves in the hands are still involved in signaling the brain that the body is running. Once the battery-powered machine is turned on, the disks gently vibrate, which triggers nerve fibers to alert the brain that the limb is moving which prompts the baby to breathe. Advantageously, long-term use of embodiments of the device could decrease breathing pauses, maintain normal oxygen levels, stabilize the cardiovascular system and help improve neurodevelopmental outcomes in preterm infants. Additionally, this change is brought about with a device that is noninvasive, drug-free and has no side effects.

The devices and methods of the invention can be used in the treatment and alleviation of anxiety, depression, and post-traumatic stress. Stimulation of the trigeminal (Vth) or 10th cranial nerve (vagus) has previously been shown effective in reducing signs of depression. The current intervention involves invasive surgical implantation of nerve cuffs, or electrical stimulation of the auricle. The devices and methods of the invention however would avoid such invasive means or potential injury from electrical stimulation.

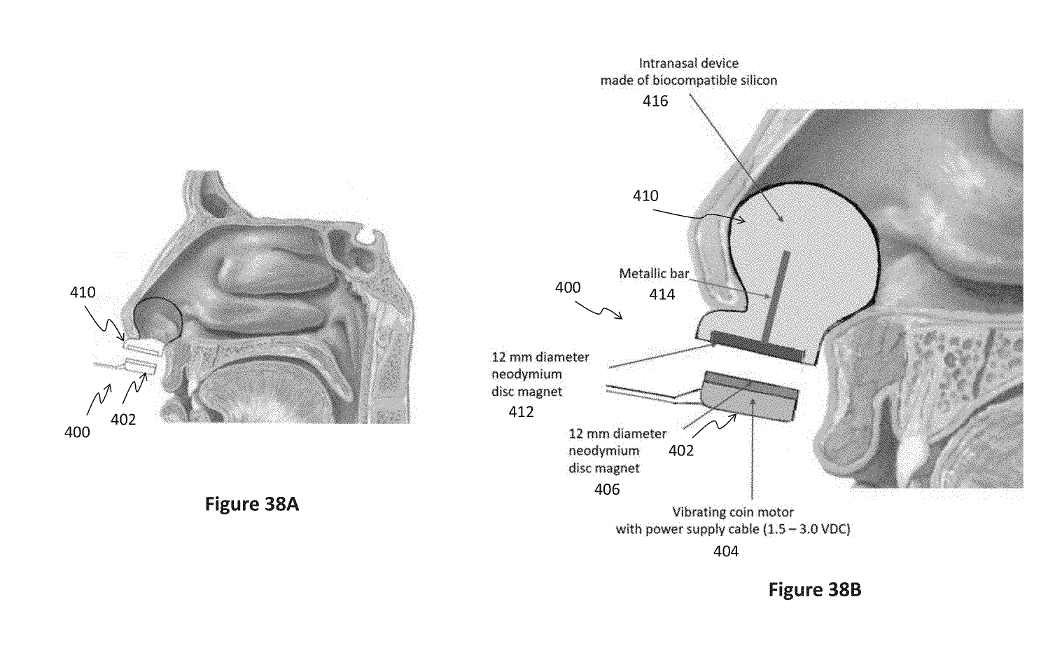

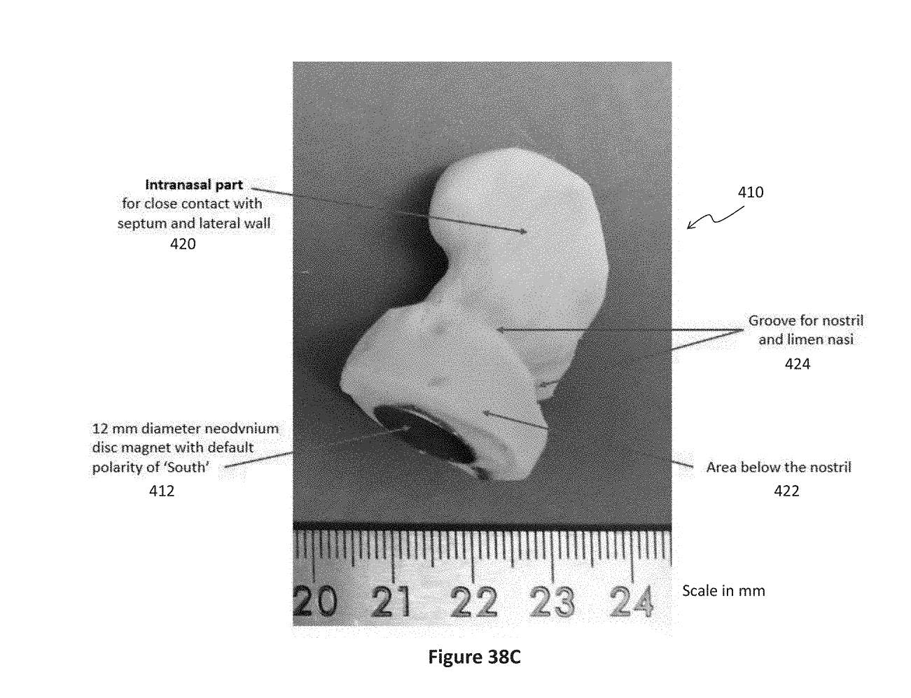

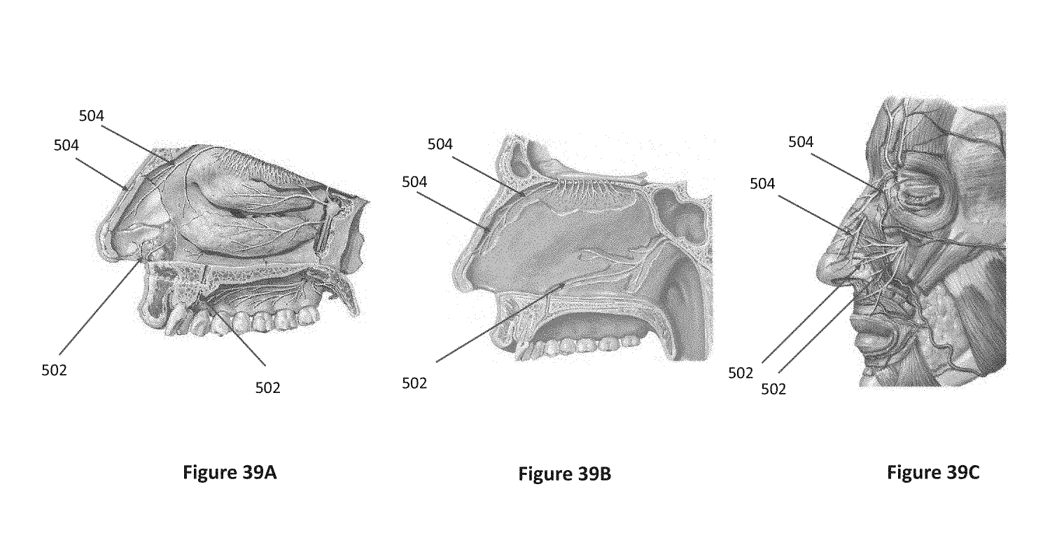

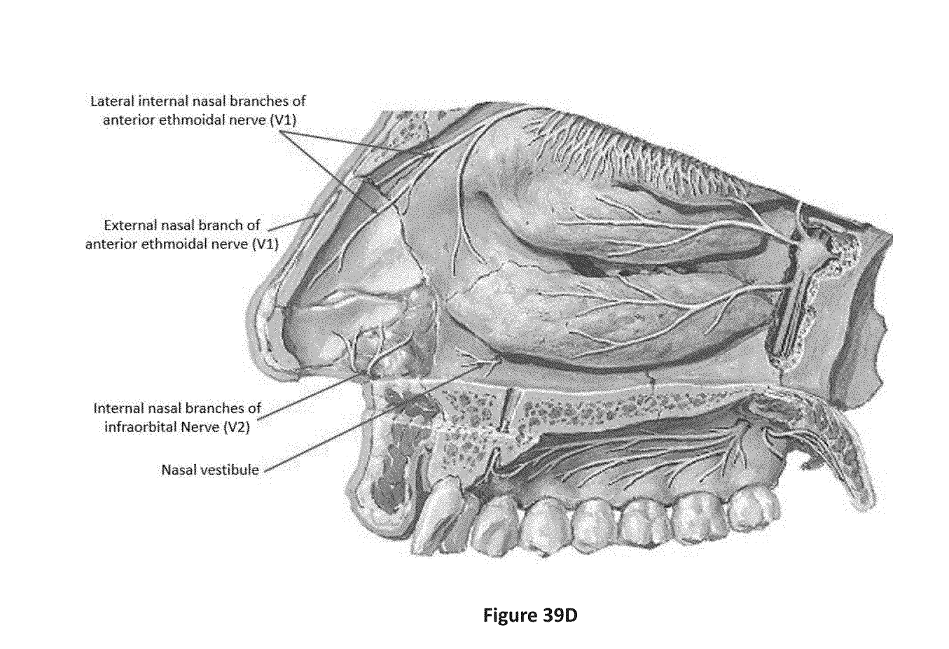

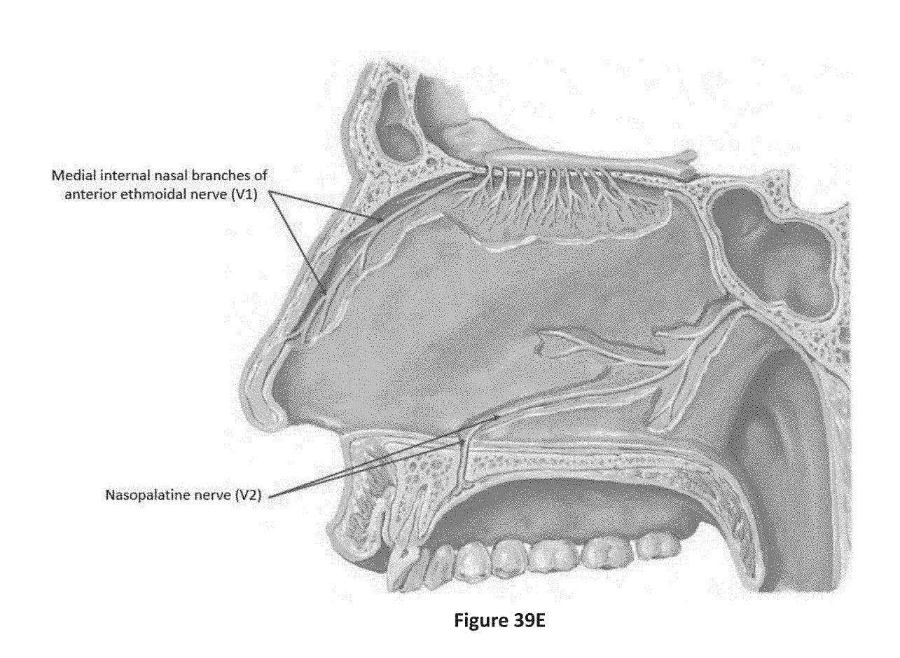

In one embodiment, delivery of the effective stimulus is enhanced by mechanical vibration to the nerves mediating pain from the nasal sinus, dura, retro-orbital region, forehead muscle tension, and oral cavity sites of pain, such as burning mouth syndrome, residual pain from flawed dental procedures, or radiation injury from oral, facial, or nasal oncology procedures. With reference now to FIGS. 38A-38C, in one embodiment, the vibratory device 400 is applied more closely to the source of trigeminal nerves which mediate a substantial proportion of facial, oral, dural, and scalp pain. In one embodiment, a silicon impression 416 of the lower portion of the interior of one nasal cavity is taken, and the magnet 412 is attached to a metallic probe 414 within that impression 416 for vibration transfer. Thus, the component can be entirely passive, composed of non-tissue-reactive silicon material 416 (as used in hearing-aid devices), and an embedded disc magnet 412 attached to an inert metallic bar 414. The silicone material 406 can be formed for close contact with the septum and lateral wall (e.g. FIG. 38C). An intranasal part 420 for close contact with septum and lateral walls can be formed above a part that forms an area below the nostril 422. Grooves 424 can also be formed between these sections 420, 422 in the surface of the silicone for a secure fit with the nostril and limen nasi. The bar can be completely embedded in the silicon material 416. In certain embodiments, there is no electrical contact with the subject, and only vibrations are carried from the vibrating unit 402 to the silicon component 410. In one embodiment, vibration is driven by a vibrating coin motor 404 which latches to the imbedded magnet 412 through a second magnet 406 cemented to the coin motor 404. The device 400 allows vibration to be carried in close proximity to two divisions of the trigeminal nerve, V1 and V2, and thus brings vibration closer to sensory nerves rather than to diffuse sites over the external skin surface. The device provides a non-invasive, non-heating procedure to provide stimulation to the sensory branches of V1 and V2 with mechanical, non-electrical means. In certain embodiments, larger fit devices may have increased effectiveness because they are in contact with a larger nasal surface. Vibrations of the embodiments affect several branches of the second trigeminal division=V2 (e.g. arrows 502), with reference to FIGS. 39A-C. It also affects several branches of the first trigeminal division=V1 (e.g. arrows 504). The device provides stimulation to branches of the trigeminal nerve which contain parasympathetic ganglia, and the autonomic nerves within those autonomic ganglia will also be activated, a major advantage for those with loss of salivation which often accompanies certain trigeminal neuropathies, such as burning mouth syndrome or nerve damage following radiation intervention for parotid or other cancers. In one embodiment, the vibrating device is constructed from a silicon impression for one side of the nose while the other nares is open for breathing, extending within the nares from 10 mm to approximately 25 mm deep. In one embodiment, targeted nerves of the lateral wall of the nasal cavity are shown in FIG. 39D, and targeted nerves of the medial wall (septum) of the nasal cavity are shown in FIG. 39E.

One embodiment for manufacturing the nasal device will now be described. A small square of plastic (e.g., Saran) flexible wrap is inserted into one nasal cavity by the subject using his/her small (5th) finger until a comfortable depth is reached; the wrap is used to prevent direct contact of the silicon paste material with the nasal linings. Because plastic wrap is extruded at temperatures in excess of 150° C., it is sterile as manufactured, and if handled in such a way that there is minimal opportunity for contamination before it is unrolled for use, such contamination is minimized. The semi-fluid silicon material is then filled into the cavity formed by the depression of the sheet of plastic wrap. A 12-mm diameter disc magnet with a small attached holding metallic bar is then inserted into the material, and the silicon left to harden (approximately 4-8 minutes). The impression is then removed, together with the protective wrap. A coin motor with attached magnet and power cables is then used to vibrate the impression when re-inserted during trials. The device can be made in variable lengths, including a short form (e.g. about 10 mm), or a longer version (e.g., about 25 mm). The precise length depends on the unique anatomy of each individual. The purpose of the plastic sheet is to separate the nasal lining from the still-pliable silicon material used for the mold. The plastic sheet is exceptionally impervious to tearing or puncture. The passive elements (the disc magnet with its attached bar) are inserted into the silicon material while it is still-pliable. The device and protective sheet is removed when the silicon sets, and the device (without the sheet) can be reinserted immediately. The surface of the silicon is smooth, non-tissue reactive, and unlikely to irritate the nasal cavity.

The device contribution to pain reduction in trigeminal areas is more marked than regional surface vibrators. The vibration is described as soothing, and leads to remarkable relaxation. The only side effect is that all branches of the trigeminal carry parasympathetic nerve fibers; these fibers are concurrently activated with trigeminal stimulation. That outcome is typically advantageous, since many cases of trigeminal neuropathy are accompanied by dry mouth syndrome, which is remedied with such parasympathetic activation. The impression process with the nasal device is much more comfortable, faster, and simpler than impressions used for the auditory meatus in an earlier vibration intervention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

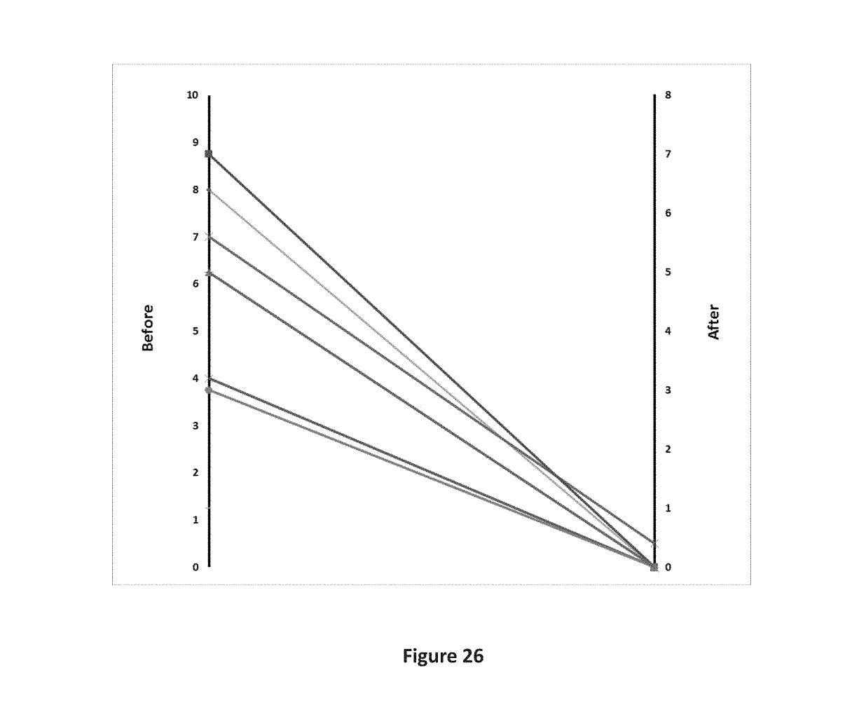

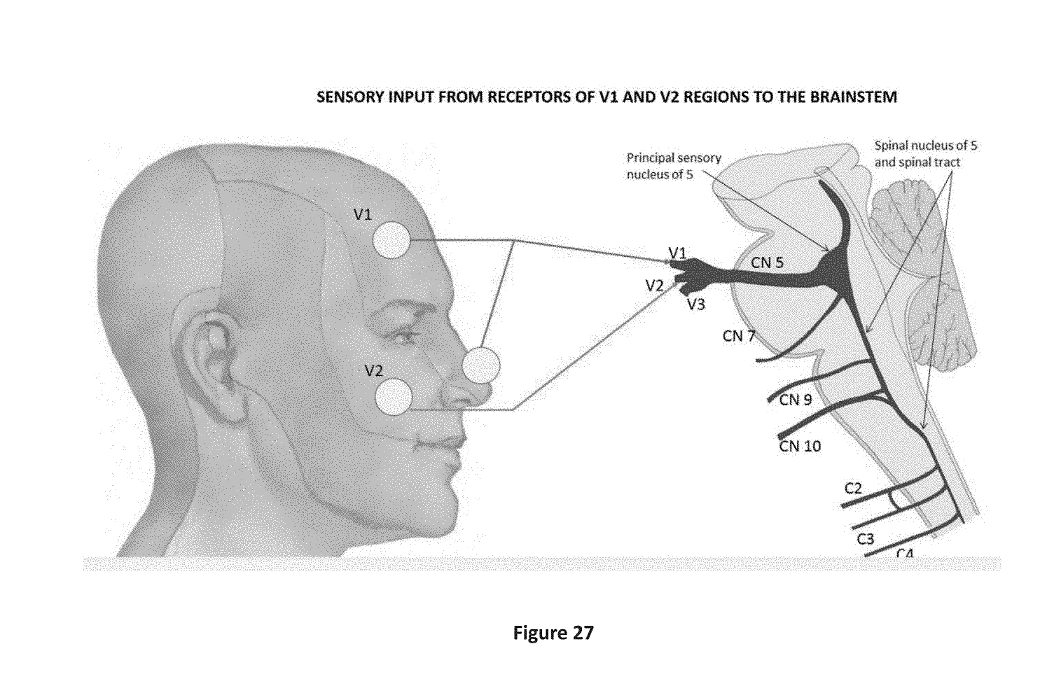

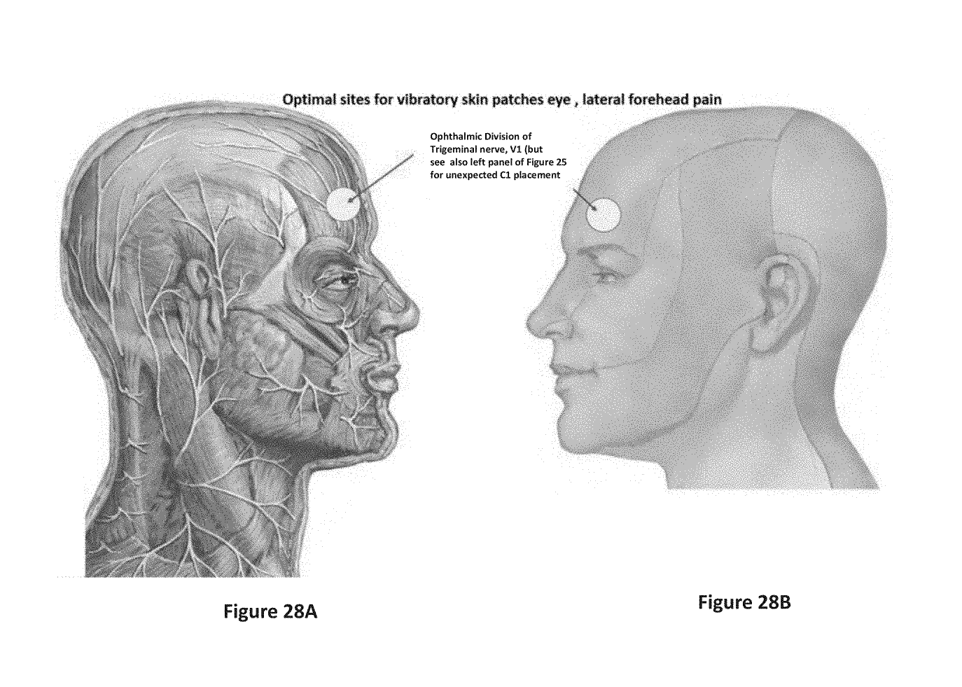

In an experimental example, pain perception was examined in 6 subjects with migraine pain (3 subjects), nasal sinus pain (two patients) and cervical pain (one patient). A vibration motor device of the invention was used to stimulate the respective nerves relevant for attenuating the pain in each case. The subjects were asked to rate their pain on a scale from one to ten before and after a certain period of stimulation, typically 20 minutes. As shown in FIG. 26, which depicts a chart plotting the before and after ratings, the ratings consistently declined, typically from a 4 or 9 level on a 10-point pain scale, to between 0 and 1 within 20 minutes of stimulation.

### Pain Scale

The Numerical Rating Score for Pain (NRS) was completed by the subject at onset of the first trial, when the patient reports that he/she is undergoing moderate-to-severe pain, and at the end of the experimental session. This scale is an uni-dimensional single item scale that provides an easy-to-administer and score scale that allows subjects to rate pain from 0-10 in intensity, and is widely used in the pain field (Hawker et al., Measures of adult pain, Arthritis Care and Research, 2011, 63:S240-S252). It requires about 1-2 minutes to administer.

The following pain scale (1-10) is used to classify pain from the subjects:

1: Very mild=Very light, barely noticeable pain, like a mosquito bite or a poison ivy itch. Most of the time you never think about the pain.

2: Uncomfortable=Minor pain, like lightly pinching the fold of skin between the thumb and first finger with the other hand, using the fingernails.

3: Tolerable=Very noticeable pain, like an accidental cut, a blow to the nose causing a bloody nose, or a doctor giving you a shot. The pain isn't so strong that you can't get used to it.

4: Distressing=Strong, deep pain, like an average toothache, the initial pain from a bee sting, or minor trauma like stubbing your toe real hard. So strong that you notice the pain all the time and can't completely adapt.

5: Very distressing=Strong, deep, piercing pain, such as a sprained ankle when you stand on it wrong, or mild back pain. Not only do you notice the pain all the time, you are now so preoccupied with managing it that your normal lifestyle is curtailed.

6: Intense=Strong, deep, piercing pain, so strong that it seems to partially dominate your senses, causing you to think somewhat unclearly. Comparable to a bad non-migraine headache combined with several bee stings or a bad back pain.

7: Very intense=Same as 6, except that the pain completely dominates your senses causing you to think unclearly about half the time.

8: Utterly horrible=Pain so intense that you can no longer think clearly at all, and have often undergone severe personality change if the pain has been present for a long time. Comparable to childbirth or a real bad migraine.

9: Excruciating unbearable=Pain so intense that you can't tolerate it and demand pain killers or surgery, no matter what the side effects or risk.

10: Unimaginable unspeakable=Pain so intense that you will go unconscious shortly.

### Example 2

A pilot study of the device on reducing blood pressure shows a significant decline of extreme hypertension in two subjects, with pre-stimulation systolic blood pressure levels of 187 and 176 mmHg, reduced by 20-35 mm Hg. The declines are shown in FIG. 37.

### Example 3

In an experimental example, a method of using neuromodulation of proprioceptive fibers to support breathing issues is disclosed, such as issues associated with Apnea of Prematurity (AOP). AOP is common, affecting the majority of infants born at <34 weeks gestational age, with incidence varying inversely with gestational age and birth weight, and appearing in nearly all infants born <29 weeks gestation or <1,000 g [Robertson et al., 2009]. Over half of neonates show AOP at 30 to 31 weeks, 15% at 32 to 33 weeks, and 7% at 34 to 35 weeks gestation [Martin et al., 2004]. The concern with these aberrant breathing patterns is that periodic breathing and apnea are accompanied by intermittent hypoxia (IH), the sequential and repetitive exposure to low oxygen, followed by a rapid increase in oxygen [Poets et al., 1991]. Ventilatory and perfusion disturbances from such breathing patterns are associated with significant cardiovascular sequelae, and contribute to multiple neural pathologies, including neurocognitive and affective disturbances in adults and adolescents [Kumar et al., 2009; Cross et al., 2008]. Sustained or chronic intermittent hypoxia increases free radical production and contributes to the pathogenesis of adverse outcomes associated with obstructive apnea in adults [Sunderram et al., 2012] and children [Bass et al., 2004]. In neonates, the patterns are associated with retinopathy of prematurity, altered growth and cardiovascular regulation, and neurodevelopmental disabilities [Di Fiore et al., 2010; Martin et al., 2011].

The current standard of care for AOP and IH includes prone positioning, continuous positive airway pressure (CPAP) or nasal intermittent positive pressure ventilation (NIPPV) to prevent pharyngeal collapse and alveolar atelectasis, and methylxanthine therapy (caffeine, theophylline) to block adenosine receptors, the mainstay of central apnea treatment [Pantalitschka et al., 2009; Gizzi et al., 2012; Henderson-Smart et al., 2010; Lodha et al., 2015]. These interventions are not always effective, and are not optimal for early development. Positive pressure can induce lung injury in fragile premature subjects, and the required nasal interfaces and their fixing systems may distort bony facial structures in early life [Tibballs et al., 2003; Pape et al., 1976]. Caffeine and theophylline can interfere with sleep [Hayes et al., 2007; Olini et al., 2013], disrupting potential benefits of integrated sleep on brain development and hormone release [Fadda et al., 1997; Schussler et al., 2006] (demonstrated in adults, but as yet, not in neonates).

The objective was to demonstrate the potential to use inherent reflexive coupling between limb movements and breathing to assist ventilation, and make use of a well-demonstrated finding that walking, running, or even passive limb movement will enhance breathing in both animal models and humans [Eldridge et al., 1985; Iscoe et al., 1985; Potts et al., 2005; Fink et al., 1995]. This breathing assistance occurs despite the absence of major changes in the principal drive to ventilation, i.e., carbon dioxide (CO₂) [Forster et al., 2014]. Walking or running are obviously unavailable in premature neonates, but the principle of using sensory information associated with limb movement to reflexively couple with breathing offers a potential to enhance ventilation. Limb movement is simulated in neonates in this study by mild vibratory stimulation of proprioceptors in the hand and foot. A simple, noninvasive vibratory device was placed over proprioceptors of the sole of the foot and the palm of the hands. In certain embodiments, the vibratory device can be devices disclosed herein, such as the embodiments shown in FIGS. 17-23F or others. Mild vibration was designed to activate proprioceptive fiber discharge similar to that arising from limbs during walking or running; since the reflexive coupling with breathing is evolutionarily ancient, the forelimbs, i.e., the hands, can also be used.

Oxygen saturation (SpO₂), breathing, and heart rate patterns in premature neonates (23-34 wks gestation) were compared during periods with and without proprioceptive stimulation. It was hypothesized that activation of proprioceptive fibers using noninvasive vibration would decrease apnea, induced IH episodes, and bradycardia events, and minimize O₂ saturation changes that accompany apnea in premature infants.

Infants who were born between 23 weeks, 0 days of gestation, and 34 weeks and 6 days of gestation were eligible for enrollment after 1 week from birth. Subjects were recruited by referral from the primary care team, as well as self-selection. Only infants demonstrating clinical evidence of AOP with IH episodes at the beginning of the study were enrolled. Caffeine treatment was not a reason for exclusion. Neonates known to have major congenital anomalies/malformations that would influence the CNS and long-term outcomes, e.g., cardiac malformations (except for patent ductus arteriosus or ventricular septal defect), or major neurological malformations, e.g., meningoencephalocele, neonates with apnea from airway issues (e.g., laryngomalacia or severe gastroesophageal reflux disease), and neonates with a history of hypoxic ischemic encephalopathy or Grade IV intraventricular hemorrhage were excluded.

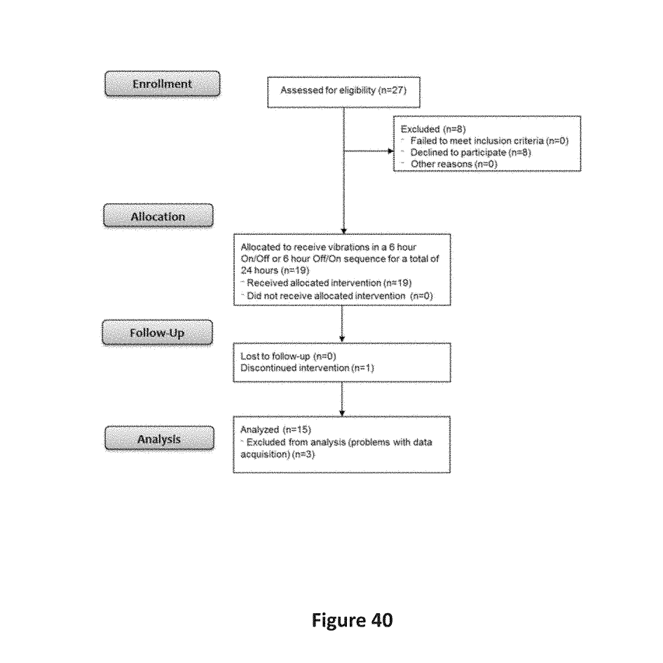

The order for vibration to the infant was randomized by coin flip to begin with or without vibration (FIG. 40). Subjects were monitored for 24 hours with the existing standard NICU monitors (GE Solar 8000i Monitors, GE HealthCare Systems), and proprioceptive stimulation was induced with the vibration devices. The vibration device consisted of two components: a stimulation device, containing a low voltage battery that powers a vibration motor, and small vibrating disks (approximately 10 mm diameter, 3 mm thick). The vibration disks were placed on the palm or wrist of one hand and the ankle or sole of one foot with the hand and the foot on the same side of the infant; sides were randomly selected. The vibration motor is similar to those found in cell phones. The vibration devices delivered continuous mild vibration (0.3 gm/128 Hz) for a 6 hour ON/OFF or OFF/ON sequence, for a total of 24 hours. In all subjects, heart rate via 3 leads, thoracic wall movement for detection of respiratory patterns, and oxygen saturation using pulse oximetry with averaging time of 8 seconds was continuously collected from the existing GE HealthCare monitors that are used in the NICU. In certain embodiments, the vibratory device can be devices disclosed herein, such as the embodiments shown in FIGS. 17-23F or others.

Respiratory, pulse oximetry, and ECG signals were continuously recorded and downloaded to a laptop device with an analog-to-digital converter (NI DAQ 6218 and NI DAQ 6001, National Instruments, Austin Tex.), at 250 samples/second for the 24 hours of the study. Breathing pauses, counted as episodes >3-5 sec in duration (short pauses), and >5 sec in duration (long pauses), the number of IH episodes, determined as the number of events in which 0 saturation fell below 90%, 88%, and 85% for at least 5 sec, and the number of bradycardia episodes were evaluated. A number of AOP studies define significant bradycardia as any decline in heart rate to two-thirds of baseline OR a drop of 30-33% from baseline [Poets et al., 1993; Henderson-Smart et al., 1986; Moriette et al., 2010]. Since the baseline heart rate for our study population was between 150-165 bpm, 100 and 110 bpm was chosen as the threshold for bradycardia.

The total number and duration of breathing pauses, IH episodes and bradycardia episodes were evaluated using LabView Software (S1 Software; National Instruments, Austin, Tex.), as well as LabChart Pro (AD Instruments), with proprioceptive stimulation (total of 12 hours) and without stimulation (total 12 hours), in each study subject. Thus, each subject underwent two 6-hour periods of proprioceptive stimulation and no stimulation, a total of 12 hours of each condition. Stimulation levels were determined based on short stimulation trials to avoid arousal, and durations of stimulation and nonstimulation periods were chosen to obtain an adequate representation of sleep-waking states with different respiratory and cardiovascular patterns. This initial study was designed to study the short-term effectiveness of a vibration device on apnea incidence, desaturation episodes, and cardiovascular measures (HR) during vibration periods, in comparison to baseline (no vibration periods) in the same subject. The bedside nurses and parents were not blinded to the order of vibration/no vibration sequences. However, the respiratory patterns, O₂ saturations, and heart rate data were analyzed by an independent person blinded to the study conditions. A total of 15 infants were analyzed.

Statistical analyses were conducted using IBM SPSS Statistics 23 [IBM 2013]. Within subject differences between periods with stimulation and those without stimulation were analyzed using paired t-tests with an alpha significance level of 0.05. Kolmogorov-Smirnov and Shapiro-Wilk tests of normality were conducted. Deviations from normality were detected, and transformations were performed to adjust for those distributions. Multiple transformations were tested [e.g., squareroot, log 10(x+1)], and ultimately an ln(x+1) transformation was chosen, as it achieved optimal normality [Bland et al., 1996]. All statistical analyses were carried out on the transformed data, and mean and standard error of the raw data were reported for ease of interpretation. Percent change in each variable for each participant is reported in S1 Table.

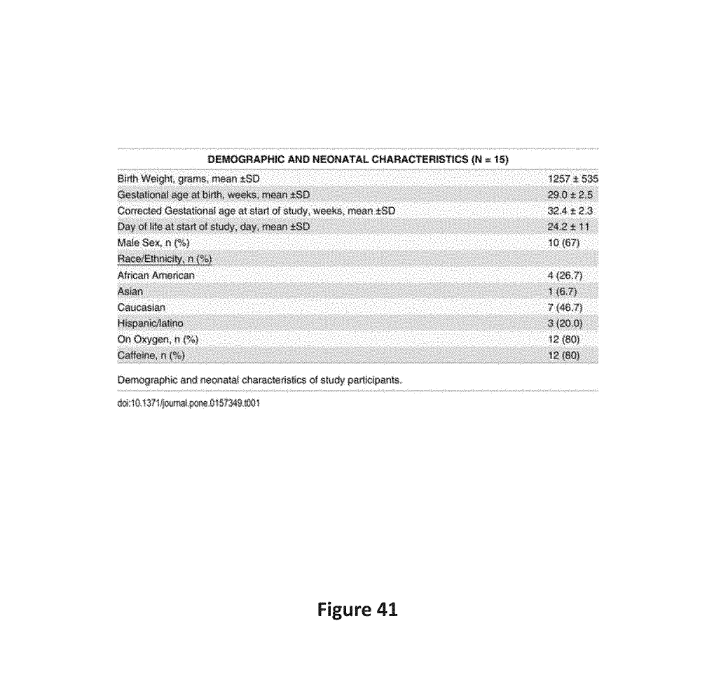

A total of 19 preterm infants (≥23-34 weeks gestational age) were recruited after 1 week of age and randomized to receive vibrations per protocol. In one study subject, the study interventions were discontinued due to worsening of clinical status from sepsis; three additional subjects were excluded from the final analysis due to data acquisition issues (one was missing several hours of data, and two were placed on incompatible monitoring systems following transfer to the lower-level NICU), leaving a final sample size of 15. A third of the study subjects were randomized to the ON/OFF sequence and the rest to OFF/ON. The average gestational age at the study onset was 32±2.3 weeks. The majority of the infants received caffeine for AOP (80%) at the time of the study, and 80% of study subjects were on supplemental oxygen (range 23-50%), via nasal cannula, high flow nasal cannula, or noninvasive ventilation (FIG. 41). None of the infants on the study were endotracheally intubated or received invasive mechanical ventilation while on the study. The primary outcome measure was the change in number of breathing pauses. The secondary outcomes were number of IH episodes and bradycardic episodes.

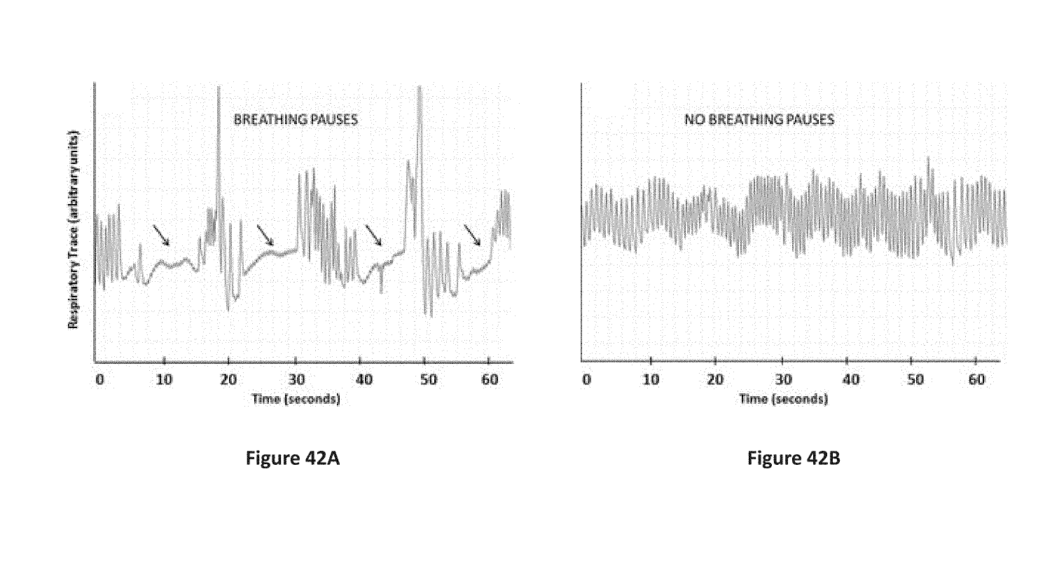

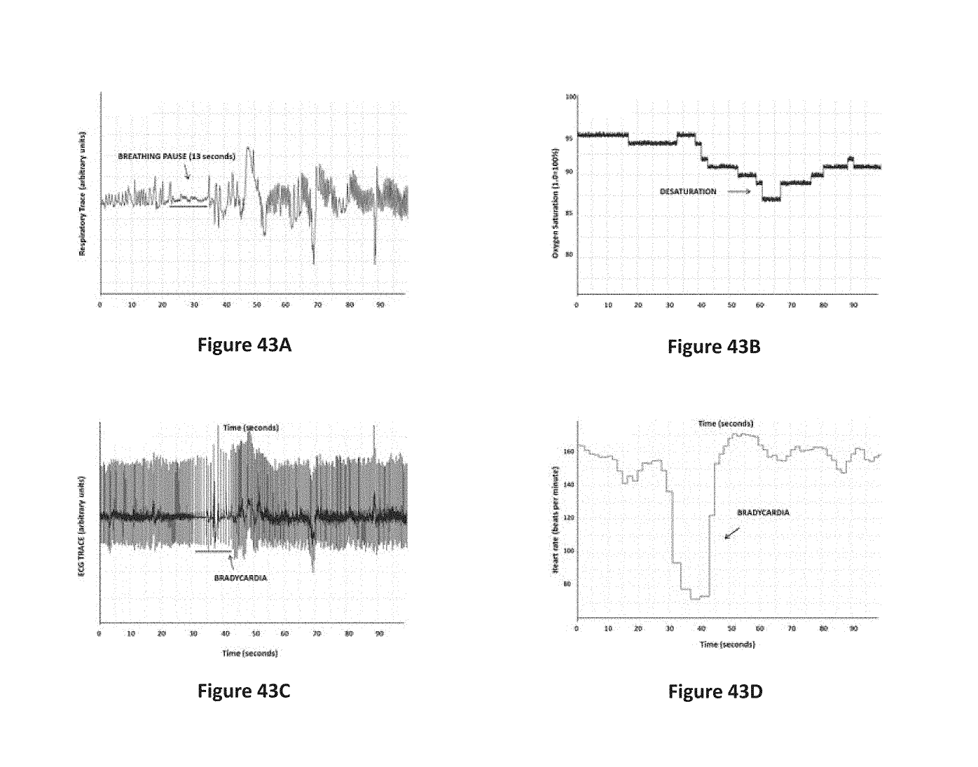

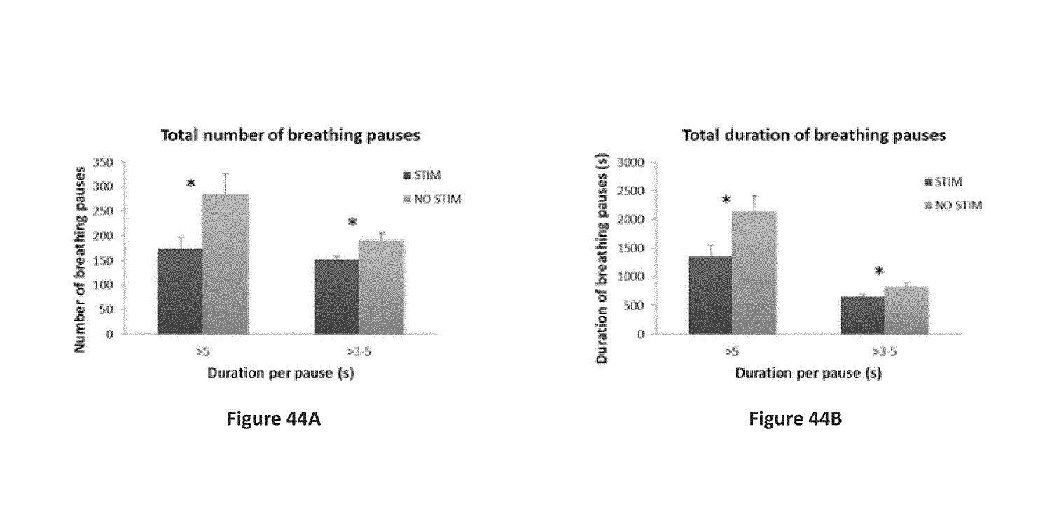

The total number of short and long pauses and total duration of both types of pauses were calculated, and compared in periods with and without stimulation (FIGS. 42A and 42B). Long breathing pauses were frequently accompanied by bradycardia and desaturation (FIGS. 43A-43D). Proprioceptive stimulation significantly reduced the total number of long breathing pauses by 39% (MD=110 pauses, t=7.769, p<0.001), and the number of short breathing pauses by 21% (MD=39 pauses, t=2.536, p=0.024), as compared to periods without proprioceptive stimulation (FIG. 44A). Proprioceptive stimulation significantly reduced the total duration of long breathing pauses by 36% (MD =773 seconds, t=6.681, p<0.001), and stimulation significantly reduced the total duration of short breathing pauses by 20% (MD=166 seconds, t=2.352, p=0.034;

FIG. 44B). Proprioceptive stimulation appeared to significantly lower the number and duration of long breathing pauses in premature neonates with apnea of prematurity.

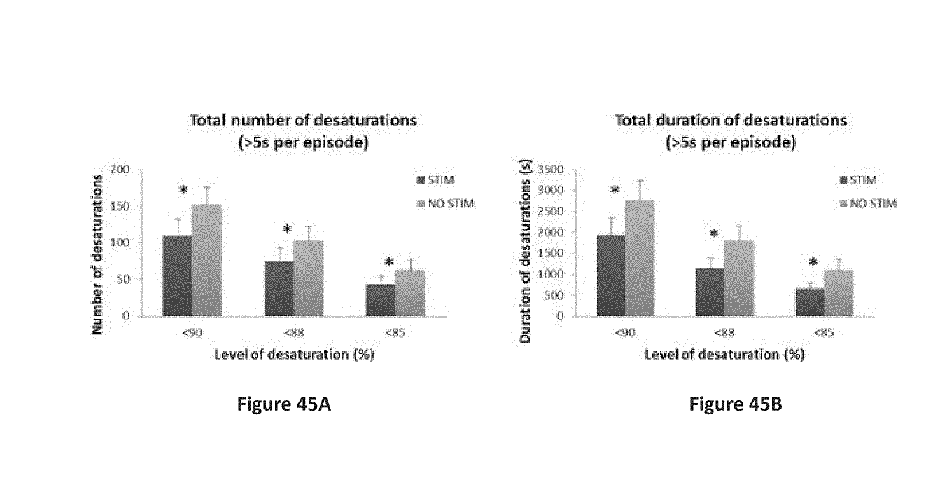

An IH episode was defined as an oxygen desaturation declining to <90%, with duration of at least 5 sec. The total number and duration of IH episodes were compared with and without stimulation. Proprioceptive stimulation significantly reduced the number of IH episodes (MD=42 episodes, t=4.124, p=0.001; FIG. 45A), with a 28% decline in the number of IH episodes with stimulation vs the number without stimulation. The number of IH episodes reaching <88% O saturation was also significantly lower with stimulation vs no stimulation (MD=28 episodes, t=4.022, p=0.001; FIG. 45A). The number of episodes of IH with desaturation declining to <85% also was reduced; that number was significantly lower with stimulation (MD=20 episodes, t=4.633, p<0.001; FIG. 45A). Proprioceptive stimulation significantly reduced the total duration of IH episodes, with a 30% time reduction with stimulation, compared to no stimulation (MD=836 seconds, t=3.689, p=0.002; FIG. 45B). The total durations of desaturations to <88% and <85%, were also significantly lower with stimulation in both categories (MD=655 seconds, t=4.620, p<0.001, and MD=444 seconds, t=2.550, p=0.023, respectively; FIG. 45B). Both the total number and duration of IH episodes of <90%, <88% and <85%, lasting at least 5 sec, were significantly reduced by proprioceptive stimulation in premature neonates.

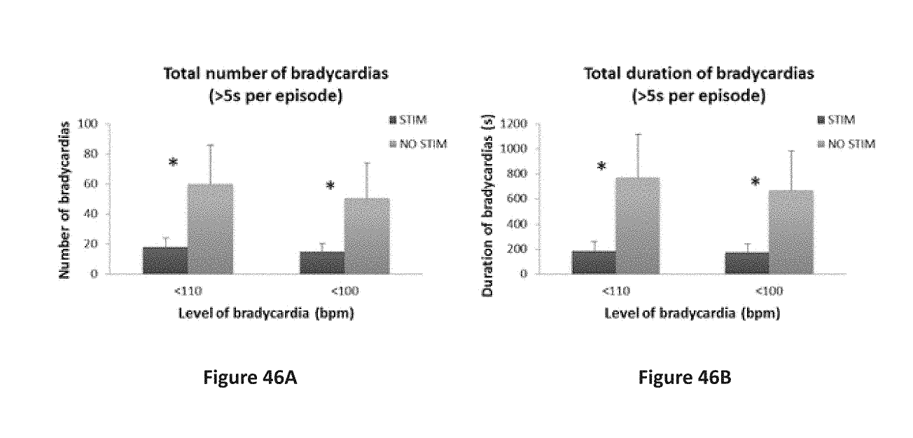

Significantly fewer mild and moderate bradycardia episodes occurred with proprioceptive stimulation. A 3-fold reduction in both mild (<110 bpm) and moderate (<100 bpm) bradycardia episodes emerged with stimulation, compared to no stimulation (MD=42 episodes, t=3.954, p=0.001, and MD=36 episodes, t=3.739, p=0.002, respectively; FIG. 46A). A 3-fold reduction in the total duration of both mild and moderate bradycardia episodes also appeared with stimulation (MD=584 sec, t=3.562, p=0.003, and MD=494 sec, t=3.197, p=0.006, respectively; FIG. 46B). During the total stimulation period of 12 hours, an average total of 185±298 sec of mild bradycardia and 172±255 sec of moderate bradycardia appeared, compared to 769±1346 sec of mild bradycardia and 666±1242 sec of moderate bradycardia in the 12 hrs without stimulation. Both the total number and duration of mild and moderate bradycardia episodes were significantly lower with proprioceptive stimulation.

The findings of this study have both theoretical and pragmatic implications. The intervention, neuromodulation by vibration of afferent proprioceptive fibers to recruit respiratory efferent systems, provides a non-invasive, simple means to reduce apnea of prematurity, the accompanying oxygen desaturation, and the resulting bradycardia, all of which have been implicated in serious developmental consequences for a very common condition in premature neonates. The intervention also demonstrates the close interactions between sensory signals mimicking limb movement and central breathing coordination areas, and shows how precise neuromodulation of appropriate afferent fibers can synchronize breathing patterns essential for vital function. The potential value to neonatal health and subsequent developmental outcomes should not be underestimated. AOP contributes substantially to hospitalization length [Eichenwald et al., 1997; Darnall et al., 1997], and imposes significant, often long-term health concerns. Periods of apnea are accompanied by intermittent hypoxia (IH), hypercapnia, and arousals, with arousals having the potential to disturb sleep state integrity. Both animal and human evidence show that IH exposure contributes to multiple pathophysiologic concerns via proinflammatory and prooxidant cascades, as well as cellular processes, such as apoptosis [Martin et al., 2011; Ryan et al., 2005; Nanduri et al., 2009]. Simulations of apnea modeling IH in animals show damage to sympathetic ganglia regulating cardiovascular action, injury to cerebellar Purkinje cells [Pozo et al., 2012; Lin et al. 2008; Pae et al., 2005], severe hippocampal injury with accompanying memory deficits [Xu et al., 2004], and substantial injury to basal forebrain and neurotransmitter systems [Veasey et al., 2004]. In newborn animals, the damage extends to hampered insulin production, predisposing to diabetes in later life, impaired bone development, lung injury leading to bronchopulmonary dysplasia (BPD) and cerebellar injuries [Pae et al., 2014; Kim et al., 2016; Ratner et al., 2009; Pae et al., 2011]. IH episodes in human neonates lead to acute and chronic morbidities, including retinopathy of prematurity, impaired growth and cardiovascular regulation, bronchopulmonary dysplasia, sleep disordered breathing and neurodevelopmental disabilities [Di Fiorre et al. 2010; Martin et al., 2011; Martin et al., 2015; Cohen et al., 2007; Hibbs et al., 2008; Janiver et al., 2004]. The consequences of successive arousals that disturb sleep states in premature infants are unclear, but are suspected of contributing to multiple pathologies in adult sleep disordered breathing, and especially to hormonal release and glucose regulation [Pae et al., 2014; Grimaldi et al., 2014; Pae et al., 2013]. The need to intervene for AOP is essential for healthy development.

The current approaches to manage AOP and IH focus on a) prevention of pharyngeal collapse and alveolar atelectasis with use of positive pressure ventilation (mechanical ventilation, CPAP, or NIPPV), and b) alleviation of central apneas with pharmacologic agents, such as methylxanthines (caffeine). The lungs of very preterm infants are easily damaged by mechanical ventilation [Pae et al., 2013]. CPAP nasal interfaces and their fixing systems can distort the bony facial structure in early development [Tibballs et al., 2003; Pape et al., 1976]. The objectives of this study did not focus on replacing caffeine with proprioceptive stimulation as a means of reducing apnea. However, it is important to note that caffeine use imposes concerns; its effects on breathing are variable, i.e., it is sometimes ineffective, and concerns linger for later consequences of pharmacologic treatment in a developing infant. Although caffeine therapy decreases the number of apneas [Henderson-Smart et al., 2010], its effect on desaturation is controversial [Bucher et al., 1988; Rhein et al., 2014], and caffeine is not recommended for prophylactic use in premature neonates at risk for AOP [Henderson-Smart et al., 2010]. Caffeine may decrease the rate of BPD and improve survival in very low birth weight infants at 18-21 months, but at 5 years of age its use does not affect rates of survival without disability [Schmidt et al., 2012; Dobson et al., 2014; Patel et al., 2013]. Disparate findings emerge with caffeine effects on inflammation, with both increased proinflammatory cytokines beyond therapeutic doses and inflammatory [Xie et al., 2011] or anti-inflammatory outcomes in newborn rodents [Köroğlu et al., 2014]. Early caffeine use increases the risk of necrotizing enterocolitis [Taha et al., 2014]. Finally, caffeine blocks adenosine, a sleep promoting agent [Brown et al., 2012], thereby enhancing arousals and interfering with the integrity of sleep states [Hayes et al., 2007; Olini et al., 2013]; however, the extent of sleep or other disturbance from caffeine use is controversial [Marcus et al., 2014; Curzi-Dascalova et al., 2002]. Thus, it is apparent that current management strategies for alleviating symptoms of AOP (breathing pauses, IH episodes and bradycardias) may not be adequate.

The finding that limb motion can increase breathing has been noted anecdotally, with observations of synchronized breathing patterns with leg movements, and it has been documented in both animals and humans [Eldridge et al., 1985; Iscoe et al., 1976; Potts et al., 2005; Fink et al., 1995]. Proprioceptive afferents from moving limbs coordinate locomotion and respiratory rhythm generation in humans [Iwamoto et al., 2010]. Frequency of breathing and ventilation immediately increase at the onset of passive limb movements, even during sleep [Ishida et al., 1993]. The usefulness of such limb movement has been demonstrated in congenital central hypoventilation syndrome (CCHS) [Paton et al., 1993; Gozal et al., 1996; Gozal et al., 2000]. CCHS children exhibit sustained cessation of all breathing effort during sleep, rather than the typical periodic breathing characteristic of AOP; however, the common concern in both conditions is hypoventilation. Since sustained mechanical limb flexion and extension is not reasonably feasible in newborn infants, activation of brain areas governing movement that reflexively couple brain areas mediating breathing is needed. For this purpose, fibers carrying kinesthetic cues from the limbs were stimulated to mimic limb tone and motion. This is the first study to use neuromodulation of proprioceptive fibers to support breathing in AOP. Sustained proprioceptive stimulation significantly decreases the number and duration of breathing pauses, IH episodes and bradycardias associated with AOP. The concept of using kinesthetic stimulation for infant breathing support has a long history, with procedures ranging from oscillating waterbeds, vibrating mattresses, and rocking to anecdotal use of foot taps by nursing staff to decrease apneas [Bloch-Salisbury et al., 2009; Korner et al., 1975; Jones et al., 1981; Saigal et al., 1986]. A Cochrane Review in 2002 found no support for prophylactic kinesthetic stimuli via oscillating mattresses, but did not preclude the potential benefit in preterm infants with AOP [Osborn et al., 2000].

A principal advantage of the neuromodulation technique used here, vibratory stimulation of proprioceptive fibers, is the absence of reliance on CO₂ stimulation to drive breathing. The vibration triggers sensory activation that is reflexively relayed to respiratory coordination areas to increase respiratory muscle activation, and the resulting increase in ventilation with motor action is independent of variation in CO₂ drive [Pan et al., 1986]. The independence from CO₂ stimulation is an important aspect in premature infants with AOP, because ventilatory responses to increasing CO₂ are immature, secondary to diminished central sensitivity to CO₂. Moreover, the effector components, the respiratory muscles, including the diaphragm and intercostal muscles, are also immature [Frantz et al., 1976; Keens et al., 1978; Guthrie et al., 1980; Darnall et al., 2010].

A significant concern with any intervention that involves afferent stimulation is the potential to disturb the integrity of sleep states. Breathing and sleep states are closely related, with apneas occurring more often during active sleep; arousal from active sleep is often a precursor to apnea associated with IH episodes in premature neonates [Lehtonen et al., 2004; Malcolm et al., 2009]. A vibrating mattress study found consistently improved respiratory stability using stimuli below thresholds for state changes [Bloch-Salisbury et al., 2009]. In our study, the vibration was mild, with devices applied only to kinesthetic areas for limb motion, with levels intentionally established to minimize arousals. The localized placement of the vibration unit (sole of foot, palm of hand) provided more-focused stimulation than offered by an oscillating mattress or mechanosensory vibrating mattresses. Sleep states were not systematically recorded with electroencephalographic procedures, but onset of vibration did not elicit arousals from sleep, and there were no reports from bedside nursing that sleep states were affected adversely. Premature infants with AOP/IH and exposure to xanthines in early life are at increased risk for sleep-disordered breathing in childhood and adulthood [Hibbs et al., 2008; Paavonen et al., 2007; Rosen et al., 2003]. That finding raises the speculation that the intervention here may improve sleep state integrity, and by removing the injury induced by repeated arousals, may reduce sleep disturbances and sleep disordered breathing in later life.

Apneas that last longer than 15 sec, or are accompanied by bradycardia and desaturations, are considered to be clinically significant. However, even a 5-10 sec breathing pause can be associated with bradycardia or decline in SpO₂. Recurrent IH episodes and bradycardia that follow breathing pauses can elicit neural changes that lead to a higher incidence of death and poor neurodevelopmental outcomes, such as cerebral palsy and blindness at 3 years of age [Janvier et al., 2004; Pillekamp et al., 2007]. Here, it is shown that proprioceptive stimulation decreases the incidence and duration of breathing pauses, IH episodes and bradycardic events, but has the most substantial effect on the number and duration of bradycardias, decreasing the incidence by a factor of 3. Since the presence of bradycardias results from transient large increases in vagal outflow, typically in response to substantial rises in blood pressure, the potential for impaired perfusion of cerebral and other areas is high, with an increased possibility of neural injury. Long-term use of this intervention in premature infants with evidence of apnea, bradycardia and desaturations would be an important next step to determine its effects on neurodevelopmental outcomes.

Neuromodulation of proprioceptive afferents using a vibratory device over areas populated by such afferents provides a low cost, non-invasive means to reduce apnea, O₂ desaturation, and bradycardia in premature infants with AOP. Mechanical vibration of the proprioceptive afferents provides a less injurious and arousing means of stimulation than electrical stimulation. The process makes use of inherent neural reflexive pathways to increase ventilation with limb movement, with movement stimuli replaced with mechanical activation of fibers that normally sense limb motion. The intervention possesses major advantages over conventional positive pressure ventilation techniques, which can damage the young lung and remodel facial structure in premature infants. Moreover, the intervention may decrease the use of pharmacologic agents, which can be ineffective, pose issues with sleep state integrity, and cause unclear changes to developing neural structures. The relief of desaturation and bradycardia episodes has the potential to improve long-term neurodevelopmental and pulmonary outcomes.

### Example 4

In this example, the objective is to support breathing and maintain BP in premature infants by using proprioceptive stimulation via a non-invasive vibratory device, an intervention using the principle that limb movements trigger reflexive facilitation of breathing. Passive motion activates foot region, but also diaphragmatic control motor areas (cervical region) in control adolescents. Passive foot movement recruits respiratory phase switching areas in dorsal midbrain/parabrachial pons & cerebellum. In this example, infants were less than 36 weeks gest. age with AOP. Vibrations were 12 h ON/12 h OFF or 8 h ON/4 h OFF for 3 days. The control group experienced no vibrations. IH episodes, breathing pauses/apneas, bradycardia episodes, BP fluctuations with apnea (PTT) and sleep states using aEEG were compared.

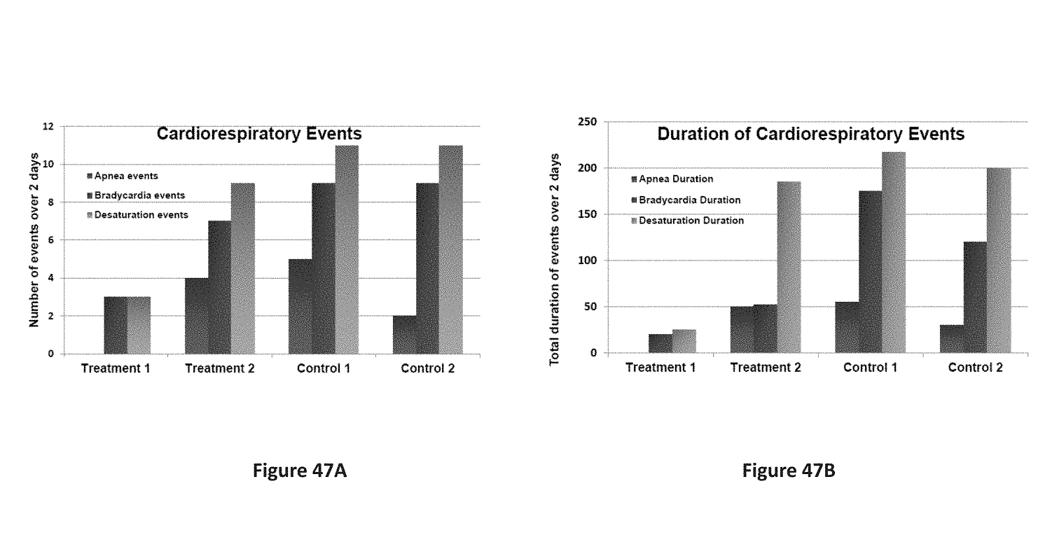

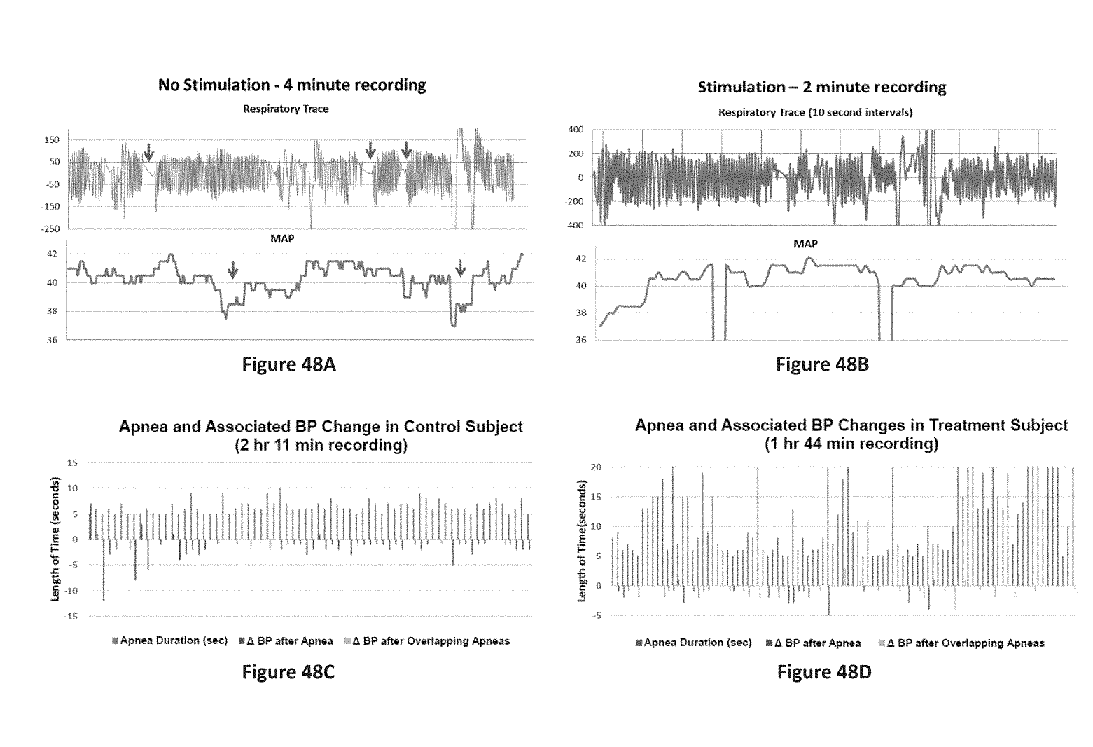

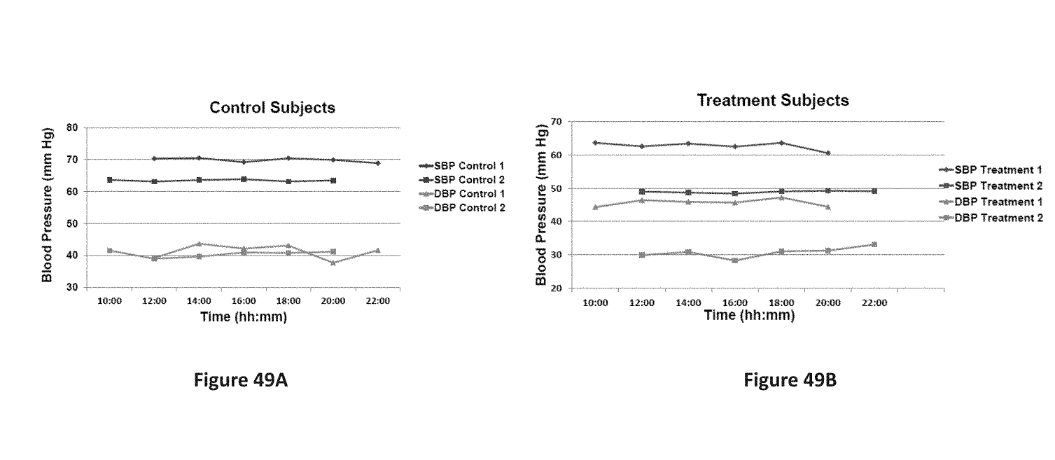

As shown in FIGS. 47A-B, Proprioceptive stimulation reduces bradycardia and desaturation events and duration. Treatment subjects 1 & 2 had 0 & 4 apnea events whereas control subjects 1 & 2 had 5 & 2 apnea events respectively. Treatment subjects had fewer bradycardia events compared to control subjects (3 & 7 vs. 9 & 9 respectively) and also had fewer desaturation events (3 & 9 vs. 11 & 11 respectively). The duration of bradycardias and desaturation events were longer in control subjects (bradycardias 175 & 120 sec and desaturation 217 & 200 sec) compared to treatment subjects (bradycardias 20 & 52 sec and desaturation 25 & 185 sec). [Apnea-breathing pause ≥5 sec, bradycardia event—heartrate <100 beats per min for ≥5 sec, desaturation event—oxygen desaturation to ≤90% for ≥5 sec.] As shown in FIGS. 48A-D, proprioceptive stimulation maintains BP and decreases fluctuations. Respiratory pauses (arrows) during non-stimulation periods were accompanied by acute falls in MAP (arrows) soon after breathing pause. These traces show continuous beat-by-beat BP measures, derived from PTT calculated by the SOMNOmedics acquisition device, and calibrated with a conventional cuff BP (Rapid vertical drops-artifact). Control subject had more severe BP fluctuations, up to even 10 mmHg, not noted in the treatment subject, who appears to have more apneas, but less severe BP fluctuations. Diurnal BP trends in control and treatment subjects are shown in FIGS. 49A-B. Both SBP and DBP remain stable during the entire day without exhibiting diurnal variation in both control and treatment subjects.

In summary, fewer desaturation events occurred in treatment subjects vs control subjects; fewer bradycardic events occurred in treatment subjects compared to controls; and acute changes in BP accompanying apneic events were reduced in treatment subjects receiving vibration compared to control subjects not receiving vibration.

### REFERENCES

Akerman et al., Pearls and pitfalls in experimental in vivo models of migraine: Dural trigeminovascular nociception. Cephalagia, 2013, 33 (8) 577-592
Bass J L, Corwin M, Gozal D, Moore C, Nishida H, Parker S, et al. The effect of chronic or intermittent hypoxia on cognition in childhood: a review of the evidence. Pediatrics. 2004; 114(3):805-16
Bland J M, Altman D G. Statistics notes: transforming data. BMJ. 1996; 312(7033):770
Bloch-Salisbury E, Indic P, Bednarek F, Paydarfar D. Stabilizing immature breathing patterns of preterm infants using stochastic mechano-sensory stimulation. Journal of applied physiology (Bethesda, Md.: 1985). 2009; 107(4):1017-27
Brown R E, Basheer R, McKenna J T, Strecker R E, McCarley R W. Control of sleep and wakefulness. Physiol Rev. 2012; 92(3):1087-187
Bucher H U, Duc G. Does caffeine prevent hypoxaemic episodes in premature infants? A randomized controlled trial. European journal of pediatrics. 1988; 147(3):288-91
Clancy et al., Non-invasive vagus nerve stimulation in healthy humans reduces sympathetic nerve activity. Brain Stimul., 2014 November-December; 7(6):871-7. doi: 10.1016/j.brs.2014.07.031. Epub 2014 Jul. 16. PMID: 25164906
Cohen G, Lagercrantz H, KatzSalamon M. Abnormal circulatory stress responses of preterm graduates. Pediatric research. 2007; 61(3):329-34
Cross R L, Kumar R, Macey P M, Doering L V, Alger J R, YanGo F L, et al. Neural alterations and depressive symptoms in obstructive sleep apnea patients. Sleep. 2008; 31(8):1103-9
Cukiert et al., Vagus Nerve Stimulation for Epilepsy: An Evidence-Based Approach, Prog Neurol Surg. 2015 Sep., 29:39-52. doi: 10.1159/000434654. Epub 2015 Sep. 4. PMID: 26393531
Curzi-Dascalova L, Aujard Y, Gaultier C, Rajguru M. Sleep organization is unaffected by caffeine in premature infants. The Journal of pediatrics. 2002; 140(6):766-71
Darnall R A, Kattwinkel J, Nattie C, Robinson M. Margin of safety for discharge after apnea in preterm infants. Pediatrics. 1997; 100(5):795-801
Darnall R A. The role of CO(2) and central chemoreception in the control of breathing in the fetus and the neonate. Respiratory physiology & neurobiology. 2010; 173(3):201-12
DaSilva et al., tDCS-Induced Analgesia and Electrical Fields in Pain-Related Neural Networks in Chronic Migraine. Headache, The Journal of Head and Face Pain, 2012; 52: 1283-1295
Davis et al., Reversal of central sleep apnea following discontinuation of opioids, J Clin Sleep Med. 2012 Oct., 15; 8(5):579-80, doi: 10.5664/jcsm.2164. PMID: 23066372
Di Fiore J M, Bloom J N, Orge F, Schutt A, Schluchter M, Cheruvu V K, et al. A higher incidence of intermittent hypoxemic episodes is associated with severe retinopathy of prematurity. The Journal of pediatrics. 2010; 157(1):69-73.
Dobson N R, Patel R M, Smith P B, Kuehn D R, Clark J, Vyas-Read S, et al. Trends in caffeine use and association between clinical outcomes and timing of therapy in very low birth weight infants. The Journal of pediatrics. 2014; 164(5):992-8.e3
Eichenwald E C, Aina A, Stark A R. Apnea frequently persists beyond term gestation in infants delivered at 24 to 28 weeks. Pediatrics. 1997; 100(3 Pt 1):354-9
Eldridge F L, Millhorn D E, Kiley J P, Waldrop T G. Stimulation by central command of locomotion, respiration and circulation during exercise. Respiration physiology. 1985; 59(3):313-37
Fadda P, Fratta W. Stress-induced sleep deprivation modifies corticotropin releasing factor (CRF) levels and CRF binding in rat brain and pituitary. Pharmacological research. 1997; 35(5):443-6
Fink G R, Adams L, Watson J D, Innes J A, Wuyam B, Kobayashi I, et al. Hyperpnoea during and immediately after exercise in man: evidence of motor cortical involvement. The Journal of physiology. 1995; 489 (Pt 3):663-75
Forster H V. Recent advances in understanding mechanisms regulating breathing during exercise. The Journal of physiology. 2014; 592(3):429-31.
Frangos et al., Non-invasive access to the vagus nerve central projections via electrical stimulation of the external ear: fMRI evidence in humans. Brain Stimul., 2015 May-June; 8(3):624-36. doi: 10.1016/j.brs.2014.11.018. Epub 2014 Dec. 6. PMID: 25573069
Frantz I D 3rd, Adler S M, Thach B T, Taeusch H W Jr. Maturational effects on respiratory responses to carbon dioxide in premature infants. Journal of applied physiology. 1976; 41(1):41-5
Gizzi C, Papoff P, Giordano I, Massenzi L, Barbara C S, Campelli M, et al. Flow-synchronized nasal intermittent positive pressure ventilation for infants <32 weeks' gestation with respiratory distress syndrome. Critical care research and practice. 2012; 2012:301818
Gozal D, Marcus C L, Ward S L, Keens T G. Ventilatory responses to passive leg motion in children with congenital central hypoventilation syndrome. American journal of respiratory and critical care medicine. 1996; 153(2):761-8
Gozal D, Simakajornboon N. Passive motion of the extremities modifies alveolar ventilation during sleep in patients with congenital central hypoventilation syndrome. American journal of respiratory and critical care medicine. 2000; 162(5):1747-51.
Grimaldi D, Beccuti G, Touma C, Van Cauter E, Mokhlesi B. Association of obstructive sleep apnea in rapid eye movement sleep with reduced glycemic control in type 2 diabetes: therapeutic implications. Diabetes care. 2014; 37(2):355-63
Guthrie R D, Standaert T A, Hodson W A, Woodrum D E. Sleep and maturation of eucapnic ventilation and CO₂ sensitivity in the premature primate. Journal of applied physiology: respiratory, environmental and exercise physiology. 1980; 48(2):347-54
Hartwell et al., Sleep disturbances and pain among individuals with prescription opioid dependence, Addict Behav, 2014 Oct.; 39(10):1537-42, doi: 10.1016/j.addbeh.2014.05.025. Epub 2014 Jun. 2. PMID: 24999989
Hayes M J, Akilesh M R, Fukumizu M, Gilles A A, Sallinen B A, Troese M, et al. Apneic preterms and methylxanthines: arousal deficits, sleep fragmentation and suppressed spontaneous movements. J Perinatol. 2007; 27(12):782-9
Henderson et al., Somatotopic organization of the processing of muscle and cutaneous pain in the left and right insula cortex: A single-trial fMRT study. Pain, 2007, 128, 20-30
Henderson et al., Chronic pain: lost inhibition? J Neurosci., 2013 Apr. 24; 33(17):7574-82
Henderson-Smart D J, ButcherPuech M C, Edwards D A. Incidence and mechanism of bradycardia during apnoea in preterm infants. Arch Dis Child. 1986; 61(3):227-32
Henderson-Smart D J, De Paoli A G. Methylxanthine treatment for apnoea in preterm infants. The Cochrane database of systematic reviews. 2010; (12):CD000140
Henderson-Smart D J, De Paoli A G. Prophylactic methylxanthine for prevention of apnoea in preterm infants. The Cochrane database of systematic reviews. 2010; (12):CD000432
Hibbs A M, Johnson N L, Rosen C L, Kirchner H L, Martin R, Storferlsser A, et al. Prenatal and neonatal risk factors for sleep disordered breathing in school-aged children born preterm. The Journal of pediatrics. 2008; 153(2):176-82
Hoffmann et al., Inhibition of histamine-induced bronchoconstriction in guinea pig and swine by pulsed electrical vagus nerve stimulation, Neuromodulation Technol Neural Interface, 2009 Oct. 1; 12(4):261-9
IBM. IBM SPSS Statistics for Windows, Version 23.0. IBM Corp Armonk, N.Y.; 2013
Iscoe S, Polosa C. Synchronization of respiratory frequency by somatic afferent stimulation. Journal of applied physiology (Bethesda, Md.: 1985). 1976; 40(2): 138-48
Ishida K, Yasuda Y, Miyamura M. Cardiorespiratory response at the onset of passive leg movements during sleep in humans. European journal of applied physiology and occupational physiology. 1993; 66(6):507-13
Iwamoto E, Taito S, Kawae T, Sekikawa K, Takahashi M, Inamizu T. The neural influence on the occurrence of locomotor-respiratory coordination. Respiratory physiology & neurobiology. 2010; 173(1):23-8
Janetta, P., Neurovascular compression in cranial nerve and systemic disease, Ann Surg, 1980 192 (4):518-525. PMCID: PMC1246998
Janvier A, Khairy M, Kokkotis A, Cormier C, Messmer D, Barrington K J. Apnea is associated with neurodevelopmental impairment in very low birth weight infants. J Perinatol. 2004; 24(12):763-8
Jensen and Brugger, Interpretation of visual analog scale ratings and change scores: A reanalysis of two clinical trials of postoperative pain, The Journal of Pain, 2003, 4:407-414
Jones R A. A controlled trial of a regularly cycled oscillating waterbed and a non-oscillating waterbed in the prevention of apnoea in the preterm infant. Arch Dis Child. 1981; 56(11):889-91
Kim G, Elnabawi O, Shin D, Pae E K. Transient intermittent hypoxia exposure disrupts neonatal bone strength. Front Pediatr. 2016; 4
Keens T G, Bryan A C, Levison H, Ianuzzo C D. Developmental pattern of muscle fiber types in human ventilatory muscles. Journal of applied physiology: respiratory, environmental and exercise physiology. 1978; 44(6):909-13
Korner A F, Kraemer H C, Haffner M E, Cosper L M. Effects of waterbed flotation on premature infants: A pilot study. Pediatrics. 1975; 56(3):361-7
Köroğlu Ö A, MacFarlane P M, Balan K V, Zenebe W J, Jafri A, Martin R J, et al. Anti-inflammatory effect of caffeine is associated with improved lung function after lipopolysaccharide-induced amnionitis. Neonatology. 2014; 106(3):235-40
Lay and Frostig, Complete protection from impending stroke following permanent middle cerebral artery occlusion in awake, behaving rats, European J. Neurosci., 2014. 40, 3413-3421. PMID: 25216240
Lehtonen L, Martin R J. Ontogeny of sleep and awake states in relation to breathing in preterm infants. Seminars in neonatology: S N. 2004; 9(3):229-38
Lin M, Ai J, Li L, Huang C, Chapleau M W, Liu R, et al. Structural remodeling of nucleus ambiguus projections to cardiac ganglia following chronic intermittent hypoxia in C57B L/6J mice. The Journal of comparative neurology. 2008; 509(1):103-17
Lodha A, Seshia M, McMillan D D, Barrington K, Yang J, Lee S K, et al. ASsociation of early caffeine administration and neonatal outcomes in very preterm neonates. JAMA Pediatr. 2015; 169(1):33-8
Malcolm W F, Smith P B, Mears S, Goldberg R N, Cotten C M. Transpyloric tube feeding in very low birthweight infants with suspected gastroesophageal reflux: impact on apnea and bradycardia. J Perinatol. 2009; 29(5):372-5
Marcus C L, Meltzer L J, Roberts R S, Traylor J, Dix J, D′ilario J, et al. Long-term effects of caffeine therapy for apnea of prematurity on sleep at school age. American journal of respiratory and critical care medicine. 2014; 190(7):791-9
Martin R J, AbuShaweesh J M, Baird T M. Apnoea of prematurity. Paediatric respiratory reviews. 2004; 5 Suppl A:S377-82
Martin R J, Wang K, Koroglu O, Di Fiore J, Kc P. Intermittent hypoxic episodes in preterm infants: do they matter? Neonatology. 2011; 100(3):303-10
Martin R J, Di Fiore J M, Walsh M C. Hypoxic Episodes in Bronchopulmonary Dysplasia. Clinics in perinatology. 2015; 42(4):825-38
Melzack and Wall, Pain mechanisms: a new theory, Science. 1965 Nov. 19; 150(3699):971-9. PMID: 5320816
Meng et al., Migraine prevention with a supraorbital transcutaneous stimulator: A randomized controlled trial, Neurology, 2013, 81, 1102-1103.PMID: 24042576
Meng et al., Vagus nerve stimulation for pediatric and adult patients with pharmaco-resistant epilepsy, Chin. Med J. 2015, 128 (19) 2599-2604. PMID: 26415797
Miner et al., Feasibility of percutaneous vagus nerve stimulation for the treatment of acute asthma exacerbations, Acad Emerg Med. 2012 Apr. 1; 19(4):421-9
Moriette G, Lescure S, El Ayoubi M, Lopez E. [Apnea of prematurity: what's new?]. Arch Pediatr. 2010; 17(2):186-90
Mosqueira et al., Vagus nerve stimulation in patients with migraine, Rev Neurol, 2013 Jul. 16; 57(2):57-63
Nahas et al., Two-year outcome of vagus nerve stimulation (VNS) for treatment of major depressive episodes, J Clin Psychiatry. 2005 September; 66(9):1097-104. PMID: 16187765
Nanduri J, Wang N, Yuan G, Khan S A, Souvannakitti D, Peng Y J, et al. Intermittent hypoxia degrades HIF2alpha via calpains resulting in oxidative stress: implications for recurrent apnea-induced morbidities. Proceedings of the National Academy of Sciences of the United States of America. 2009; 106(4):1199-204
Olesen and Ashina, Emerging migraine treatments and drug targets, Trends in Pharmacological Sciences 2011, Vol. 32, No. 6, 352-359
Olini N, Kurth S, Huber R. The effects of caffeine on sleep and maturational markers in the rat. PloS one. 2013; 8(9):e72539
Osborn D A, Henderson-Smart D J. Kinesthetic stimulation versus theophylline for apnea in preterm infants. The Cochrane database of systematic reviews. 2000; (2):CD000502
Paavonen E J, Strang-Karlsson S, Raikkonen K, Heinonen K, Pesonen A K, Hovi P, et al. Very low birth weight increases risk for sleep-disordered breathing in young adulthood: the Helsinki study of very low birth weight adults. Pediatrics. 2007; 120(4):778-84
Pae E K, Chien P, Harper R M. Intermittent hypoxia damages cerebellar cortex and deep nuclei. Neuroscience letters. 2005; 375(2):123-8
Pae E K, Yoon A J, Ahuja B, Lau G W, Nguyen D D, Kim Y, et al. Perinatal intermittent hypoxia alters gamma-aminobutyric acid: a receptor levels in rat cerebellum. International Journal of Developmental Neuroscience. 2011; 29(8):819-26
Pae E K, Ahuja B, Kim M, Kim G. Impaired glucose homeostasis after a transient intermittent hypoxic exposure in neonatal rats. Biochem Biophys Res Commun. 2013; 441(3):637-42
Pae E K, Kim G. Insulin production hampered by intermittent hypoxia via impaired zinc homeostasis. PloS one. 2014; 9(2):e90192
Pan L G, Forster H V, Bisgard G E, Murphy C L, Lowry T F. Independence of exercise hyperpnea and acidosis during high-intensity exercise in ponies. Journal of applied physiology (Bethesda, Md.: 1985). 1986; 60(3):1016-24
Pantalitschka T, Sievers J, Urschitz M S, Herberts T, Reher C, Poets C F. Randomised crossover trial of four nasal respiratory support systems for apnoea of prematurity in very low birthweight infants. Archives of disease in childhood Fetal and neonatal edition. 2009; 94(4):F245-8
Pape K E, Armstrong D L, Fitzhardinge P M. Central nervous system pathology associated with mask ventilation in the very low birthweight infant: a new etiology for intracerebellar hemorrhages. Pediatrics. 1976; 58(4):473-83
Patel R M, Leong T, Carlton D P, Vyas-Read S. Early caffeine therapy and clinical outcomes in extremely preterm infants. J Perinatol. 2013; 33(2):134-40
Paton J Y, Swaminathan S, Sargent C W, Hawksworth A, Keens T G. Ventilatory response to exercise in children with congenital central hypoventilation syndrome. The American review of respiratory disease. 1993; 147(5):1185-91
Pedersen et al., Neurostimulation in cluster headache: A review of current progress, Cephalagia, 2013. 33(14) 1179-1193
Petkovich et al., Vagal modulation of hypertension, Curr Hypertens Rep. 2015 Apr.; 17(4):532. doi: 10.1007/s11906-015-0532-6
Pillekamp F, Hermann C, Keller T, von Gontard A, Kribs A, Roth B. Factors influencing apnea and bradycardia of prematurity—implications for neurodevelopment. Neonatology. 2007; 91(3):155-61
Plachta et al., BaroLoop: using a multichannel cuff electrode and selective stimulation to reduce blood pressure, Conf Proc IEEE Eng Med Biol Soc. 2013; 2013:755-8. doi: 10.1109/EMBC.2013.6609610, PMID: 24109797
Poets C F, Southall D P. Patterns of oxygenation during periodic breathing in preterm infants. Early human development. 1991; 26(1):1-12
Poets C F, Stebbens V A, Samuels M P, Southall D P. The relationship between bradycardia, apnea, and hypoxemia in preterm infants. Pediatric research. 1993; 34(2):144-7
Potts J T, Rybak I A, Paton JFR. Respiratory rhythm entrainment by somatic afferent stimulation. The Journal of Neuroscience: the Official Journal of the Society for Neuroscience. 2005; 25(8):1965-78
Pozo M E, Cave A, Koroglu O A, Litvin D G, Martin R J, Di Fiore J, et al. Effect of postnatal intermittent hypoxia on growth and cardiovascular regulation of rat pups. Neonatology. 2012; 102(2):107-13
Ratner V, Slinko S, UtkinaSosunova I, Starkov A, Polin R A, Ten V S. Hypoxic stress exacerbates hyperoxia-induced lung injury in a neonatal mouse model of bronchopulmonary dysplasia. Neonatology. 2009; 95(4):299-305
Rhein L M, Dobson N R, Darnall R A, Corwin M J, Heeren T C, Poets C F, et al. Effects of caffeine on intermittent hypoxia in infants born prematurely: a randomized clinical trial. JAMA Pediatr. 2014; 168(3):250-7
Robertson C M, Watt M J, Dinu I A. Outcomes for the extremely premature infant: what is new? And where are we going? Pediatric neurology. 2009; 40(3):189-96
Rosen C L, Larkin E K, Kirchner H L, Emancipator J L, Bivins S F, Surovec S A, et al. Prevalence and risk factors for sleep-disordered breathing in 8-to-11-year-old children: association with race and prematurity. The Journal of pediatrics. 2003; 142(4): 383-9
Ryan S, Taylor C T, McNicholas W T. Selective activation of inflammatory pathways by intermittent hypoxia in obstructive sleep apnea syndrome. Circulation. 2005; 112(17):2660-7
Saigal S, Watts J, Campbell D. Randomized clinical trial of an oscillating air mattress in preterm infants: effect on apnea, growth, and development. The Journal of pediatrics. 1986; 109(5):857-64
Stavrakis S, Humphrey M B, Scherlag B J, Hu Y, Jackman W M, Nakagawa H, Lockwood D, Lazzara R, Po S S. Low-level transcutaneous electrical vagus nerve stimulation suppresses atrial fibrillation. J Am Coll Cardiol. 2015 Mar. 10; 65(9):867-75. doi: 10.1016/j.jacc.2014.12.026. PMID: 25744003
Schmidt B, Anderson P J, Doyle L W, Dewey D, Grunau R E, Asztalos E V, et al. Survival without disability to age 5 years after neonatal caffeine therapy for apnea of prematurity. Jama. 2012; 307(3):275-82
Schmolzer G M, Te Pas A B, Davis P G, Morley C J. Reducing lung injury during neonatal resuscitation of preterm infants. The Journal of pediatrics. 2008; 153(6):741-5
Schoenen et al., Migraine prevention with a supraorbital transcutaneous stimulator, Neurology, 2013, 0(8):697-704. doi: 10.1212/WNL.0b013e3182825055. Epub 2013 Feb. 6. PMID: 23390177
Schussler P, Uhr M, Ising M, Weikel J C, Schmid D A, Held K, et al. Nocturnal ghrelin, ACTH, G H and cortisol secretion after sleep deprivation in humans. Psychoneuroendocrinology. 2006; 31(8):915-23. Epub 2006/07/04
Silberstein et al., Botulinum Toxin Type A as a Migraine Preventive Treatment, Headache: The Journal of Head and Face Pain. 40, 6, 445-450, 2000
Stavrakis et al., Low-level transcutaneous electrical vagus nerve stimulation suppresses atrial fibrillation, J Am Coll Cardiol. 2015 Mar. 10; 65(9):867-75. doi: 10.1016/j.jacc.2014.12.026.PMID: 25744003
Sunderram J, Androulakis I P. Molecular mechanisms of chronic intermittent hypoxia and hypertension. Critical reviews in biomedical engineering. 2012; 40(4):265-78
Taha D, Kirkby S, Nawab U, Dysart K C, Genen L, Greenspan J S, et al. Early caffeine therapy for prevention of bronchopulmonary dysplasia in preterm infants. J Matern Fetal Neona. 2014; 27(16):1698-702
Tibballs J, Henning R D. Noninvasive ventilatory strategies in the management of a newborn infant and three children with congenital central hypoventilation syndrome. Pediatr Pulmonol. 2003; 36(6):544-8
Veasey S C, Davis C W, Fenik P, Zhan G, Hsu Y J, Pratico D, et al. Long-term intermittent hypoxia in mice: protracted hyper-somnolence with oxidative injury to sleep-wake brain regions. Sleep. 2004; 27(2):194-201
Xie H G, Cao Y J, Gauda E B, Agthe A G, Hendrix C W, Lee H. Clonidine clearance matures rapidly during the early postnatal period: a population pharmacokinetic analysis in newborns with neonatal abstinence syndrome. The Journal of Clinical Pharmacology. 2011; 51(4):502-11
Xu W, Chi L, Row B W, Xu R, Ke Y, Xu B, et al. Increased oxidative stress is associated with chronic intermittent hypoxia-mediated brain cortical neuronal cell apoptosis in a mouse model of sleep apnea. Neuroscience. 2004; 126(2):313-23
Yu et al., Low-level transcutaneous electrical stimulation of the auricular branch of the vagus nerve: a noninvasive approach to treat the initial phase of atrial fibrillation, Heart Rhythm. 2013 March; 10(3):428-35. doi: 10.1016/j.hrthm.2012.11.019. Epub 2012 Nov. 24. PMID: 23183191
Hu et al., Burden of migraine in the United States: disability and economic costs, Arch. Intern. Med., 1999, 159, 813-818
White et al., Neuromodulation of cranial nerves for migraine and trigeminal neuropathy pain: cardiac effects. FASEB E B, San Diego, B91 731.4, 2016
Deep brain stimulation for cluster headaches: http://www.bbc.co.uk/news/health-12013583

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations

What is claimed is:

1. A system for stimulating one or more sensory fibers of a nerve, comprising:
a device comprising a vibration source and a first magnet in contact with the vibration source, such that vibrational energy is capable of transferring to the first magnet; and
a second magnet;
wherein the first magnet and the second magnet are releasably engageable via a magnetic field.

2. The system of claim 1, wherein the second magnet is further attached to an adhesive patch.

3. The system of claim 1, wherein the vibration source is a vibration motor.

4. The system of claim 1, wherein the vibration source is connected to and powered by a control unit.

5. The system of claim 4, wherein the control unit is controlled by a computing device.

6. The system of claim 5, wherein the computing device controls the control unit wirelessly.

7. A system for stimulating one or more sensory fibers of a nerve, comprising:
a device comprising a vibration source and a first mechanical connector in contact with the vibration source, such that vibrational energy is capable of transferring to the mechanical connector; and
a second mechanical connector attached to an adhesive patch;
wherein the first mechanical connector and the second mechanical connector are releasably engageable via friction or other type of mechanical retention.

8. The system of claim 7, wherein the vibration source is a vibration motor.

9. The system of claim 7, wherein the first mechanical connector is a snap connector.

10. The system of claim 9, wherein the snap connector is a female snap connector.

11. The system of claim 7, wherein the vibration source is connected to and powered by a control unit.

12. The system of claim 11, wherein the control unit is controlled by a computing device.

13. The system of claim 12, wherein the computing device controls the control unit wirelessly.

14. A method of stimulating one or more sensory or motor fibers of a nerve in a subject,
wherein the nerve is selected from the group consisting of ophthalmic nerve V1, maxillary nerve V2, mandibular nerve V3, cranial nerve 5, cranial nerve 7, cranial nerve 9, cranial nerve 10, spinal nerve C1, spinal nerve C2, spinal nerve C3, and spinal nerve C4, and sensory and motor nerves of remaining spinal nerves from C4 to sacral 5.
the method comprising placing a vibration source device on or proximal to the skin of the subject.

15. The method of claim 14, wherein the vibration source device further comprises a first magnet, the method comprising placing the first magnet of the device on a portion of the skin of the subject and providing a second magnet, wherein the first magnet and the second magnet are releasably attracted to each other via a magnetic field through at least a portion of the skin of the subject.

16. The method of claim 14, wherein the vibration source device further comprises a first magnet, the method comprising providing an adhesive patch fitted with a second magnet, and placing the adhesive patch on to the skin of the subject, wherein the two magnets are releasably attached to each other via a magnetic field.

17. The method of claim 14, wherein the vibration source device further comprises a first mechanical connector, the method comprising providing an adhesive patch fitted with a second mechanical connector, placing the adhesive patch on to the skin of the subject, wherein the two mechanical connectors are releasably attached to each other by friction or other type of mechanical retention.

18. The method of claim 14, wherein the stimulation leads to treating, alleviating, or preventing a condition in a subject.

19. The method of claim 18, wherein the condition is selected from the group consisting of pain of the head, pain of the oral cavity, pain of the neck, pain of the shoulder and limbs, pain of the lower back and pudendal area, pain of the nasal sinus, pain of the face, pain of the dura covering the brain.

20. The method of claim 18, wherein the condition is a cardiovascular condition selected form the group consisting of atrial fibrillation, sinus tachycardia, ventricular tachycardia, long Q-T, hypertension, hypotension, or extremely variable blood pressure.

21. The method of claim 18, wherein the condition is selected from the group consisting of an ischemic stroke, a mood disorder, depression, anxiety, post-traumatic stress disorder, epilepsy, movement disorder, including falling, gait disorders, tremor, coughing, swallowing, hiccup, or involuntary tic.

22. The method of claim 18, wherein the condition is selected from the group consisting of obstructive pulmonary disease (COPD), asthma, obstructive sleep apnea, central apnea, Cheyne-Stokes breathing, periodic breathing, and hypoventilation.

23. The method of claim 14, wherein the stimulation leads to retraining breathing in a subject.

24. The method of claim 14, wherein the stimulation leads to induction of sleep in a subject.
